(19) United States
(12) Patent Application Publication
(10) Pub. No.: US 2011/0034441 A1
(43) Pub. Date: Feb. 10, 2011

(54) INDAZOLES AS WNT/B-CATENIN SIGNALING PATHWAY INHIBITORS AND THERAPEUTIC USES THEREOF

(75) Inventors: John Hood, San Diego, CA (US); Sunil Kumar KC, San Diego, CA (US)

Correspondence Address: FISH & RICHARDSON P.C. (TC), PO BOX 1022, MINNEAPOLIS, MN, 55440-1022, US

(73) Assignee: EPITHERIX, LLC, San Diego, CA (US)

(21) Appl. No.: 12/852,681

(22) Filed: Aug. 9, 2010

Related U.S. Application Data
(60) Provisional application No. 61/232,603, filed on Aug. 10, 2009.

(51) Int. Cl. A61K 31/551; A61K 31/4184; A61K 31/454; A61K 31/496; A61K 31/5377; A61K 31/541; A61K 31/437; A61K 31/416; A61K 31/4178; A61K 31/4439; A61P 35/00; A61P 31/10; A61P 31/12; A61P 19/02; A61P 17/06; A61P 27/06; A61P 25/28; A61P 3/10; A61P 3/04; A61P 19/10; A61P 43/00

(52) U.S. Cl. 514/218; 514/394; 514/322; 514/254.06; 514/234.5; 514/228.2; 514/303; 514/403; 514/392; 514/338

(57) ABSTRACT

Described herein are methods of treating a disorder or disease in which aberrant Wnt signaling is implicated, with a variety of compounds, including Wnt inhibitor compounds. More particularly, it concerns the use of an indazole compound or analogs thereof, in the treatment of disorders characterized by the activation of Wnt pathway signaling (e.g., cancer, abnormal cellular proliferation, angiogenesis, Alzheimer's disease and osteoarthritis), the modulation of cellular events mediated by Wnt pathway signaling, as well as genetic diseases due to mutations in Wnt signaling components.

### RELATED APPLICATIONS

### Cross-Reference to Related Applications

This application claims the benefit of U.S. Provisional Application No. 61/232,603, filed Aug. 10, 2009, which is incorporated herein by reference in its entirety.

### BACKGROUND OF THE INVENTION

1. Field of the Invention

This invention relates to inhibitors of one or more proteins in the Wnt pathway, including inhibitors of one or more Wnt proteins, and compositions comprising the same. More particularly, it concerns the use of an indazole compound or salts or analogs thereof, in the treatment of disorders characterized by the activation of Wnt pathway signaling (e.g., cancer, abnormal cellular proliferation, angiogenesis, Alzheimer's disease and osteoarthritis), the modulation of cellular events mediated by Wnt pathway signaling, as well as genetic diseases due to mutations in Wnt signaling components.

2. Description of the Related Art

Pattern formation is the activity by which embryonic cells form ordered spatial arrangements of differentiated tissues. Speculation on the mechanisms underlying these patterning effects usually centers on the secretion of a signaling molecule that elicits an appropriate response from the tissues being patterned. More recent work aimed at the identification of such signaling molecules implicates secreted proteins encoded by individual members of a small number of gene families.

A longstanding idea in cancer biology is that cancers arise and grow due to the formation of cancer stem cells, which may constitute only a minority of the cells within a tumor but are nevertheless critical for its propagation. Stem cells are appealing as the cell of origin for cancer because of their pre-existing capacity for self-renewal and for unlimited replication. In addition, stem cells are relatively long-lived in comparison to other cells within tissues, providing a greater opportunity to accumulate the multiple additional mutations that may be required to increase the rate of cell proliferation and produce clinically significant cancers. Of particular recent interest in the origin of cancer is the observation that the Wnt signaling pathway, which has been implicated in stem cell self-renewal in normal tissues, upon continuous activation has also been associated with the initiation and growth of many types of cancer. This pathway thus provides a potential link between the normal self-renewal of stem cells and the aberrantly regulated proliferation of cancer stem cells.

The Wnt growth factor family includes more than 10 genes identified in the mouse and at least 7 genes identified in the human. Members of the Wnt family of signaling molecules mediate many important short- and long-range patterning processes during invertebrate and vertebrate development. The Wnt signaling pathway is known for its important role in the inductive interactions that regulate growth and differentiation, and likely also plays important roles in the homeostatic maintenance of post-embryonic tissue integrity. Wnt stabilizes cytoplasmic p-catenin, which stimulates the expression of genes including c-myc, c jun, fra-1, and cyclin D1. In addition, misregulation of Wnt signaling can cause developmental defects and is implicated in the genesis of several human cancers. More recently, the Wnt pathway has been implicated in the maintenance of stem or progenitor cells in a growing list of adult tissues that now includes skin, blood, gut, prostate, muscle and the nervous system.

Pathological activation of the Wnt pathway is also believed to be the initial event leading to colorectal cancer in over 85% of all sporadic cases in the Western world. Activation of the Wnt pathway has also been extensively reported for hepatocellular carcinoma, breast cancer, ovarian cancer, pancreatic cancer, melanomas, mesotheliomas, lymphomas and leukemias. In addition to cancer, inhibitors of the Wnt pathway can be used for stem cell research or for the treatment of any diseases characterized by aberrant Wnt activation such as diabetic retinopathy, neovascular glaucoma, rheumatoid arthritis, psoriasis as well as mycotic and viral infections and bone and cartilage diseases. As such, it is a therapeutic target that is of great interest to the field.

In addition to cancer, there are many cases of genetic diseases due to mutations in Wnt signaling components. Examples of some of the many diseases are Alzheimer's disease [Proc. Natl. Acad. Sci. USA (2007), 104(22), 9434-9], osteoarthritis, polyposis coli [Science (1991), 253(5020), 665-669], bone density and vascular defects in the eye (osteoporosis-pseudoglioma syndrome, OPPG) [N. Engl. J. Med. (2002), 346(20), 1513-21], familial exudative vitreoretinopathy [Hum. Mutat. (2005), 26(2), 104-12], retinal angiogenesis [Nat. Genet. (2002), 32(2), 326-30], early coronary disease [Science (2007), 315(5816), 1278-82], tetra-amelia syndrome [Am. J. Hum. Genet. (2004), 74(3), 558-63], Müllerian-duct regression and virilization [Engl. J. Med. (2004), 351(8), 792-8], SERKAL syndrome [Am. J. Hum. Genet. (2008), 82(1), 39-47], diabetes mellitus type 2 [Am. J. Hum. Genet. (2004), 75(5), 832-43; N. Engl. J. Med. (2006), 355(3), 241-50], Fuhrmann syndrome [Am. J. Hum. Genet. (2006), 79(2), 402-8], Al-Awadi/Raas-Rothschild/Schinzel phocomelia syndrome [Am. J. Hum. Genet. (2006), 79(2), 402-8], odonto-onycho-dermal dysplasia [Am. J. Hum. Genet. (2007), 81(4), 821-8], obesity [Diabetologia (2006), 49(4), 678-84], split-hand/foot malformation [Hum. Mol. Genet. (2008), 17(17), 2644-53], caudal duplication syndrome [Am. J. Hum. Genet. (2006), 79(1), 155-62], tooth agenesis [Am. J. Hum. Genet. (2004), 74(5), 1043-50], Wilms tumor [Science (2007), 315(5812), 642-5], skeletal dysplasia [Nat. Genet. (2009), 41(1), 95-100], focal dermal hypoplasia [Nat. Genet. (2007), 39(7), 836-8], autosomal recessive anonychia [Nat. Genet. (2006), 38(11), 1245-7], neural tube defects [N. Engl. J. Med. (2007), 356(14), 1432-7], alpha-thalassemia (ATRX) syndrome [The Journal of Neuroscience (2008), 28(47), 12570-12580], fragile X syndrome [PLoS Genetics (2010), 6(4), e1000898], ICF syndrome, Angelman syndrome [Brain Research Bulletin (2002), 57(1), 109-119], Prader-Willi syndrome [Journal of Neuroscience (2006), 26(20), 5383-5392], Beckwith-Wiedemann Syndrome [Pediatric and Developmental Pathology (2003), 6(4), 299-306] and Rett syndrome.

### SUMMARY OF THE INVENTION

The present invention makes available methods and reagents, involving contacting a cell with an agent, such as an aromatic compound, in a sufficient amount to antagonize a Wnt activity, e.g., to reverse or control an aberrant growth state or correct an genetic disorder due to mutations in Wnt signaling components.

Some embodiments disclosed herein include Wnt inhibitors containing an indazole core. Other embodiments disclosed herein include pharmaceutical compositions and methods of treatment using these compounds.

One embodiment disclosed herein includes a compound having the structure of formula I:

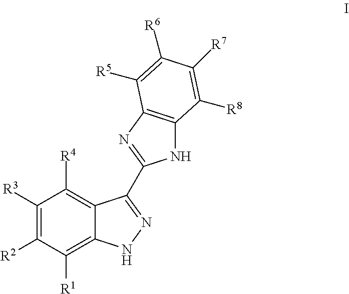

In some embodiments of formula (I):

R¹, R², R⁴, R⁵, R⁶, R⁷ and R⁸ are independently selected from the group consisting of H, C₁₋₉ alkyl, halide, —CF₃, —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)S(═O)R¹⁰, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹;

R³ is selected from the group consisting of —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)S(═O)R¹⁰, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹;

alternatively, one of each R¹ and R², R² and R³, R³ and R⁴, R⁵ and R⁶, R⁶ and R⁷ or R⁷ and R⁸ are taken together to form a ring which is selected from the group consisting of aryl, heteroaryl,

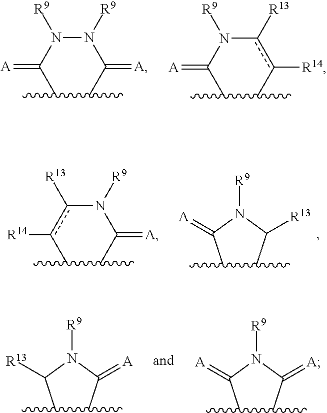

wherein each bond represented by a dashed and solid line represents a bond selected from the group consisting of a single bond and a double bond;

each R⁹ is independently selected from the group consisting of H, —C₁₋₉ alkyl, —CF₃, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;

each R¹⁰ is independently selected from the group consisting of —C₁₋₉ alkyl, —CF₃, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;

each R¹¹ is independently selected from the group consisting of CN, —OR⁹ and R⁹;

each R¹² is 1-5 substituents each selected from the group consisting of H, C₁₋₉ alkyl, -alkylaminoalkyl, halide, —CF₃, carbocyclylR¹², heterocyclylR¹², arylR¹², heteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙNR⁹C(═O)OR⁹, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹;

R¹³ and R¹⁴ are independently selected from the group consisting of H, C₁₋₉ alkyl, halide, —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙN(R⁹)C(=A)R⁹, —NO₂, —CN, —(C₁₋₉ alkyl)ₙCO₂R⁹ and —(C₁₋₉ alkyl)ₙC(=A)R⁹;

alternatively, R¹³ and R¹⁴ are taken together to form a ring which is selected from the group consisting of benzene and pyridine;

each A is independently selected from O, S and NR¹¹; and

each n is 0 or 1, or a pharmaceutically acceptable salt or pro-drug thereof.

Another embodiment disclosed herein includes a compound having the structure of formula II:

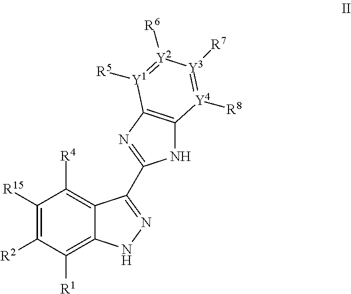

In some embodiments of formula (II):

R¹, R², R⁴, R⁵, R⁶, R⁷ and R⁸ are independently selected from the group consisting of H, C₁₋₉ alkyl, halide, —CF₃, —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)S(═O)R¹⁰, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹;

alternatively, one of each R¹ and R², R² and R¹⁵, R¹⁵ and R⁴, R⁵ and R⁶, R⁶ and R⁷ or R⁷ and R⁸ are taken together to form a ring which is selected from the group consisting of aryl, heteroaryl,

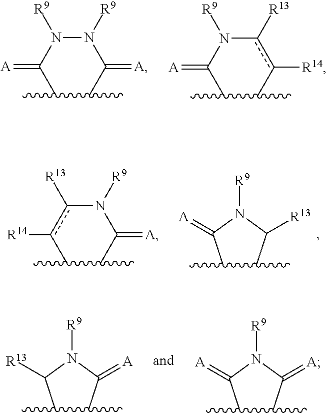

wherein each bond represented by a dashed and solid line represents a bond selected from the group consisting of a single bond and a double bond;

each R⁹ is independently selected from the group consisting of H, —C₁₋₉ alkyl, —CF₃, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;

each R¹⁰ is independently selected from the group consisting of —C₁₋₉ alkyl, —CF₃, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;

each R¹¹ is independently selected from the group consisting of CN, —OR⁹ and R⁹;

each R¹² is 1-5 substituents each selected from the group consisting of H, C₁₋₉ alkyl, -alkylaminoalkyl, halide, —CF₃, carbocyclylR¹², heterocyclylR¹², arylR¹², heteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙNR⁹C(═O)OR⁹, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹;

R¹³ and R¹⁴ are independently selected from the group consisting of H, C₁₋₉ alkyl, halide, —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙN(R⁹)C(=A)R⁹, —NO₂, —CN, —(C₁₋₉ alkyl)ₙCO₂R⁹ and —(C₁₋₉ alkyl)ₙC(=A)R⁹;

alternatively, R¹³ and R¹⁴ are taken together to form a ring which is selected from the group consisting of benzene and pyridine;

each R¹⁵ is selected from the group consisting of -carbocyclylR¹⁶, —(C₁₋₉ alkyl)carbocyclylR¹², -heterocyclylR¹⁶, —(C₁₋₉ alkyl)heterocyclylR¹², -arylR¹⁶, —(C₁₋₉ alkyl)arylR¹², -heteroarylR¹⁶, —(C₁₋₉ alkyl)heteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)N(R⁹)S(═O)R¹⁰, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, —NO₂, —CN, —(C₁₋₉ alkyl)ₙCO₂R⁹ and —(C₁₋₉ alkyl)ₙC(=A)R⁹;

each R¹⁶ is 1-5 substituents each selected from the group consisting of H, C₁₋₉ alkyl, halide, —CF₃, carbocyclylR¹², heterocyclylR¹², arylR¹², heteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)N(R⁹)SO₂R⁹, —(C₁₋₉ alkyl)ₙSO₂N(R⁹)₂, —N(R¹⁷)₂, —(C₁₋₉ alkyl)N(R⁹)₂, —NR⁹C(═O)N(R¹⁷)₂, —(C₁₋₉ alkyl)N(R⁹)C(=A)N(R⁹)₂, —C(═O)NR⁹R¹⁸, —(C₁₋₉ alkyl)C(=A)N(R⁹)₂, —(C₁₋₉ alkyl)NR⁹C(═O)OR⁹, —(C₁₋₉ alkyl)N(R⁹)C(=A)R⁹, —(C₁₋₉ alkyl)OC(═O)N(R⁹)₂, —NO₂, —CN, —(C₁₋₉ alkyl)ₙCO₂R⁹ and —(C₁₋₉ alkyl)C(=A)R⁹;

each R¹⁷ is H, —(C₁₋₉ alkyl)ₙcarbocyclyl, —(C₁₋₉ alkyl)ₙheterocyclyl, —(C₁₋₉ alkyl)ₙaryl and —(C₁₋₉ alkyl)ₙheteroaryl;

each R¹⁸ is independently selected from the group consisting of H, —C₁₋₉ alkyl, —CF₃, -carbocyclylR¹², —(C₁₋₉ alkyl)ₙarylR¹² and —(C₁₋₉ alkyl)ₙheteroarylR¹²;

each A is independently selected from O, S and NR¹¹;

Y¹, Y², Y³ and Y⁴ are independently selected from the group consisting of carbon and nitrogen with the proviso that at least one of Y¹, Y², Y³ and Y⁴ are nitrogen;

If Y¹ is nitrogen then R⁵ is absent;

If Y² is nitrogen then R⁶ is absent;

If Y³ is nitrogen then R⁷ is absent;

If Y⁴ is nitrogen then R⁸ is absent; and

each n is 0 or 1, or a pharmaceutically acceptable salt or pro-drug thereof.

Another embodiment disclosed herein includes a compound having the structure of formula III:

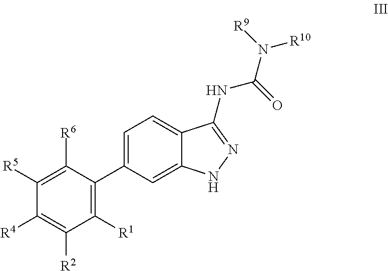

In some embodiments of formula (III):

R¹, R², R⁴, R⁵ and R⁶ are independently selected from the group consisting of H, C₁₋₉ alkyl, halide, —CF₃, —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)S(═O)R¹⁰, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹;

each R⁹ is independently selected from the group consisting of H, —C₁₋₉ alkyl, —(C₁₋₉ alkyl)OR⁹, —(C₁₋₉ alkyl)N(R⁹)₂, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;

each R¹⁰ is independently selected from the group consisting of —C₁₋₉ alkyl, —(C₁₋₉ alkyl)OR⁹, —(C₁₋₉ alkyl)N(R⁹)₂, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;

alternatively, R⁹ and R¹⁰ are taken together to form a 3-10 membered heterocyclyl ring;

each R¹¹ is independently selected from the group consisting of CN, —OR⁹ and R⁹;

each R¹² is 1-5 substituents each selected from the group consisting of H, C₁₋₉alkyl, -alkylaminoalkyl, halide, —CF₃, carbocyclylR¹², heterocyclylR¹², arylR¹², heteroarylR¹², —(C₁₋₉ alkyl)ₙOR⁹, —(C₁₋₉alkyl)ₙSR⁹, —(C₁₋₉alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙNR⁹C(═O)OR⁹, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹;

each A is independently selected from O, S and NR¹¹; and

each n is 0 or 1, or a pharmaceutically acceptable salt or pro-drug thereof.

Another embodiment disclosed herein includes a compound having the structure of formula IV:

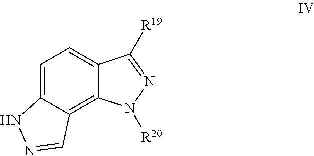

In some embodiments of formula (IV):

each R⁹ is independently selected from the group consisting of H, —C₁₋₉ alkyl, —CF₃, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;

each R¹⁰ is independently selected from the group consisting of —C₁₋₉ alkyl, —CF₃, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;

each R¹¹ is independently selected from the group consisting of CN, —OR⁹ and R⁹;

each R¹² is 1-5 substituents each selected from the group consisting of H, C₁₋₉alkyl, -alkylaminoalkyl, halide, —CF₃, carbocyclylR¹², heterocyclylR¹², arylR¹², heteroarylR¹², —(C₁₋₉ alkyl)ₙOR⁹, —(C₁₋₉alkyl)ₙSR⁹, —(C₁₋₉alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙNR⁹C(═O)OR⁹, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹;

R¹⁹ is independently selected from the group consisting of C₁₋₉alkyl, —OR⁹, amino, —S(═O)R¹⁰, —SO₂R⁹, —N(R⁹)S(═O)R¹⁰, —N(R⁹)SO₂R⁹, —SO₂N(R⁹)₂, —N(R⁹)₂, —N(R⁹)C(═O)N(R⁹)₂, —NR⁹C(═O)OR⁹, —C(═O)N(R⁹)₂, —N(R⁹)C(═O)R⁹, —OC(═O)N(R⁹)₂, —CO₂R⁹ and —C(═O)R⁹;

R²⁰ is independently selected from the group consisting of arylR¹² and heteroarylR¹²;

each A is independently selected from O, S and NR¹¹; and

each n is 0 or 1, or a pharmaceutically acceptable salt or pro-drug thereof.

Other embodiments disclosed herein include methods of inhibiting one or more members of the Wnt pathway, including one or more Wnt proteins by administering to a subject affected by a disorder or disease in which aberrant Wnt signaling is implicated, such as cancer and other diseases associated with abnormal angiogenesis, cellular proliferation, cell cycling and mutations in Wnt signaling components, a compound according to any of the above formulas. Accordingly, the compounds and compositions provided herein can be used to treat cancer, to reduce or inhibit angiogenesis, to reduce or inhibit cellular proliferation and correct an genetic disorder due to mutations in Wnt signaling components. Non-limiting examples of diseases which can be treated with the compounds and compositions provided herein include a variety of cancers, diabetic retinopathy, neovascular glaucoma, rheumatoid arthritis, psoriasis, mycotic and viral infections, osteochondrodysplasia, Alzheimer's disease, osteoarthritis, polyposis coli, osteoporosis-pseudoglioma syndrome, familial exudative vitreoretinopathy, retinal angiogenesis, early coronary disease, tetra-amelia syndrome, Müllerian-duct regression and virilization, SERKAL syndrome, diabetes mellitus type 2, Fuhrmann syndrome, Al-Awadi/Raas-Rothschild/Schinzel phocomelia syndrome, odonto-onycho-dermal dysplasia, obesity, split-hand/foot malformation, caudal duplication syndrome, tooth agenesis, Wilms tumor, skeletal dysplasia, focal dermal hypoplasia, autosomal recessive anonychia, neural tube defects, alpha-thalassemia (ATRX) syndrome, fragile X syndrome, ICF syndrome, Angelman syndrome, Prader-Willi syndrome, Beckwith-Wiedemann Syndrome and Rett syndrome.

Another embodiment disclosed herein includes a pharmaceutical composition that has a compound according to any of the above formulas and a pharmaceutically acceptable carrier, diluent, or excipient.

It is to be understood that both the foregoing general description and the following detailed description are exemplary and explanatory only and are not restrictive of the invention, as claimed.

### DETAILED DESCRIPTION OF THE INVENTION

Compositions and methods for inhibiting one or more members of the Wnt pathway, including one or more Wnt proteins would be of tremendous benefit. Certain embodiments provide such compositions and methods.

Some embodiments relate to a method for treating a disease such as cancers, diabetic retinopathy, neovascular glaucoma, rheumatoid arthritis, psoriasis, mycotic and viral infections, osteochondrodysplasia, Alzheimer's disease, osteoarthritis, polyposis coli, osteoporosis-pseudoglioma syndrome, familial exudative vitreoretinopathy, retinal angiogenesis, early coronary disease, tetra-amelia syndrome, Müllerian-duct regression and virilization, SERKAL syndrome, diabetes mellitus type 2, Fuhrmann syndrome, Al-Awadi/Raas-Rothschild/Schinzel phocomelia syndrome, odonto-onycho-dermal dysplasia, obesity, split-hand/foot malformation, caudal duplication syndrome, tooth agenesis, Wilms tumor, skeletal dysplasia, focal dermal hypoplasia, autosomal recessive anonychia, neural tube defects, alpha-thalassemia (ATRX) syndrome, fragile X syndrome, ICF syndrome, Angelman syndrome, Prader-Willi syndrome, Beckwith-Wiedemann Syndrome and Rett syndrome.

In some embodiments, pharmaceutical compositions are provided that are effective for treatment of a disease of an animal, e.g., a mammal, caused by the pathological activation or mutations of the Wnt pathway. The composition includes a pharmaceutically acceptable carrier and a Wnt pathway inhibitor as described herein.

### DEFINITIONS

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

In this specification and in the claims, the following terms have the meanings as defined. As used herein, “alkyl” means a branched, or straight chain chemical group containing only carbon and hydrogen, such as methyl, isopropyl, isobutyl, sec-butyl and pentyl. Alkyl groups can either be unsubstituted or substituted with one or more substituents, e.g., halogen, alkoxy, acyloxy, amino, amido, cyano, nitro, hydroxyl, mercapto, carboxy, carbonyl, benzyloxy, aryl, heteroaryl, or other functionality that may be suitably blocked, if necessary for purposes of the invention, with a protecting group. Alkyl groups can be saturated or unsaturated (e.g., containing —C═C— or —C≡C— subunits), at one or several positions. Typically, alkyl groups will comprise 1 to 9 carbon atoms, preferably 1 to 6, and more preferably 1 to 4 carbon atoms.

As used herein, “carbocyclyl” means a cyclic ring system containing only carbon atoms in the ring system backbone, such as cyclopropyl, cyclobutyl, cyclopentyl, cyclohexyl, and cyclohexenyl. Carbocyclyls may include multiple fused rings. Carbocyclyls may have any degree of saturation provided that at least one ring in the ring system is not aromatic. Carbocyclyl groups can either be unsubstituted or substituted with one or more substituents, e.g., halogen, alkoxy, acyloxy, amino, amido, cyano, nitro, hydroxyl, mercapto, carboxy, carbonyl, benzyloxy, aryl, heteroaryl, or other functionality that may be suitably blocked, if necessary for purposes of the invention, with a protecting group. Typically, carbocyclyl groups will comprise 3 to 10 carbon atoms, preferably 3 to 6.

As used herein, “lower alkyl” means a subset of alkyl, and thus is a hydrocarbon substituent, which is linear, or branched. Preferred lower alkyls are of 1 to about 4 carbons, and may be branched or linear. Examples of lower alkyl include butyl, propyl, isopropyl, ethyl, and methyl. Likewise, radicals using the terminology “lower” refer to radicals preferably with 1 to about 4 carbons in the alkyl portion of the radical.

As used herein, “amido” means a H—CON— or alkyl-CON—, carbocyclyl-CON—, aryl-CON—, heteroaryl-CON— or heterocyclyl-CON group wherein the alkyl, carbocyclyl, aryl or heterocyclyl group is as herein described.

As used herein, “aryl” means an aromatic radical having a single-ring (e.g., phenyl) or multiple condensed rings (e.g., naphthyl or anthryl) with only carbon atoms present in the ring backbone. Aryl groups can either be unsubstituted or substituted with one or more substituents, e.g., amino, cyano, hydroxyl, lower alkyl, haloalkyl, alkoxy, nitro, halo, mercapto, and other substituents. A preferred carbocyclic aryl is phenyl.

As used herein, the term “heteroaryl” means an aromatic radical having one or more heteroatom(s) (e.g., N, O, or S) in the ring backbone and may include a single ring (e.g., pyridine) or multiple condensed rings (e.g., quinoline). Heteroaryl groups can either be unsubstituted or substituted with one or more substituents, e.g., amino, cyano, hydroxyl, lower alkyl, haloalkyl, alkoxy, nitro, halo, mercapto, and other substituents. Examples of heteroaryl include thienyl, pyrridyl, furyl, oxazolyl, oxadiazolyl, pyrollyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl and others.

In these definitions it is clearly contemplated that substitution on the aryl and heteroaryl rings is within the scope of certain embodiments. Where substitution occurs, the radical is called substituted aryl or substituted heteroaryl. Preferably one to three and more preferably one or two substituents occur on the aryl ring. Though many substituents will be useful, preferred substituents include those commonly found in aryl compounds, such as alkyl, cycloalkyl, hydroxy, alkoxy, cyano, halo, haloalkyl, mercapto and the like.

As used herein, “amide” includes both RNR′CO— (in the case of R=alkyl, alkaminocarbonyl-) and RCONR′— (in the case of R=alkyl, alkyl carbonylamino-).

As used herein, the term “ester” includes both ROCO— (in the case of R=alkyl, alkoxycarbonyl-) and RCOO— (in the case of R=alkyl, alkylcarbonyloxy-).

As used herein, “acyl” means an H—CO— or alkyl-CO—, carbocyclyl-CO—, aryl-CO—, heteroaryl-CO— or heterocyclyl-CO— group wherein the alkyl, carbocyclyl, aryl or heterocyclyl group is as herein described. Preferred acyls contain a lower alkyl. Exemplary alkyl acyl groups include formyl, acetyl, propanoyl, 2-methylpropanoyl, t-butylacetyl, butanoyl and palmitoyl.

As used herein, “halo or halide” is a chloro, bromo, fluoro or iodo atom radical. Chloro, bromo and fluoro are preferred halides. The term “halo” also contemplates terms sometimes referred to as “halogen”, or “halide”.

As used herein, “haloalkyl” means a hydrocarbon substituent, which is linear or branched or cyclic alkyl, alkenyl or alkynyl substituted with chloro, bromo, fluoro or iodo atom(s). Most preferred of these are fluoroalkyls, wherein one or more of the hydrogen atoms have been substituted by fluoro. Preferred haloalkyls are of 1 to about 3 carbons in length, more preferred haloalkyls are 1 to about 2 carbons, and most preferred are 1 carbon in length. The skilled artisan will recognize then that as used herein, “haloalkylene” means a diradical variant of haloalkyl, such diradicals may act as spacers between radicals, other atoms, or between the parent ring and another functional group.

As used herein, “heterocyclyl” means a cyclic ring system comprising at least one heteroatom in the ring system backbone. Heterocyclyls may include multiple fused rings. Heterocyclyls may have any degree of saturation provided that at least one ring in the ring system is not aromatic. Heterocyclyls may be substituted or unsubstituted with one or more substituents, e.g., halogen, alkoxy, acyloxy, amino, amido, cyano, nitro, hydroxyl, mercapto, carboxy, carbonyl, benzyloxy, aryl, heteroaryl, and other substituents, and are attached to other groups via any available valence, preferably any available carbon or nitrogen. More preferred heterocycles are of 5-7 members. In six membered monocyclic heterocycles, the heteroatom(s) are selected from one up to three of O, N or S, and wherein when the heterocycle is five membered, preferably it has one or two heteroatoms selected from O, N, or S.

As used herein, “substituted amino” means an amino radical which is substituted by one or two alkyl, cycloalkyl, aryl, heteroaryl or heterocyclyl groups, wherein the alkyl, aryl, heteroaryl or heterocyclyl are defined as above.

As used herein, “substituted thiol” means RS— group wherein R is an alkyl, an aryl, heteroaryl or a heterocyclyl group, wherein the alkyl, cycloalkyl, aryl, heteroaryl or heterocyclyl are defined as above.

As used herein, “sulfonyl” means an alkylSO₂, arylSO₂₅ heteroarylSO₂, carbocyclylSO₂, or heterocyclyl-SO₂ group wherein the alkyl, carbocyclyl, aryl, heteroaryl or heterocyclyl are defined as above.

As used herein, “sulfamido” means an alkyl-N—S(O)₂N—, aryl-NS(O)₂N—, heteroaryl-NS(O)₂N—, carbocyclyl-NS(O)₂N or heterocyclyl-NS(O)₂N— group wherein the alkyl, carbocyclyl, aryl, heteroaryl or heterocyclyl group is as herein described.

As used herein, “sulfonamido” means an alkyl-S(O)₂N—, aryl-S(O)₂N—, heteroaryl-S(O)₂N—, carbocyclyl-S(O)₂N— or heterocyclyl-S(O)₂N— group wherein the alkyl, carbocyclyl, aryl, heteroaryl or heterocyclyl group is as herein described.

As used herein, “ureido” means an alkyl-NCON—, aryl-NCON—, heteroaryl-NCON—, carbocyclyl-NCON— or heterocyclyl-NCON— group wherein the alkyl, carbocyclyl, aryl, heteroaryl or heterocyclyl group is as herein described.

As used herein, when two groups are indicated to be “linked” or “bonded” to form a “ring,” it is to be understood that a bond is formed between the two groups and may involve replacement of a hydrogen atom on one or both groups with the bond, thereby forming a carbocyclyl, heterocyclyl, aryl, or heteroaryl ring. The skilled artisan will recognize that such rings can and are readily formed by routine chemical reactions, and it is within the purview of the skilled artisan to both envision such rings and the methods of their formations. Preferred are rings having from 3-7 members, more preferably 5 or 6 members. As used herein the term “ring” or “rings” when formed by the combination of two radicals refers to heterocyclic, carbocyclic, aryl, or heteroaryl rings.

The skilled artisan will recognize that some structures described herein may be resonance forms or tautomers of compounds that may be fairly represented by other chemical structures, even when kinetically, the artisan recognizes that such structures are only a very small portion of a sample of such compound(s). Such compounds are clearly contemplated within the scope of this invention, though such resonance forms or tautomers are not represented herein.

The compounds provided herein may encompass various stereochemical forms. The compounds also encompasses diastereomers as well as optical isomers, e.g. mixtures of enantiomers including racemic mixtures, as well as individual enantiomers and diastereomers, which arise as a consequence of structural asymmetry in certain compounds. Separation of the individual isomers or selective synthesis of the individual isomers is accomplished by application of various methods which are well known to practitioners in the art. Unless otherwise indicated, when a disclosed compound is named or depicted by a structure without specifying the stereochemistry and has one or more chiral centers, it is understood to represent all possible stereoisomers of the compound.

The term “administration” or “administering” refers to a method of giving a dosage of a compound or pharmaceutical composition to a vertebrate or invertebrate, including a mammal, a bird, a fish, or an amphibian, where the method is, e.g., intrarespiratory, topical, oral, intravenous, intraperitoneal, intramuscular, buccal, rectal, sublingual. The preferred method of administration can vary depending on various factors, e.g., the components of the pharmaceutical composition, the site of the disease, the disease involved, and the severity of the disease.

A “diagnostic” as used herein is a compound, method, system, or device that assists in the identification and characterization of a health or disease state. The diagnostic can be used in standard assays as is known in the art.

The term “mammal” is used in its usual biological sense. Thus, it specifically includes humans, cattle, horses, dogs, and cats, but also includes many other species.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (2006); Goodman and Gilman's: The Pharmacological Basis of Therapeutics, 11th Ed., The McGraw-Hill Companies.

The term “pharmaceutically acceptable salt” refers to salts that retain the biological effectiveness and properties of the compounds of the preferred embodiments and, which are not biologically or otherwise undesirable. In many cases, the compounds of the preferred embodiments are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in World Patent Publication 87/05297, Johnston et al., published Sep. 11, 1987 (incorporated by reference herein).

“Solvate” refers to the compound formed by the interaction of a solvent and a Wnt pathway inhibitor, a metabolite, or salt thereof. Suitable solvates are pharmaceutically acceptable solvates including hydrates.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

By “therapeutically effective amount” or “pharmaceutically effective amount” is typically one which is sufficient to achieve the desired effect and may vary according to the nature and severity of the disease condition, and the potency of the compound. It will be appreciated that different concentrations may be employed for prophylaxis than for treatment of an active disease. This amount can further depend upon the patient's height, weight, sex, age and medical history.

A therapeutic effect relieves, to some extent, one or more of the symptoms of the disease, and includes curing a disease. “Curing” means that the symptoms of active disease are eliminated. However, certain long-term or permanent effects of the disease may exist even after a cure is obtained (such as extensive tissue damage).

“Treat,” “treatment,” or “treating,” as used herein refers to administering a pharmaceutical composition for therapeutic purposes. The term “therapeutic treatment” refers to administering treatment to a patient already suffering from a disease thus causing a therapeutically beneficial effect, such as ameliorating existing symptoms, preventing additional symptoms, ameliorating or preventing the underlying metabolic causes of symptoms, postponing or preventing the further development of a disorder and/or reducing the severity of symptoms that will or are expected to develop.

Compounds

The compounds and compositions described herein can be used as anti-proliferative agents, e.g., anti-cancer and anti-angiogenesis agents, and as inhibitors of the Wnt signaling pathway, e.g., for treating diseases or disorders associated with aberrant Wnt signaling. In addition, the compounds can be used as inhibitors of one or more kinases, kinase receptors, or kinase complexes (e.g., VEGF, CHK-1, CLK, HIPK, Abl, JAK and/or CDK complexes). Such compounds and compositions are also useful for controlling cellular proliferation, differentiation, and/or apoptosis.

Some embodiments of the present invention include compounds, salts, pharmaceutically acceptable salts or pro-drugs thereof of formula (I):

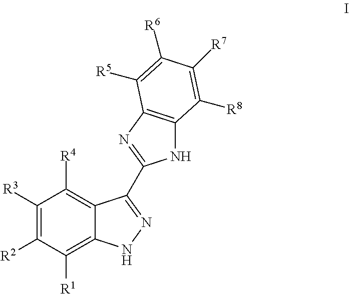

In some embodiments, R¹, R², R⁴, R⁵, R⁶, R⁷ and R⁸ are independently selected from the group consisting of H, C₁₋₉ alkyl, halide, —CF₃, —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)S(═O)R¹⁰, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹.

In some embodiments, R³ is selected from the group consisting of —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)S(═O)R¹⁰, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹.

In some embodiments, one of each R¹ and R², R² and R³, R³ and R⁴, R⁵ and R⁶, R⁶ and R⁷ or R⁷ and R⁸ are taken together to form a ring which is selected from the group consisting of aryl, heteroaryl,

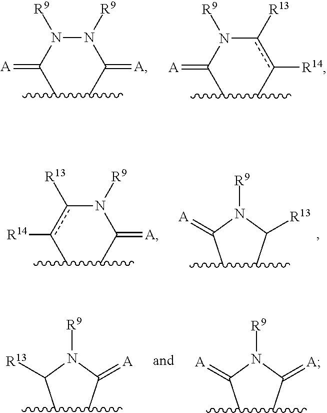

wherein each bond represented by a dashed and solid line represents a bond selected from the group consisting of a single bond and a double bond.

In some embodiments, each R⁹ is independently selected from the group consisting of H, —C₁₋₉ alkyl, —CF₃, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹².

In some embodiments, each R¹⁰ is independently selected from the group consisting of —C₁₋₉ alkyl, —CF₃, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹².

In some embodiments, each R¹¹ is independently selected from the group consisting of CN, —OR⁹ and R⁹.

In some embodiments, each R¹² is 1-5 substituents each selected from the group consisting of H, C₁₋₉ alkyl, -alkylaminoalkyl, halide, —CF₃, carbocyclylR¹², heterocyclylR¹², arylR¹², heteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙNR⁹C(═O)OR⁹, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹.

In some embodiments, R¹³ and R¹⁴ are independently selected from the group consisting of H, C₁₋₉ alkyl, halide, —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙN(R⁹)C(=A)R⁹, —NO₂, —CN, —(C₁₋₉ alkyl)ₙCO₂R⁹ and —(C₁₋₉ alkyl)ₙC(=A)R⁹.

In some embodiments, R¹³ and R¹⁴ are taken together to form a ring which is selected from the group consisting of benzene and pyridine.

In some embodiments, each A is independently selected from O, S and NR¹¹.

In some embodiments, each n is independently 0 or 1.

Illustrative compounds of Formula (I) are shown in Table 1.

TABLE 1

| | |
|---|---|
| 1 | 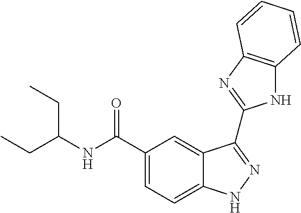 |
| 2 | 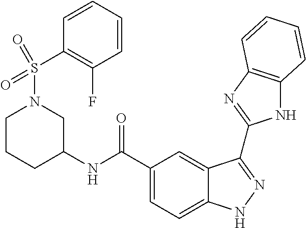 |
| 3 | 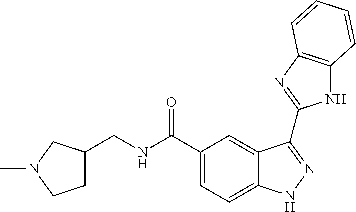 |
| 4 | 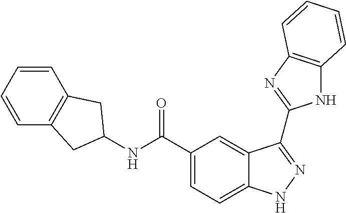 |
| 5 | 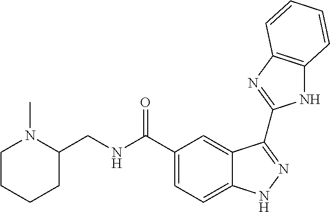 |
| 6 | 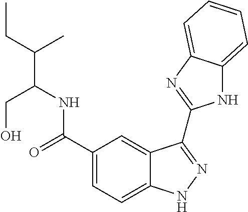 |
| 7 | 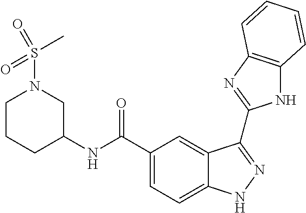 |
| 8 | 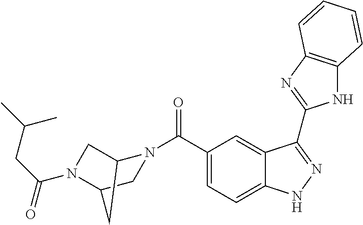 |
| 9 | 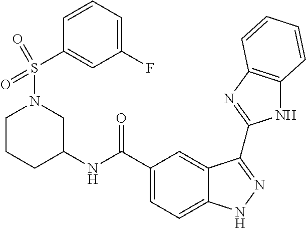 |
| 10 | 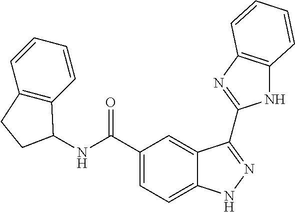 |
| 11 | 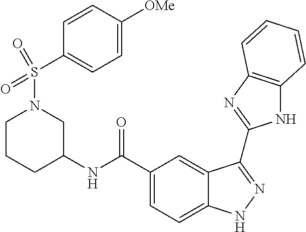 |
| 12 | 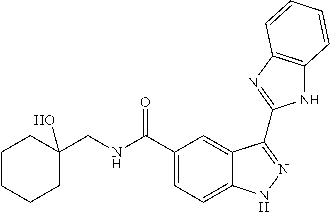 |
| 13 | 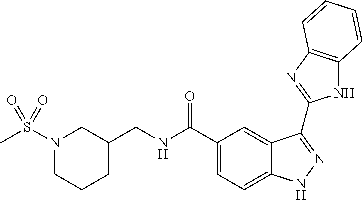 |
| 14 | 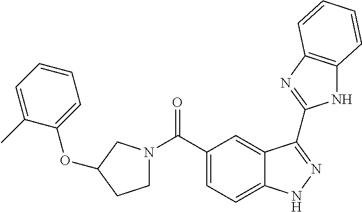 |
| 15 | 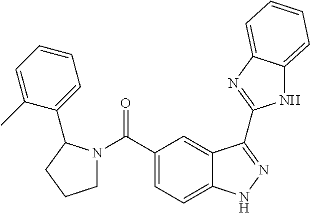 |
| 16 | 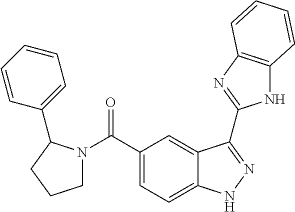 |
| 17 | 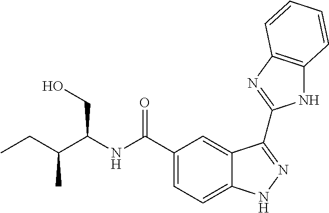 |
| 18 | 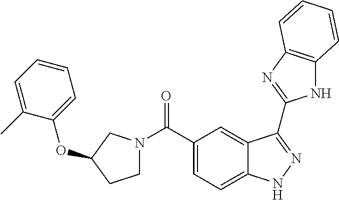 |
| 19 | 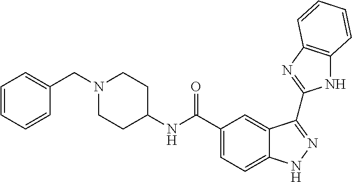 |
| 20 | 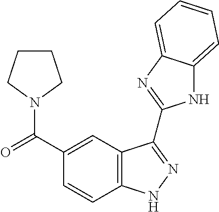 |
| 21 | 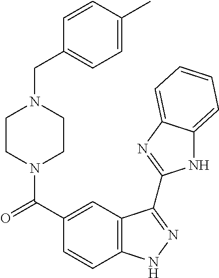 |
| 22 | 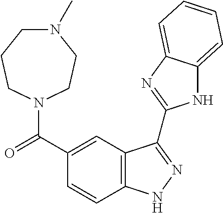 |
| 23 | 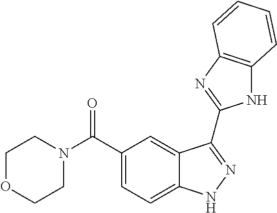 |
| 24 | 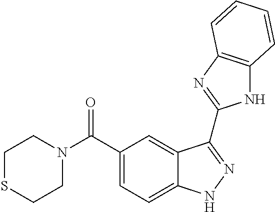 |
| 25 | 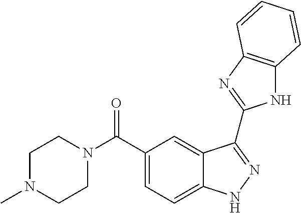 |
| 26 | 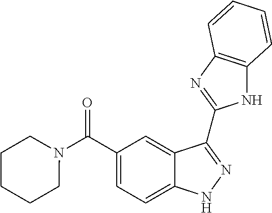 |
| 27 | 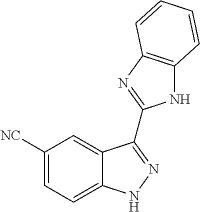 |
| 28 | 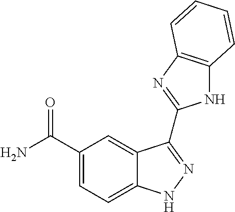 |
| 29 | 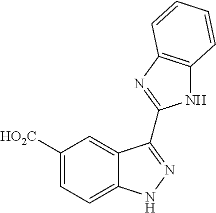 |
| 30 | 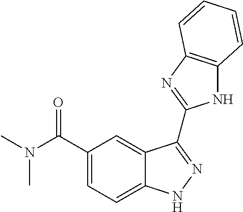 |
| 31 | 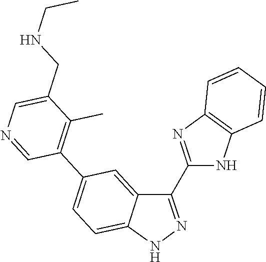 |
| 32 | 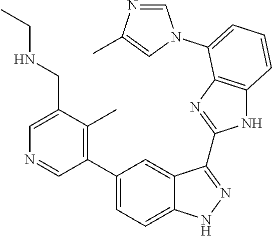 |
| 33 | 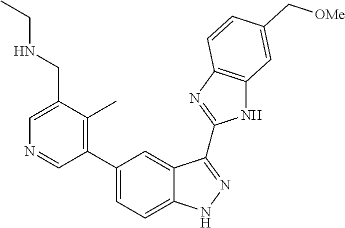 |
| 34 | 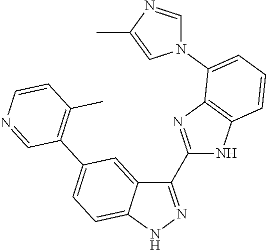 |
| 35 | 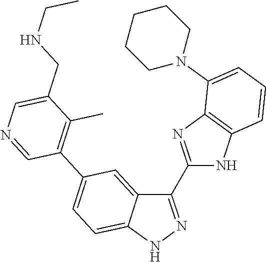 |
| 36 | 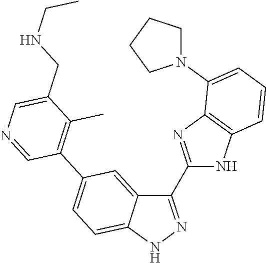 |
| 37 | 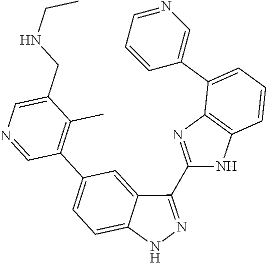 |
| 38 | 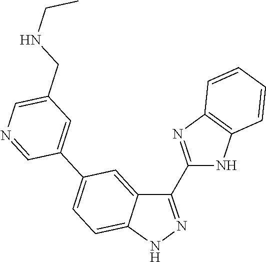 |
| 39 | 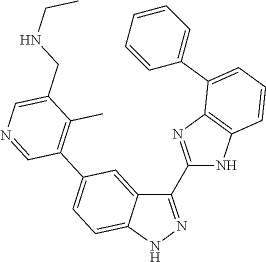 |
| 40 | 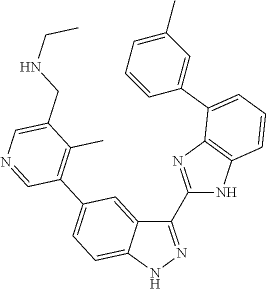 |
| 41 | 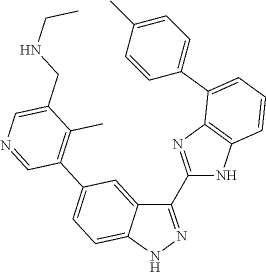 |
| 42 | 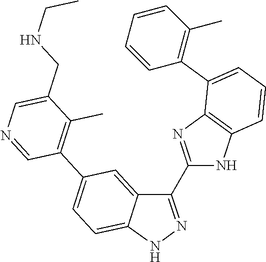 |
| 43 | 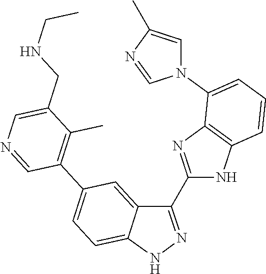 |
| 44 | 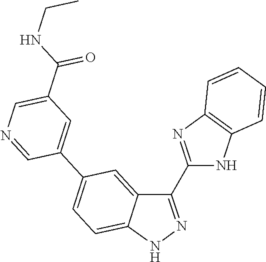 |
| 45 | 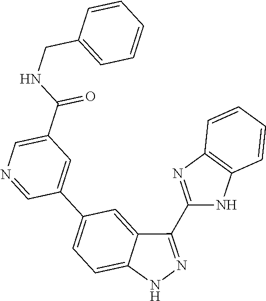 |
| 46 | 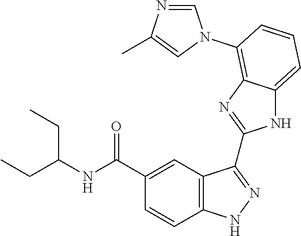 |

Some embodiments of the present invention include compounds, salts, pharmaceutically acceptable salts or pro-drugs thereof of formula (II):

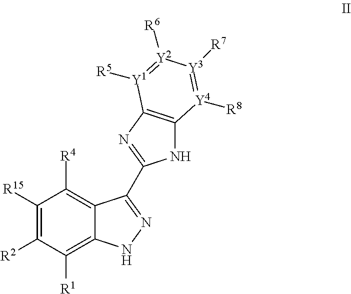

In some embodiments, R¹, R², R⁴, R⁵, R⁶, R⁷ and R⁸ are independently selected from the group consisting of H, C₁₋₉ alkyl, halide, —CF₃, —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)S(═O)R¹⁰, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹.

In some embodiments, one of each R¹ and R², R² and R¹⁵, R¹⁵ and R⁴, R⁵ and R⁶, R⁶ and R⁷ or R⁷ and R⁸ are taken together to form a ring which is selected from the group consisting of aryl, heteroaryl,

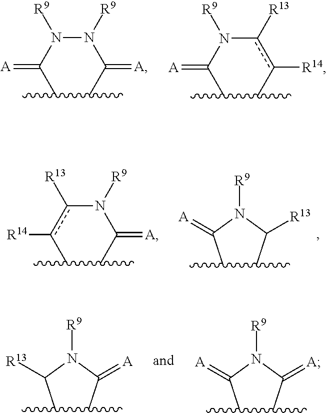

wherein each bond represented by a dashed and solid line represents a bond selected from the group consisting of a single bond and a double bond.

In some embodiments, each R⁹ is independently selected from the group consisting of H, —C₁₋₉ alkyl, —CF₃, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹².

In some embodiments, each R¹⁰ is independently selected from the group consisting of —C₁₋₉ alkyl, —CF₃, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹².

In some embodiments, each R¹¹ is independently selected from the group consisting of CN, —OR⁹ and R⁹.

In some embodiments, each R¹² is 1-5 substituents each selected from the group consisting of H, C₁₋₉ alkyl, -alkylaminoalkyl, halide, —CF₃, carbocyclylR¹², heterocyclylR¹², arylR¹², heteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂₅—(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙNR⁹C(═O)OR⁹, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹.

In some embodiments, R¹³ and R¹⁴ are independently selected from the group consisting of H, C₁₋₉ alkyl, halide, —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙN(R⁹)C(=A)R⁹, —NO₂, —CN, —(C₁₋₉ alkyl)ₙCO₂R⁹ and —(C₁₋₉ alkyl)ₙC(=A)R⁹.

In some embodiments, R¹³ and R¹⁴ are taken together to form a ring which is selected from the group consisting of benzene and pyridine.

In some embodiments, each R¹⁵ is selected from the group consisting of -carbocyclylR¹⁶, —(C₁₋₉ alkyl)carbocyclylR¹², -heterocyclylR¹⁶, —(C₁₋₉ alkyl)heterocyclylR¹², -arylR¹⁶, —(C₁₋₉ alkyl)arylR¹², -heteroarylR¹⁶, —(C₁₋₉ alkyl)heteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)N(R⁹)S(═O)R¹⁰, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, —NO₂, —CN, —(C₁₋₉ alkyl)ₙCO₂R⁹ and —(C₁₋₉ alkyl)ₙC(=A)R⁹.

In some embodiments, each R¹⁶ is 1-5 substituents each selected from the group consisting of H, C₁₋₉ alkyl, halide, —CF₃, carbocyclylR¹², heterocyclylR¹², arylR¹², heteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)N(R⁹)SO₂R⁹, —(C₁₋₉ alkyl)ₙSO₂N(R⁹)₂, —N(R¹⁷)₂, —(C₁₋₉ alkyl)N(R⁹)₂, —NR⁹C(═O)N(R¹⁷)₂, —(C₁₋₉ alkyl)N(R⁹)C(=A)N(R⁹)₂, —C(═O)NR⁹R¹⁸, —(C₁₋₉ alkyl)C(=A)N(R⁹)₂, —(C₁₋₉ alkyl)NR⁹C(═O)OR⁹, —(C₁₋₉ alkyl)N(R⁹)C(=A)R⁹, —(C₁₋₉ alkyl)OC(═O)N(R⁹)₂, —NO₂, —CN, —(C₁₋₉ alkyl)ₙCO₂R⁹ and —(C₁₋₉ alkyl)C(=A)R⁹.

In some embodiments, each R¹⁷ is H, —(C₁₋₉ alkyl)ₙcarbocyclyl, —(C₁₋₉ alkyl)ₙheterocyclyl, —(C₁₋₉ alkyl)ₙaryl and —(C₁₋₉ alkyl)ₙheteroaryl.

In some embodiments, each R¹⁸ is independently selected from the group consisting of H, —C₁₋₉ alkyl, —CF₃, -carbocyclylR¹², —(C₁₋₉ alkyl)ₙarylR¹² and —(C₁₋₉ alkyl)ₙheteroarylR¹².

In some embodiments, each A is independently selected from O, S and NR¹¹.

In some embodiments, Y¹, Y², Y³ and Y⁴ are independently selected from the group consisting of carbon and nitrogen with the proviso that at least one of Y¹, Y², Y³ and Y⁴ are nitrogen.

In some embodiments, Y¹ is nitrogen and R⁵ is absent.

In some embodiments, Y² is nitrogen and R⁶ is absent.

In some embodiments, Y³ is nitrogen and R⁷ is absent.

In some embodiments, Y⁴ is nitrogen and R⁸ is absent.

In some embodiments, each n is 0 or 1.

Illustrative compounds of Formula (II) are shown in Table 2.

TABLE 2

| | |
|---|---|
| 47 | 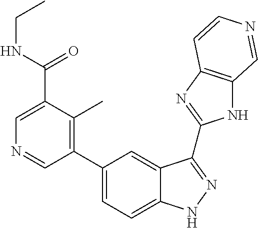 |

Some embodiments of the present invention include compounds, salts, pharmaceutically acceptable salts or pro-drugs thereof of formula (III):

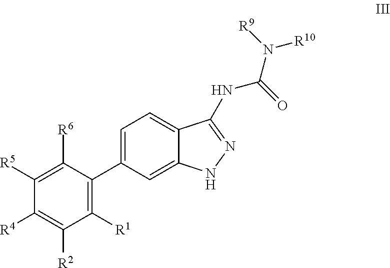

In some embodiments, R¹, R², R⁴, R⁵ and R⁶ are independently selected from the group consisting of H, C₁₋₉ alkyl, halide, —CF₃, —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)S(═O)R¹⁰, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹.

In some embodiments, each R⁹ is independently selected from the group consisting of H, —C₁₋₉ alkyl, —(C₁₋₉ alkyl)OR^{o}, —(C₁₋₉ alkyl)N(R⁹)₂, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;

In some embodiments, each R¹⁰ is independently selected from the group consisting of —C₁₋₉ alkyl, —(C₁₋₉ alkyl)OR⁹, —(C₁₋₉ alkyl)N(R⁹)₂, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;

In some embodiments, R⁹ and R¹⁰ are taken together to form a 3-10 membered heterocyclyl ring.

In some embodiments, each R¹¹ is independently selected from the group consisting of CN, —OR⁹ and R⁹.

In some embodiments, each R¹² is 1-5 substituents each selected from the group consisting of H, C₁₋₉alkyl, -alkylaminoalkyl, halide, —CF₃₅ carbocyclylR¹², heterocyclylR¹², arylR¹², heteroarylR¹², —(C₁₋₉ alkyl)ₙOR⁹, —(C₁₋₉alkyl)ₙSR⁹, —(Cᵢ-9alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂₅—(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙNR⁹C(═O)OR⁹, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹.

In some embodiments, each A is independently selected from O, S and NR¹¹.

In some embodiments, each n is 0 or 1.

Illustrative compounds of Formula (III) are shown in Table 3.

TABLE 3

| | |
|---|---|
| 48 | 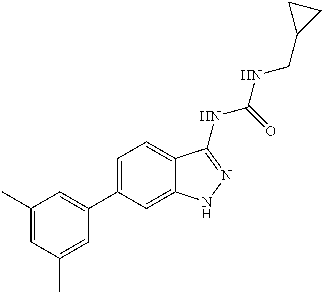 |
| 49 | 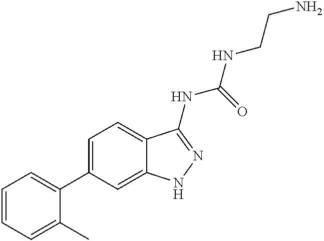 |
| 50 | 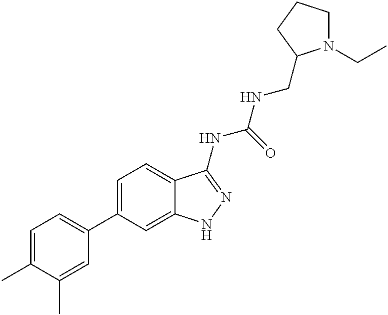 |
| 51 | 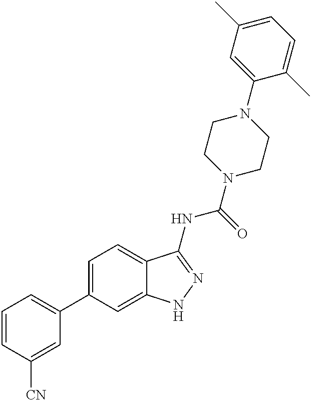 |
| 52 | 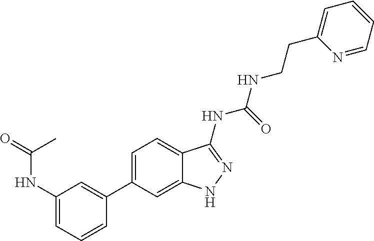 |
| 53 | 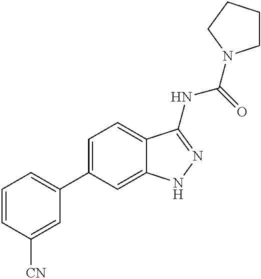 |
| 54 | 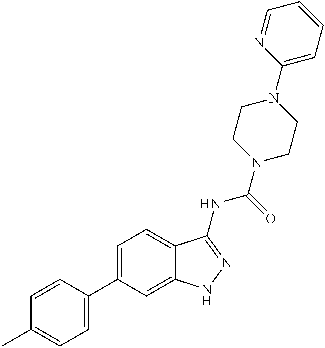 |
| 55 | 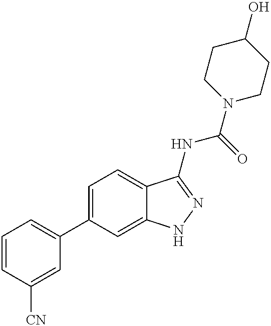 |
| 56 | 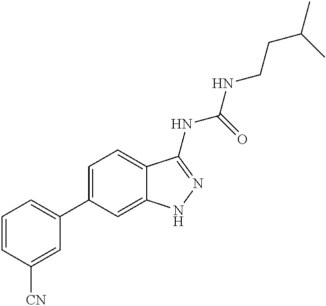 |
| 57 | 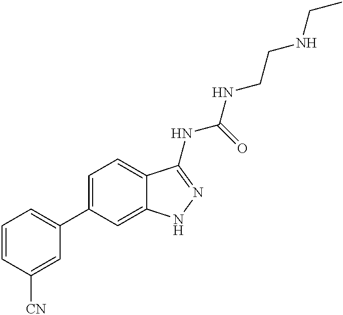 |
| 58 | 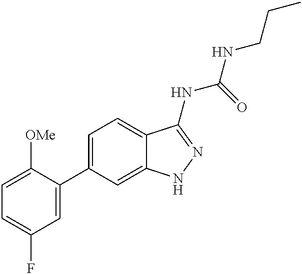 |
| 59 | 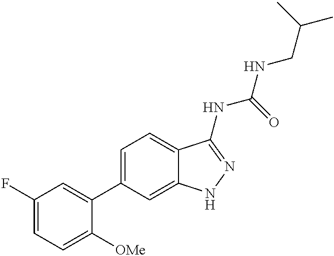 |
| 60 | 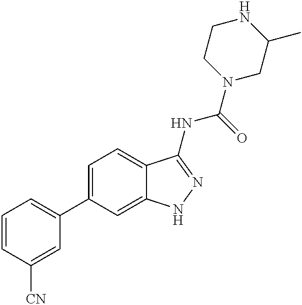 |
| 61 | 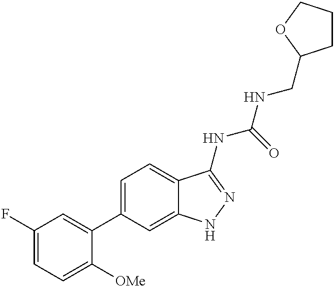 |
| 62 | 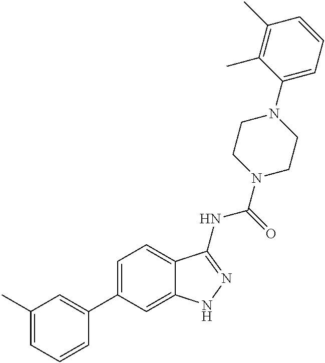 |
| 63 | 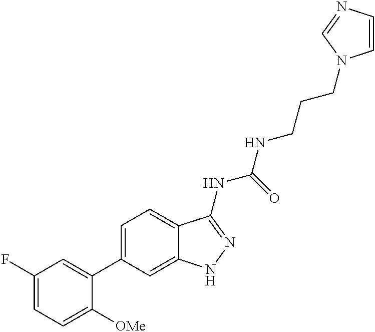 |
| 64 | 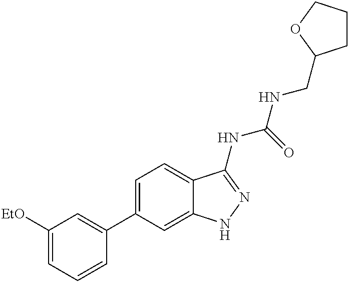 |
| 65 | 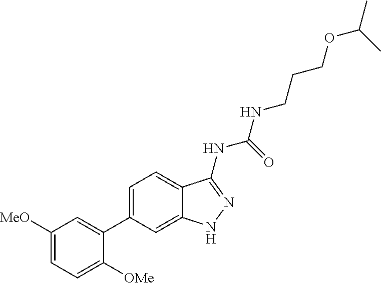 |
| 66 | 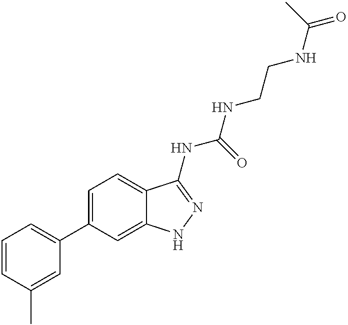 |
| 67 | 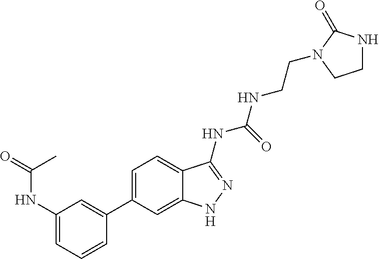 |
| 68 | 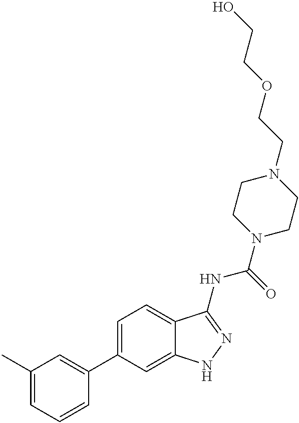 |
| 69 | 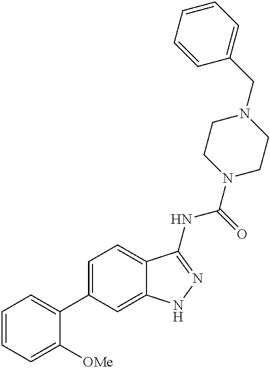 |
| 70 | 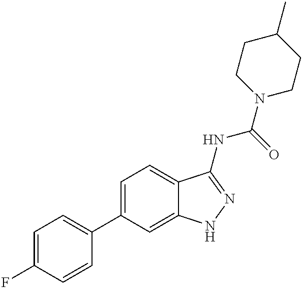 |
| 71 | 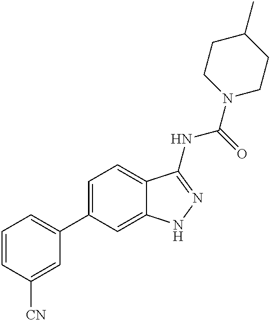 |
| 72 | 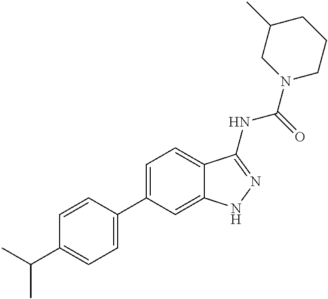 |
| 73 | 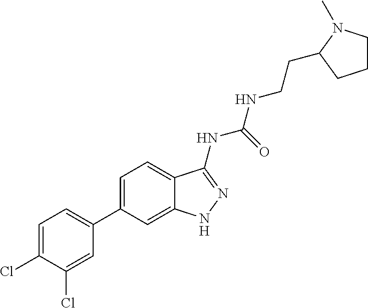 |
| 74 | 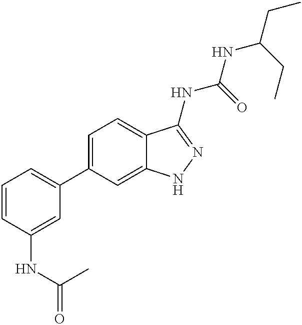 |
| 75 | 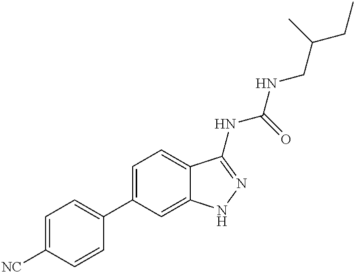 |
| 76 | 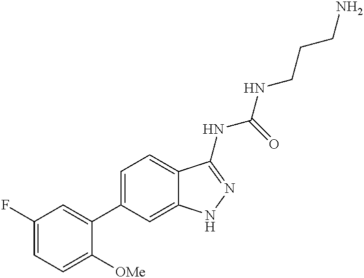 |
| 77 | 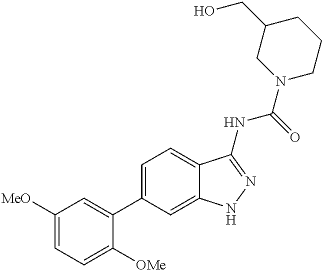 |
| 78 | 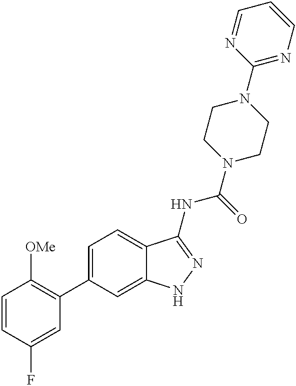 |
| 79 | 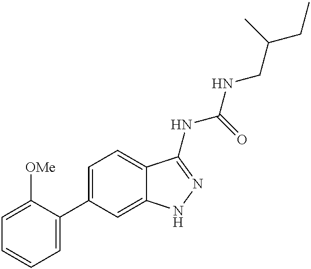 |
| 80 | 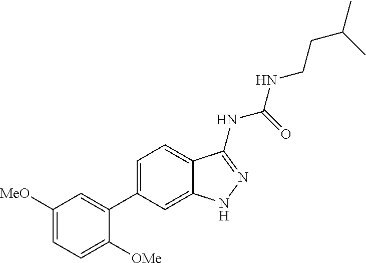 |
| 81 | 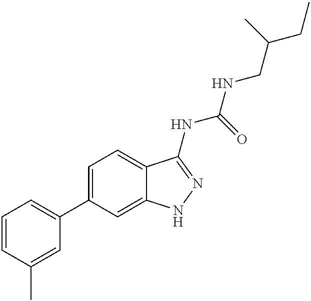 |
| 82 | 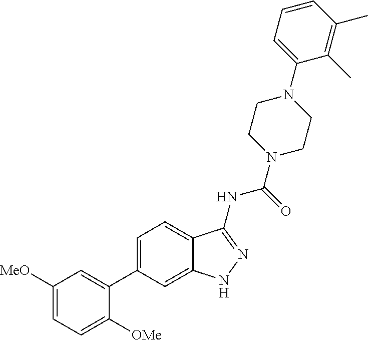 |
| 83 | 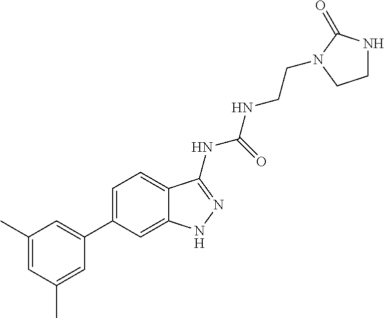 |
| 84 | 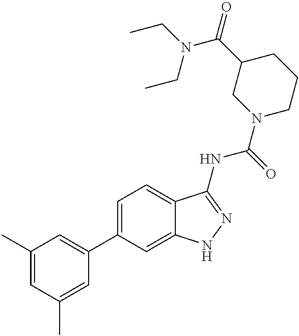 |
| 85 | 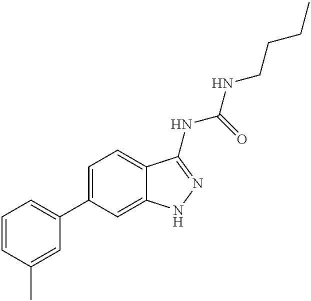 |
| 86 | 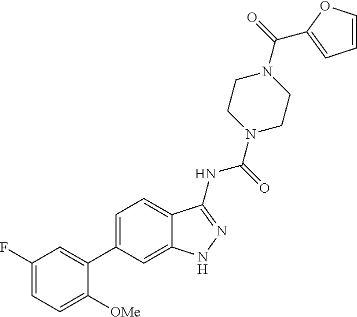 |
| 87 | 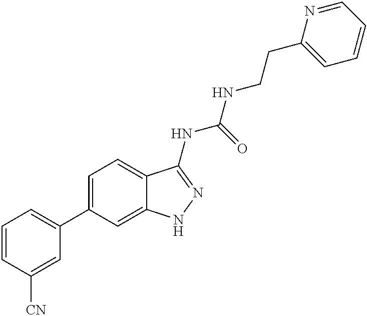 |
| 88 | 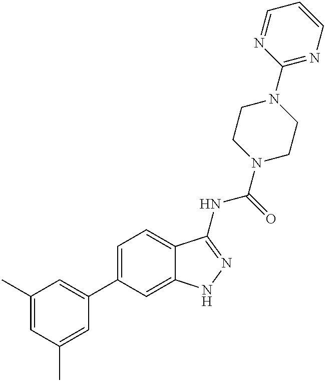 |
| 89 | 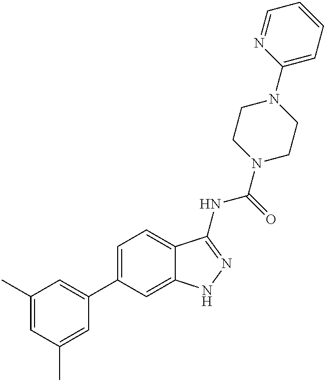 |
| 90 | 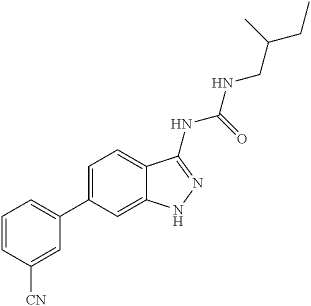 |
| 91 | 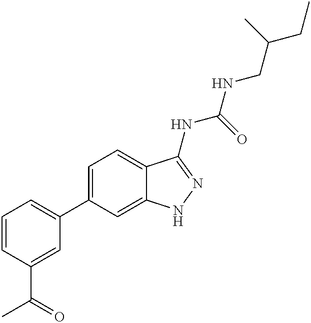 |
| 92 | 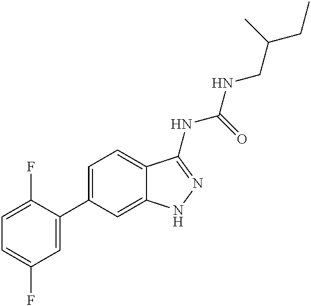 |
| 93 | 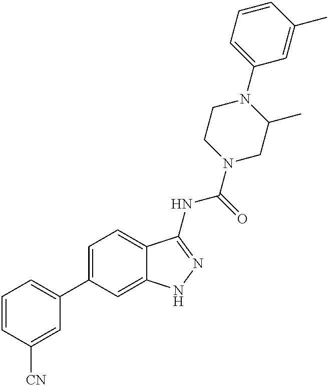 |
| 94 | 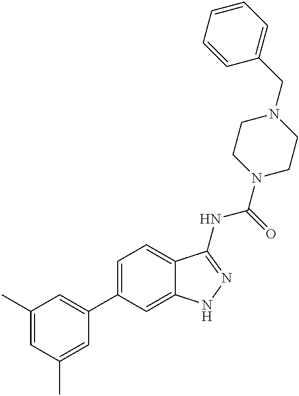 |
| 95 | 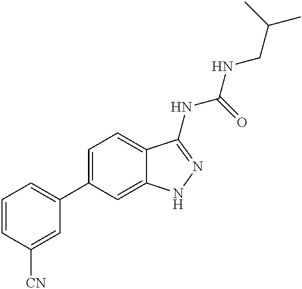 |
| 96 | 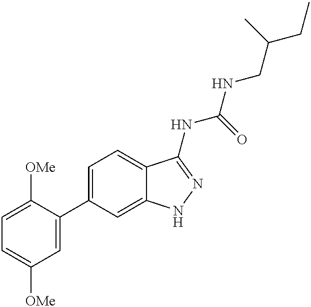 |
| 97 | 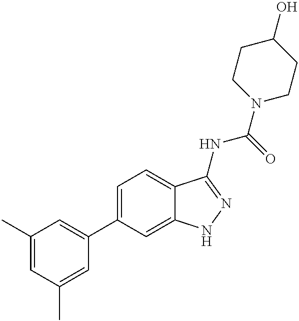 |
| 98 | 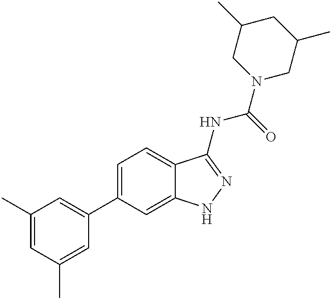 |
| 99 | 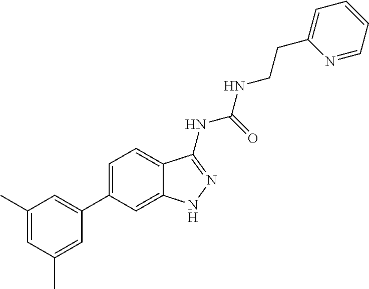 |
| 100 | 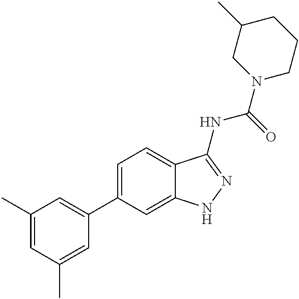 |
| 101 | 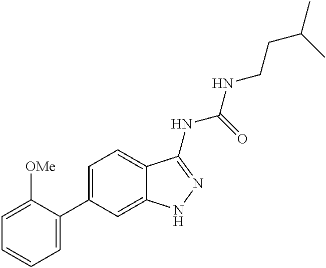 |
| 102 | 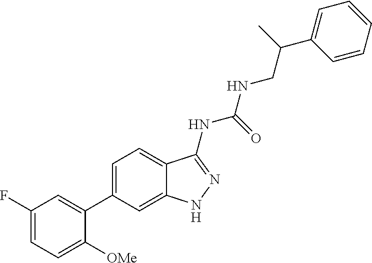 |
| 103 | 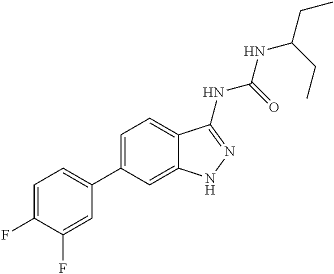 |
| 104 | 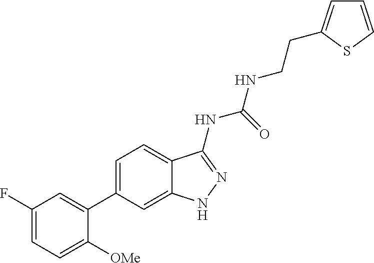 |
| 105 | 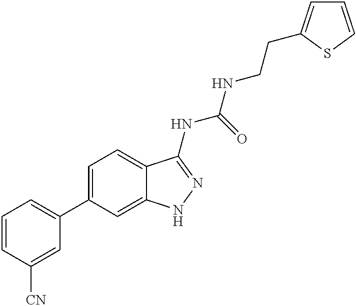 |
| 106 | 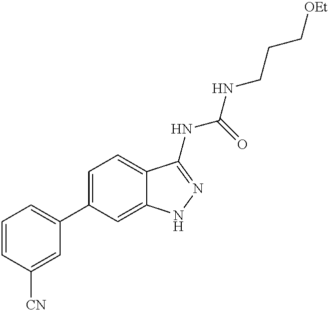 |
| 107 | 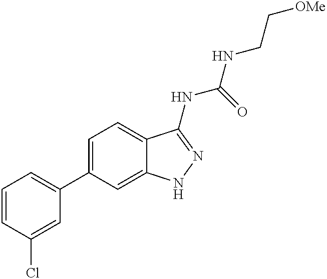 |
| 108 | 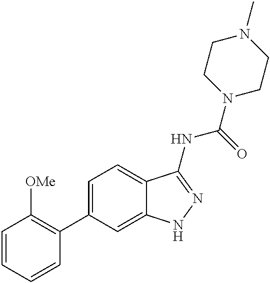 |
| 109 | 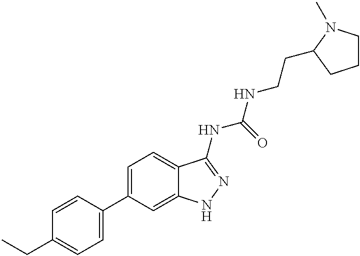 |
| 110 | 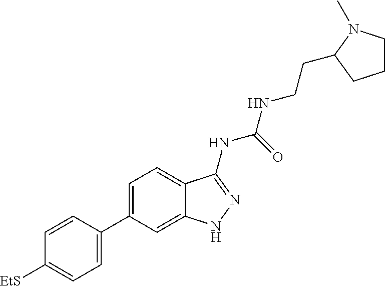 |
| 111 | 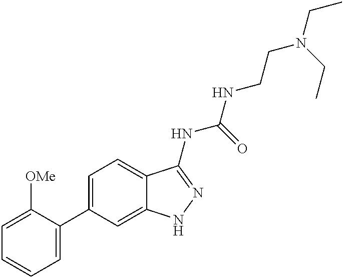 |
| 112 | 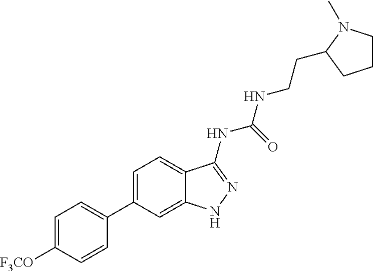 |
| 113 | 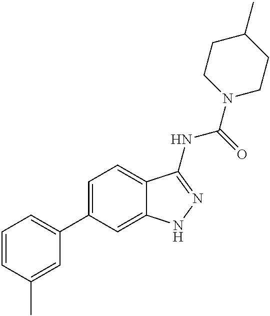 |
| 114 | 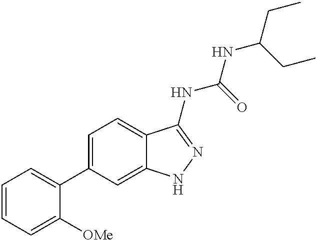 |
| 115 | 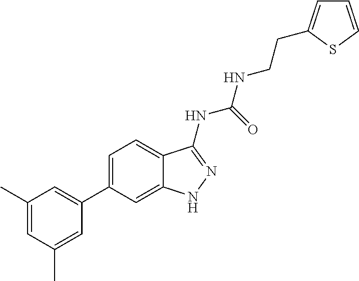 |
| 116 | 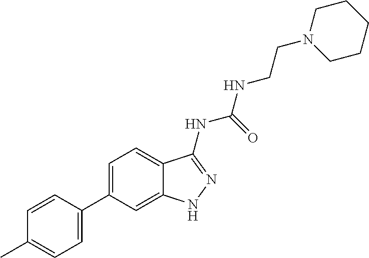 |
| 117 | 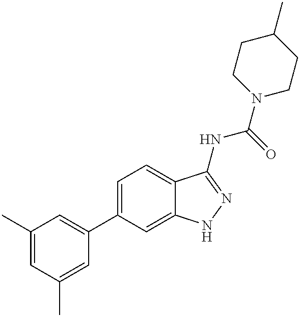 |
| 118 | 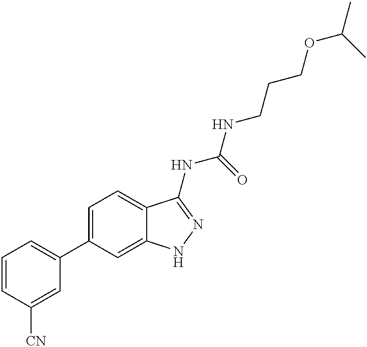 |
| 119 | 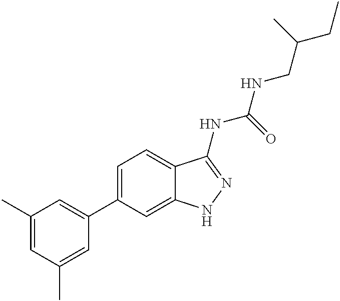 |
| 120 | 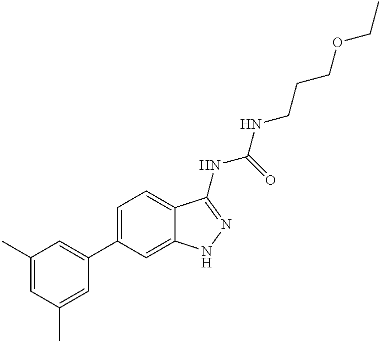 |
| 121 | 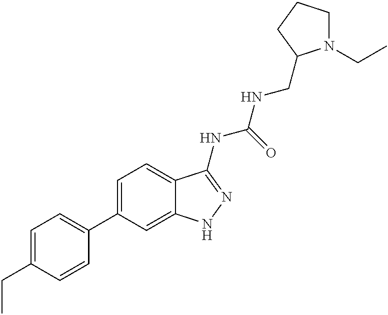 |
| 122 | 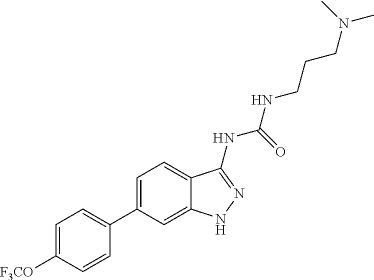 |

Some embodiments of the present invention include compounds, salts, pharmaceutically acceptable salts or pro-drugs thereof of formula (IV):

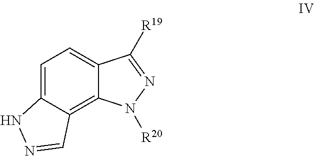

In some embodiments, each R⁹ is independently selected from the group consisting of H, —C₁₋₉ alkyl, —CF₃₅—(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹².

In some embodiments, each R¹⁰ is independently selected from the group consisting of —C₁₋₉ alkyl, —CF₃₅—(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹².

In some embodiments, each R¹¹ is independently selected from the group consisting of CN, —OR⁹ and R⁹.

In some embodiments, each R¹² is 1-5 substituents each selected from the group consisting of H, C₁₋₉alkyl, -alkylaminoalkyl, halide, —CF₃₅ carbocyclylR¹², heterocyclylR¹², arylR¹², heteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂₅—(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙNR⁹C(═O)OR⁹, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹.

In some embodiments, R¹⁹ is independently selected from the group consisting of C₁₋₉alkyl, —OR⁹, amino, —S(═O)R¹⁰, —SO₂R⁹, —N(R⁹)S(═O)R¹⁰, —N(R⁹)SO₂R⁹, —SO₂N(R⁹)₂, —N(R⁹)₂, —N(R⁹)C(═O)N(R⁹)₂, —NR⁹C(═O)OR⁹, —C(═O)N(R⁹)₂, —N(R⁹)C(═O)R⁹, —OC(═O)N(R⁹)₂, —CO₂R⁹ and —C(═O)R⁹.

In some embodiments, R²⁰ is independently selected from the group consisting of arylR¹² and heteroarylR¹².

In some embodiments, each A is independently selected from O, S and NR¹¹.

In some embodiments, each n is 0 or 1.

Illustrative compounds of Formula (IV) are shown in Table 4.

TABLE 4

| | |
|---|---|
| 123 | 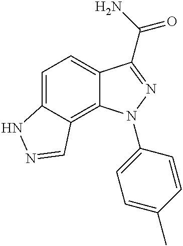 |
| 124 | 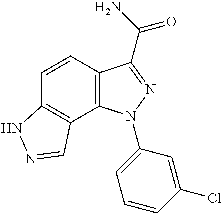 |
| 125 | 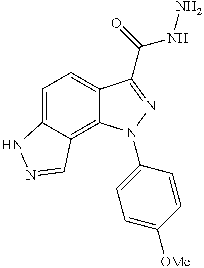 |
| 126 | 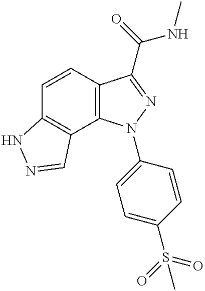 |
| 127 | 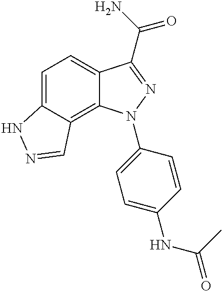 |
| 128 | 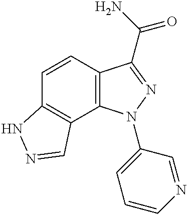 |
| 129 | 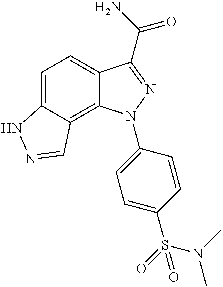 |
| 130 | 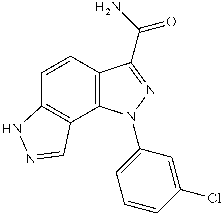 |
| 131 | 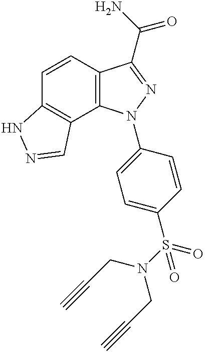 |
| 132 | 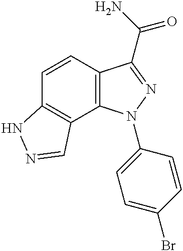 |
| 133 | 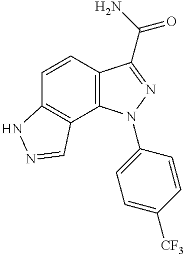 |

In other embodiments, compounds for use as described herein include the compounds set forth below as described in the following U.S. patents and U.S. patent applications:

### U.S. Pat. Nos. 7,064,215 and 7,642,278

The above-named patent describes compounds having the following formula:

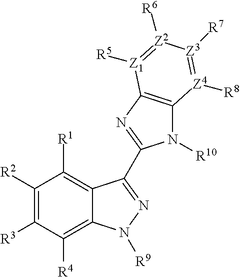

U.S. Pat. Nos. 7,064,215 and 7,642,278 are hereby incorporated by reference in their entirety.

### U.S. Pat. No. 6,897,208

The above-named patent describes compounds having the following formula:

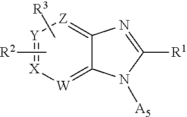

U.S. Pat. No. 6,897,208 is hereby incorporated by reference in its entirety.

### U.S. Publication No. 2006/0014756

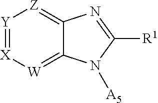

U.S. Publication No. 2006/0014756 is hereby incorporated by reference in its entirety.

In other embodiments, compounds for use as described herein include the compounds set forth below as described in the following published international PCT applications, published foreign patent applications, and published articles:

### PCT International Application No. WO 2001/053268A2

The above-named PCT application describes compounds having the following formula:

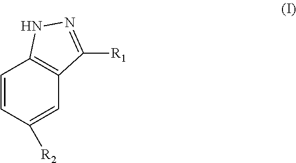

PCT Application No. WO 2001/053268 is hereby incorporated by reference in its entirety.

### PCT International Application No. WO 2003/097610A1

The above-named PCT application describes compounds having the following formula:

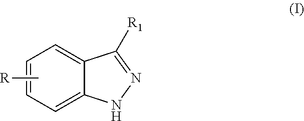

R=halo, (un)substituted alkenyl, alkynyl, (hetero)aryl (attached to position 5 or 6 of the indazole ring); R₁=N:CHNR₂R₃, NHCOR₄, NHCONR₄R₅, NHSO₂R₄, NHCO₂R₄; R₂, R₃=H, alkyl; R₄, R₅=H, alkyl, cycloalkyl, aryl, etc.

PCT Application No. WO 2003/097610 is hereby incorporated by reference in its entirety.

### PCT International Application No. WO 2005/009997A1

The above-named PCT application describes compounds having the following formula:

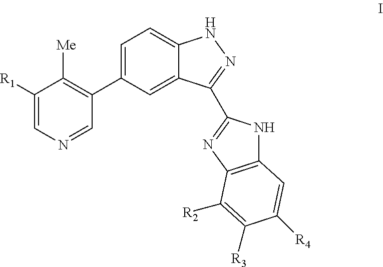

PCT Application No. WO 2005/009997 is hereby incorporated by reference in its entirety.

### PCT International Applications No. WO 2006/054143A1 and WO 2006/054151A1

The above-named PCT application describes a compound having the following formula:

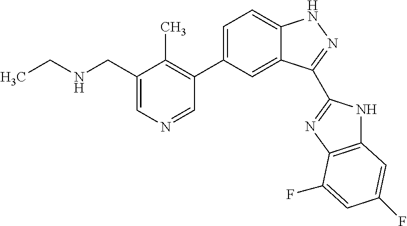

PCT Applications No. WO 2006/054143 and WO 2006/054151 are hereby incorporated by reference in their entirety.

### PCT International Application No. WO 2006/063841A2

The above-named PCT application describes a compound having the following formula:

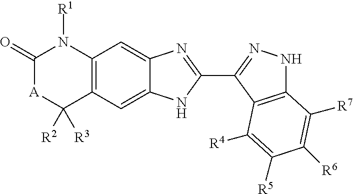

PCT Applications No. WO 2006/063841 is hereby incorporated by reference in its entirety.

### PCT International Application No. WO 2007/107346A1

The above-named PCT application describes compounds having the following formula:

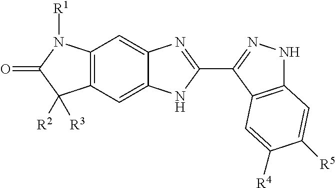

PCT Application No. WO 2007/107346 is hereby incorporated by reference in its entirety.

### PCT International Application No. WO 2008/071451A1

The above-named PCT application describes compounds having the following formula:

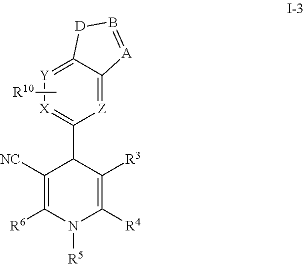

PCT Application No. WO 2008/071451 is hereby incorporated by reference in its entirety.

### PCT International Application No. WO 2003/070706A1

The above-named PCT application describes compounds having the following formula:

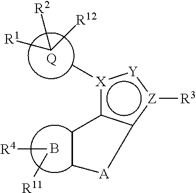

PCT Application No. WO 2003/070706 is hereby incorporated by reference in its entirety.

### PCT International Application No. WO 2003/070236A2

The above-named PCT application describes compounds having the following formula:

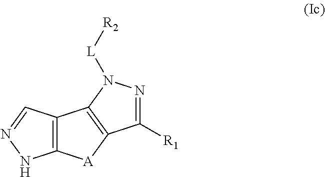

PCT Application No. WO 2003/070236 is hereby incorporated by reference in its entirety.

### “Design and Structure-Activity Relationship of 3-benzimidazol-2-yl-1H-indazoles as Inhibitors of Receptor Tyrosine Kinases”, McBride, Christopher M.; Renhowe, Paul A.; Heise, Carla; Jansen, Johanna M.; Lapointe, Gena; Ma, Sylvia; Pineda, Ramon; Vora, Jayesh; Wiesmann, Marion; Shafer, Cynthia M., Bioorganic & Medicinal Chemistry Letters (2006), 16(13), 3595-3599.

The above-named article describes compounds having the following formula:

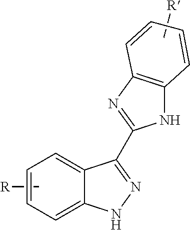

Journal article, Bioorganic & Medicinal Chemistry Letters (2006), 16(13), 3595-3599, is hereby incorporated by reference in its entirety.

### “3D-QSAR CoMFA and CoMSIA Study on Benzodipyrazoles as Cyclin Dependent Kinase 2 Inhibitors”, Dessalew, Nigus; Singh, Sanjeev Kumar, Medicinal Chemistry (2008), 4(4), 313-321

The above-named article describes compounds having the following formula:

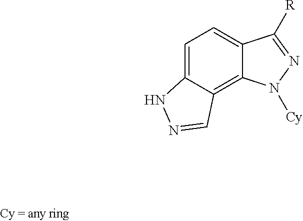

Journal article, Medicinal Chemistry (2008), 4(4), 313-321, is hereby incorporated by reference in its entirety.

### “Structure-Based Approaches to Improve Selectivity: CDK2-GSK3β Binding Site Analysis”, Vulpetti, Anna; Crivori, Patrizia; Cameron, Alexander; Bertrand, Jay; Brasca, Maria Gabriella; D'Alessio, Roberto; Pevarello, Paolo, Journal of Chemical Information and Modeling (2005), 45(5), 1282-1290.

The above-named article describes the following compound:

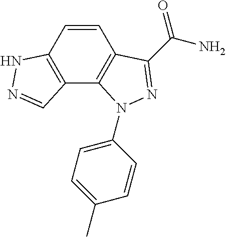

Journal article, Journal of Chemical Information and Modeling (2005), 45(5), 1282-1290, is hereby incorporated by reference in its entirety.

### “Benzodipyrazoles: a New Class of Potent CDK2 Inhibitors”, D'Alessio, Roberto; Bargiotti, Alberto; Metz, Suzanne; Brasca, M. Gabriella; Cameron, Alexander; Ermoli, Antonella; Marsiglio, Aurelio; Polucci, Paolo; Roletto, Fulvia; Tibolla, Marcellino; et al, Bioorganic & Medicinal Chemistry Letters (2005), 15(5), 1315-1319.

The above-named article describes compounds having the following formula:

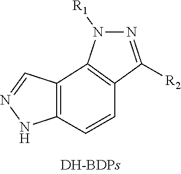

Journal article, Bioorganic & Medicinal Chemistry Letters (2005), 15(5), 1315-1319., is hereby incorporated by reference in its entirety.

Compound Preparation

The starting materials used in preparing the compounds of the invention are known, made by known methods, or are commercially available. It will be apparent to the skilled artisan that methods for preparing precursors and functionality related to the compounds claimed herein are generally described in the literature. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March's Advanced Organic Chemistry: Reactions, Mechanisms, and Structure 6^{th} Ed., John Wiley & Sons (2007), Carey and Sundberg, Advanced Organic Chemistry 5^{th} Ed., Springer (2007), Comprehensive Organic Transformations: A Guide to Functional Group Transformations, 2^{nd} Ed., John Wiley & Sons (1999) (incorporated herein by reference in its entirety) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

The following example schemes are provided for the guidance of the reader, and represent preferred methods for making the compounds exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

¹H nuclear magnetic resonance spectra (NMR) were measured in the indicated solvents on a Bruker NMR spectrometer (Avance™ DRX300, 300 MHz for 1H). Peak positions are expressed in parts per million (ppm) downfield from tetramethylsilane. The peak multiplicities are denoted as follows, s, singlet; d, doublet; t, triplet; m, multiplet.

The following abbreviations have the indicated meanings:

brine=saturated aqueous sodium chloride

Boc₂O=di-tert-butyl dicarbonate

CDCl₃=deuterated chloroform

DCM=dichloromethane

DHP=3,4-dihydro-2H-pyran

DMA=dimethylacetamide

DMF=N,N-dimethylformamide

DMSO-d₆=deuterated dimethylsulfoxide

ESIMS=electron spray mass spectrometry

EtOAc=ethyl acetate

Et₃SiH=triethylsilane

HCl=hydrochloric acid

HOAc=acetic acid

H₂SO₄=sulfuric acid

K₂CO₃=potassium carbonate

KOAc=potassium acetate

KOH=potassium hydroxide

K₃PO₄=potassium phosphate

LAH=lithium aluminum hydride

MeOH=methanol

MgSO₄=magnesium sulfate

Na₂CO₃=sodium carbonate

NaOAc=sodium acetate

NaHCO₃=sodium bicarbonate

NaHSO₃=sodium bisulfite

NaOAc=sodium acetate

Na₂SO₄=sodium sulfate

NH₄OH=ammonium hydroxide

NMR=nuclear magnetic resonance

Pd/C=palladium on carbon

PdCl₂(dppf)₂=1,1′-bis(diphenylphosphino)ferrocene-palladium(II)dichloride

Pd(PPh₃)₂Cl₂=dichloro-bis(triphenylphosphine)palladium (II)

Pd(PPh₃)₄=tetrakis(triphenylphosphine)palladium(0)

PPTS=pyridinium p-toluenesulfonate

rt=room temperature

TFA=trifluoroacetic acid

THF=tetrahydrofuran

THP=tetrahydropyranyl

TLC=thin layer chromatography

The following example schemes are provided for the guidance of the reader, and collectively represent an example method for making the compounds provided herein. Furthermore, other methods for preparing compounds of the invention will be readily apparent to the person of ordinary skill in the art in light of the following reaction schemes and examples. Unless otherwise indicated, all variables are as defined above.

### General Procedures

Compounds of Formula I where R³ is —C(═O)N(R⁹)₂ can be prepared as depicted in Scheme 1.

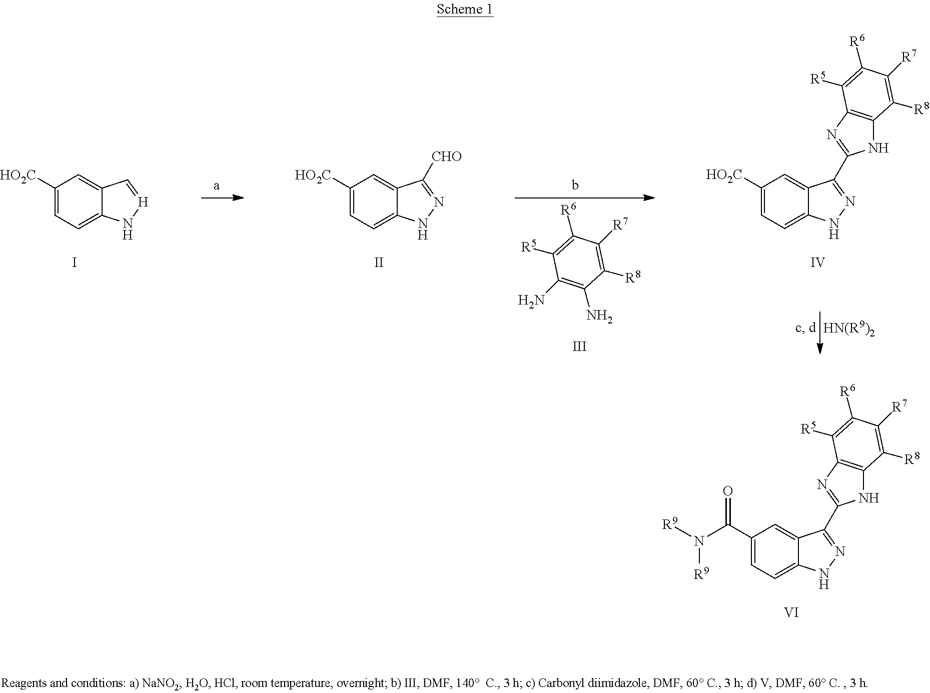

Scheme 1 describes a method for preparation of indazole derivatives (VI) by first reacting 1H-indazole-5-carboxylic acid (I) with acidic aqueous sodium nitrite to form the aldehyde (II). Cyclization with various substituted phenylenediamines (III) yields indazole derivatives (IV). The carboxylic acid (IV) is reacted with various amines (V) to produce the desired indazole derivatives (VI).

Compounds of Formula I where R³ is heterocyclylR¹² can be prepared as depicted in Scheme 2.

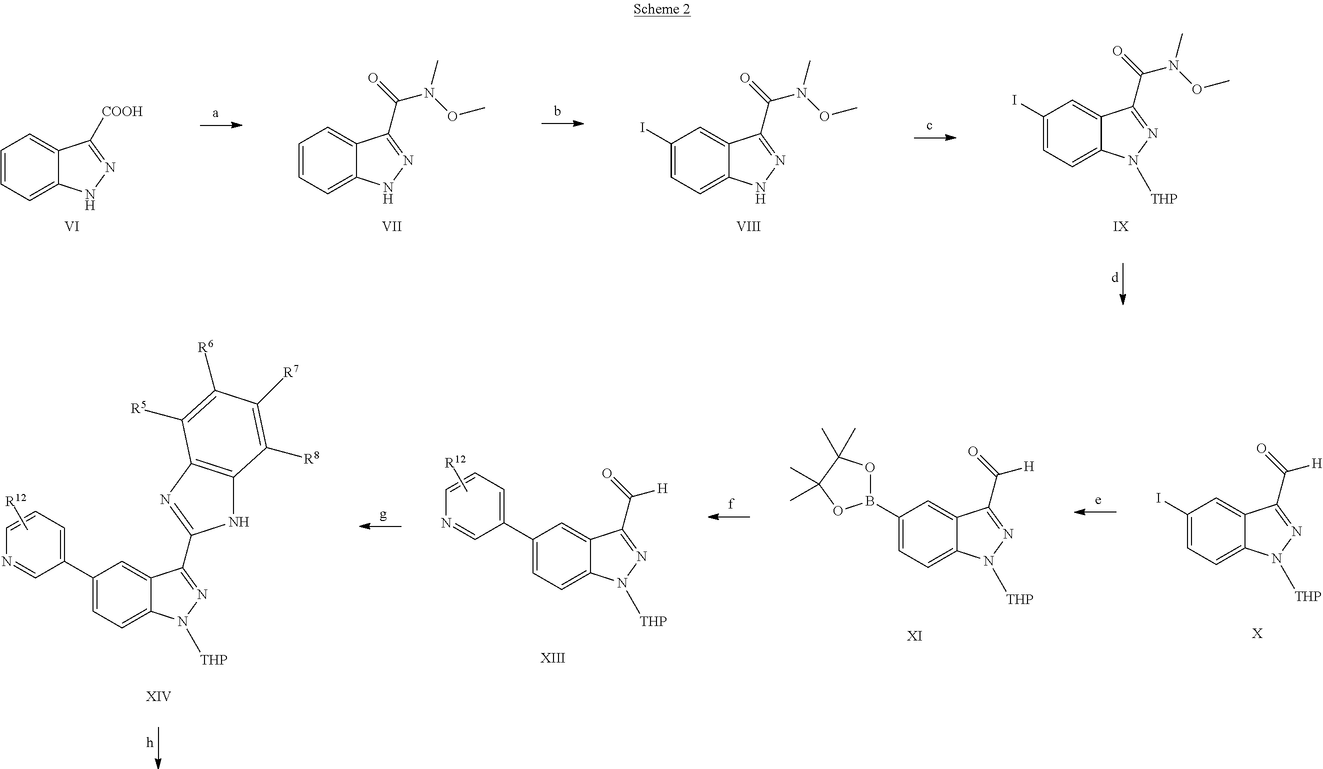

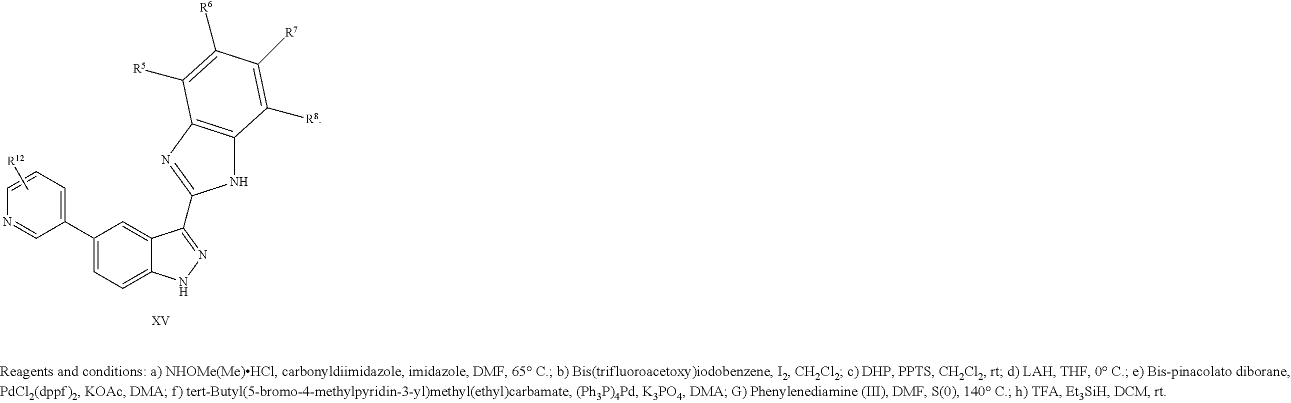

Scheme 2 describes a method for preparation of indazole derivatives (XV) by first reacting 1H-indazole-3-carboxylic acid (VI) with N,O-dimethylhydroxylamine to form the Weinreb amide (VII). Iodozation followed by protection of the 1-nitrogen with THP produce intermediate IX. Reduction of the Weinreb amide gave aldehyde X, which is then treated with an alkyl or aryl boronic acid or ester (e.g., bis-pinacolato diborane) and a suitable Pd catalyst, for example, PdCl₂(dppf) to provide intermediate XI. Intermediate XI is then reacted with a heteroaryl and a suitable Pd catalyst (e.g., (PH₃P)₄Pd) under basic conditions to afford intermediate XIII. XIII is then heated with a Phenylenediamine (III) to provide intermediate XIV. Intermediate XIV is then deprotected to provide the final compound XV. Deprotection conditions are consistent with the specific protecting group employed, for example, acidic conditions for removal of a THP protecting group.

### ILLUSTRATIVE COMPOUND EXAMPLES

Preparation of intermediate (XVII) is depicted below in Scheme 3.

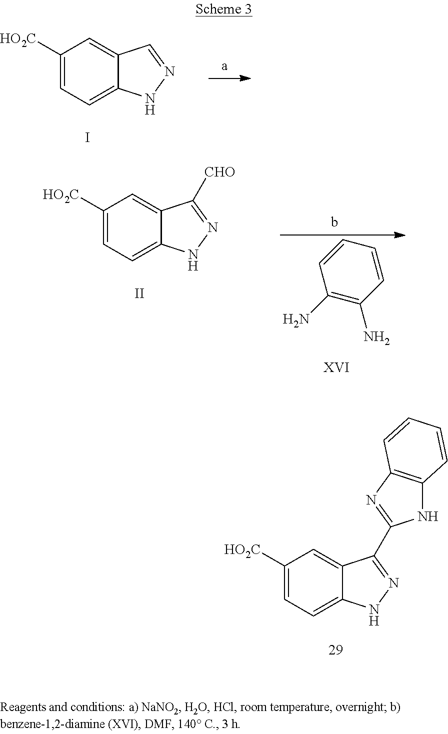

### Step a

NaNO₂ (11.77 g, 170.6 mmol) was added to a suspension of indole-5-carboxylic acid (I) (2.75 g, 17.06 mmol) in water. An aqueous 6 N solution of HCl (21 mL) was added slowly to the solution with vigorously stirring. The solution was further stirred at room temperature for overnight. The solids formed were filtered, washed with water and dried under vacuum at room temperature to get 3-formyl-1H-indazole-5-carboxylic acid (II) as a red solid (1.80 g, 105.6 mmol, 55% yield). ¹H NMR (DMSO-d₆) δ ppm 7.79 (d, J=8.85 Hz, 1H), 8.05 (dd, J=8.85, 1.32 Hz, 1H), 8.78 (s, 1H), 10.23 (s, 1H), 14.40 (br s, 1H); ESIMS found C₉H₆N₂O₃ m/z 191 (M+H).

### Step b

A solution of 3-formyl-1H-indazole-5-carboxylic acid (II) (1.67 g, 8.78 mmol), benzene-1,2-diamine (XVI) (1.04 g, 9.66 mmol) and sulfur (0.31 g, 9.66 mmol) in DMF was heated at 140° C. for 3 h under nitrogen. The solution was cooled and the excess DMF was evaporated under vacuum. Water was added to the residue and sonicated briefly to suspend the solids. The solids were filtered, washed with cold water and air dried at room temperature. The solids were boiled in methanol and the insoluble solids were filtered, washed with cold methanol and dried at room temperature under vacuum to get 3-(1H-benzimidazol-2-yl)-1H-indazole-5-carboxylic acid (29) as a off white solid (1.3 g, 4.67 mmol, 53% yield). ¹H NMR (DMSO-d₆) δ ppm 7.15-7.32 (m, 2H), 7.49-7.60 (m, 1H), 7.73 (d, J=8.85 Hz, 1H), 7.77-7.87 (m, 1H), 8.04 (dd, J=8.85, 1.32 Hz, 1H), 9.23 (s, 1H), 13.09 (s, 1H), 13.91 (br s, 1H); ESIMS found C₁₅H₁₀N₄O₂ m/z 279 (M+H).

Preparation of intermediate (XX) is depicted below in Scheme 4.

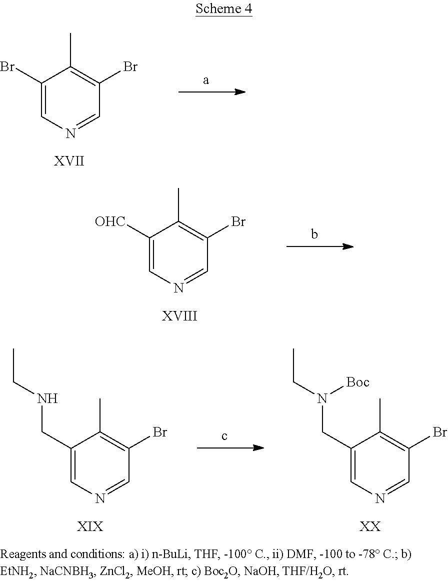

### Step a

n-Butyllithium (2.5 M in hexanes, 6.2 mL, 15.4 mmol) was added dropwise to a solution of 3,5-dibromo-4-methyl-pyridine (XVII) (3.8 g, 15.1 mmol) in dry THF stirred at −100° C. under argon. The reaction was further stirred for 5 minutes at the same temperature before adding dry DMF (1.8 mL, 23.2 mmol). The solution was further stirred at −100° C. for 20 minutes and then at −78° C. for 1 hour. The reaction was quenched with saturated NH₄Cl solution and extracted with ether. The combined organic phases were washed with brine, dried over Na₂SO₄, and concentrated in vacuo. Purification of crude product by flash chromatography gave 5-bromo-4-methylnicotinaldehyde (XVIII). ¹H NMR (CDCl₃, 400 MHz) δ ppm 2.80 (s, 3H), 8.84 (m, 2H), 10.20 (s, 1H).

### Step b

A solution of ethylamine in methanol (2.0 M, 90 mL, 180 mmol) was added dropwise over 30 minutes to a stirred solution of 5-bromo-4-methylnicotinaldehyde (XVIII) (6.74 g, 33.7 mmol) in methanol under nitrogen. The solution was further stirred 30 minutes at room temperature. In a separate flask, sodium cyanoborohydride (2.33 g, 37.1 mmol) was dissolved in methanol and was combined with anhydrous zinc chloride (2.53 g, 18.5 mmol). The solution was further stirred at room temperature for 20 minutes. This solution was added slowly to the above ethylamine/aldehyde solution. The reaction solution was acidified to pH 4 with 2.0 M HCl in methanol (120 mL) and then stirred at room temperature for 18 hours. Solvent was removed by rotary evaporation and the residue was partitioned between ethyl acetate and 10% aqueous sodium carbonate. The organic extracts were dried over MgSO₄ and concentrated in vacuo to provide N-((5-bromo-4-methylpyridin-3-yl)methyl)ethanamine (XIX). XIX was used without purification for step c.

### Step c

Di-tert-butyl dicarbonate (10.43 g, 47.8 mmol) was added to a solution of N-((5-bromo-4-methylpyridin-3-yl)methyl)ethanamine (XIX) (7.36 g, 32.1 mmol) in THF (400 mL), followed by an aqueous NaOH solution (1.0 M, 101 mL). The biphasic solution was stirred vigorously for 20 hours at room temperature. The solution was then partitioned between water and ethyl acetate. The organic phase was dried over MgSO₄, filtered, and concentrated. The residue was purified by flash chromatography to provide tert-butyl (5-bromo-4-methylpyridin-3-yl)methyl(ethyl)carbamate (XX). ¹H NMR (CDCl₃, 400 MHz) δ ppm 1.10 (t, 3H), 1.48 (s, 9H), 2.40 (s, 3H), 3.20 (m, 2H), 4.53 (s, 2H), 8.26 (m, 1H), 8.63 (s, 1H).

Preparation of intermediate (XXIV) is depicted below in Scheme 5.

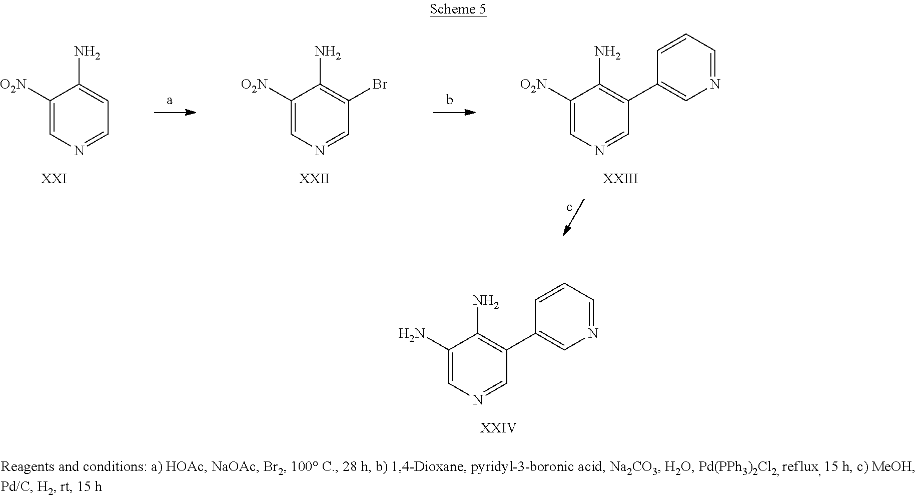

### Step a

A mixture of 3-nitropyridin-4-amine (XXI) (10 g, 71.94 mmol) and acetic acid (120 ml) was added to a sealed tube followed by addition of NaOAc (29.50 g, 93.52 mmol) and dropwise addition of bromine (4.7 ml 359.7 mmol) under stirring. The sealed tube was heated at 100° C. for 28 h until TLC showed consumption of starting material. The reaction mixture was concentrated to obtain a solid which was dissolved in water, basified with NaHCO₃ and extracted with ethyl acetate. The combined organic extracts were dried and concentrated to produce 3-bromo-5-nitropyridin-4-amine (XXII) as a yellow solid (12 g, 55 mmol, 77% yield). ¹H NMR (DMSO-d₆) δ ppm 9.19 (s, 1H), 8.58 (s, 1H); ESIMS found for C₅H₄BrN₃O₂ m/z 217, 219 (M+, M+2).

### Step b

A solution of 3-bromo-5-nitropyridin-4-amine (XXII) (6 g, 26 mmol), pyridin-3-ylboronic acid (3.54 g, 29 mmol), 1 N Na₂CO₃ solution (78 ml) and 1,4-dioxane (150 mL) was degassed with argon thrice. Pd(PPh₃)₂Cl₂ (927 mg, 5 mmol %) was added to the reaction and the solution was refluxed for 15 h until TLC showed the reaction was complete. The reaction was passed through a pad of Celite and then concentrated under reduced pressure. The reaction mixture was concentrated and the residue was taken up in ethyl acetate. The organic extract was washed with water, dried and concentrated under vacuum. The crude product was purified on a silica gel column (100% EtOAc→>2:98 MeOH:DCM) to give 5-nitro-3,3′-bipyridin-4-amine (XXIII) as a yellow solid (5 g, 23.1 mmol, 87% yield). ¹H NMR (CDCl₃, 400 MHz), δ ppm 9.31 (s, 1H), 8.80-8.79 (m, 1H), 8.70 (s, 1H), 8.23 (s, 1H), 7.80-7.73 (m, 1H), 7.52-7.48 (m, 1H). ESIMS found C₁₀H₈N₄O₂ m/z 216.95 (M+H).

### Step c

To a solution of 5-nitro-3,3′-bipyridin-4-amine (XXIII) (5 g, 23 mmol) in MeOH (20 mL) was added 10% Pd/C. The solution was purged with hydrogen and stirred at r.t. under hydrogen for 15 h. The suspension was filtered through Celite and the concentrated under vacuum to produce 3,3′-bipyridine-4,5-diamine (XXIV) as off white solid (3.3 g, 17.7 mmol, 76% yield). ¹H NMR (DMSO-d₆, 400 MHz): δ 8.63-8.53 (m, 1H), 7.90-7.83 (m, 1H), 7.75 (s, 1H), 7.58 (s, 1H), 7.48-7.43 (m, 2H), 6.13 (bs, 2H), 5.31 (bs, 2H). ESIMS found C₁₀H₁₀N₄ m/z 187.10 (M+H).

Preparation of intermediate (XXVII) is depicted below in Scheme 6.

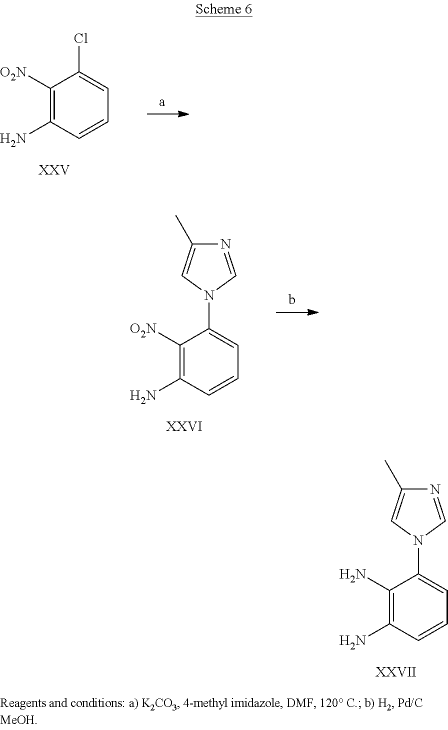

### Step a

A solution of 3-chloro-2-nitro-aniline (XXV) (1.0 g, 5.8 mmol), potassium carbonate (2.4 g, 17.4 mmol), and 4-methylimidazole in dry DMF was heated overnight at 120° C. under nitrogen. The reaction was cooled and the solvent was evaporated in vacuo. The residue was suspended in a saturated NaHCO₃ solution and extracted with CH₂Cl₂. The combined organic phases were dried over MgSO₄ and concentrated in vacuo. The crude product was purified by flash chromatography to provide 3-(4-methyl-imidazol-1-yl)-2-nitro-phenylamine (XXVI). ¹H NMR (CDCl₃, 400 MHz) δ ppm 2.19 (s, 3H), 6.53 (m, 1H), 6.79 (m, 1H), 6.93 (m, 1H), 7.32 (m, 1H), 7.60 (m, 1H).

### Step b

To a solution of 3-(4-methyl-imidazol-1-yl)-2-nitro-phenylamine (XXVI) in methanol was added with 5% Pd/C. The combination was stirred under a hydrogen filled balloon at 40° C. for 6 hours. The solution was then filtered through a pad of Celite. The filtrate was concentrated in vacuo to get 3-(4-methyl-imidazol-1-yl)-benzene-1,2-diamine (XXVII). ¹H NMR (CDCl₃, 400 MHz) δ ppm 2.17 (s, 3H), 6.54 (m, 1H), 6.80 (m, 1H), 6.97 (m, 1H), 7.28 (m, 1H), 7.56 (m, 1H).

### Example 1

Preparation of 3-(1H-benzo[d]imidazol-2-yl)-N-(pentan-3-yl)-1H-indazole-5-carboxamide (1) is depicted below in Scheme 7.

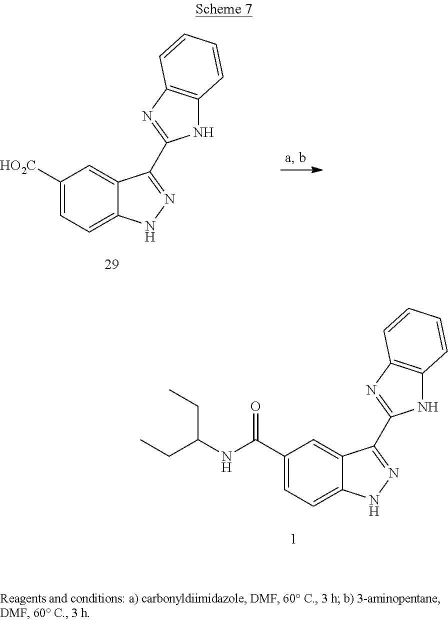

### Step a-b

Carbonyldiimidazole (0.525 g, 3.24 mmol) was added to a solution of 3-(1H-benzimidazol-2-yl)-1H-indazole-5-carboxylic acid (29) (0.82 g, 2.95 mmol) in DMF while stirring at room temperature under nitrogen. The solution was heated at 60° C. for 3 h before cooling to room temperature. 3-Aminopentane (0.282 g, 3.24 mmol) was added to the solution and again heated at 60° C. for another 3 h. The solution was cooled and concentrated under vacuum. The residue was dissolved in dichloromethane, washed successively with saturated NaHCO₃ solution, water and brine, dried over MgSO₄, filtered and concentrated. The crude product was purified by flash chromatography to get 3-(1H-benzo[d]imidazol-2-yl)-N-(pentan-3-yl)-1H-indazole-5-carboxamide (1) as a off white solid (0.578 g, 1.66 mmol, 46% yield). ¹H NMR (DMSO-d₆) δ ppm 0.91 (t, J=7.35 Hz, 6H), 1.47-1.64 (m, 4H), 3.80-3.92 (m, 1H), 7.23 (dd, J=6.03, 3.20 Hz, 2H), 7.68 (d, J=8.85 Hz, 2H), 7.95 (dd, J=8.76, 1.41 Hz, 1H), 8.19 (d, J=8.76 Hz, 1H), 9.00 (s, 1H); ESIMS found C₂₀H₂₁N₅O m/z 348 (M+H).

The following compounds was prepared in accordance with the procedure described in the above Example 1.

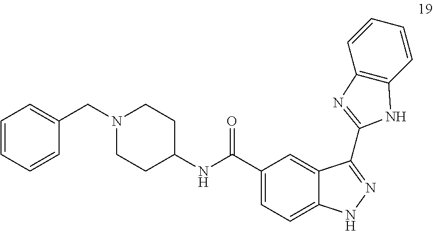

### 3-(1H-benzo[d]imidazol-2-yl)-N-(1-benzylpiperidin-4-yl)-1H-indazole-5-carboxamide 19

Off white solid (0.307 g, 0.68 mmol, 94% yield). ¹H NMR (DMSO-d₆) δ ppm 1.65 (m, 2H), 1.85 (m, 2H), 2.06 (m, 2H), 2.87 (m, 2H), 3.49 (s, 2H), 7.25-7.33 (m, 8H), 7.67 (d, J=8.67 Hz, 1H), 7.92 (d, J=8.67 Hz, 1H), 8.42 (d, J=7.91 Hz, 1H), 8.98 (s, 1H); ESIMS found C₂₇H₂₆N₆O m/z 451 (M+H).

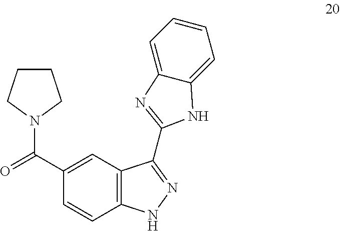

### (3-(1H-benzo[d]imidazol-2-yl)-1H-indazol-5-yl)(pyrrolidin-1-yl)methanone 20

Off white solid (67% yield). ¹H NMR (DMSO-d₆) δ ppm 1.81-1.95 (m, 4H), 3.44-3.59 (m, 4H), 7.23 (dd, J=5.93, 3.11 Hz, 2H), 7.52-7.74 (m, 4H), 8.70 (s, 1H); ESIMS found C₁₉H₁₇N₅O m/z 332 (M+H).

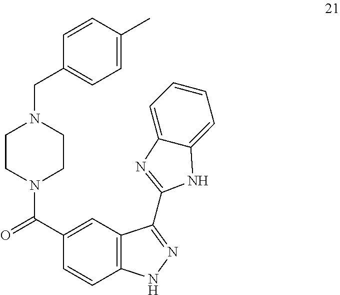

### (3-(1H-benzo[d]imidazol-2-yl)-1H-indazol-5-yl)(4-(4-methylbenzyl)piperazin-1-yl)methanone 21

Off white solid (14% yield). ¹H NMR (DMSO-d₆) δ ppm 2.28 (m, 4H), 2.42 (m, 4H), 3.49 (s, 2H), 7.01-7.163 (m, 3H), 7.16-7.30 (m, 4H), 7.47 (d, J=8.67 Hz, 1H), 7.52 (d, J=6.78 Hz, 1H), 7.68 (m, 1H), 7.75 (d, J=8.10 Hz, 1H), 8.58 (s, 1H); ESIMS found C₂₇H₂₆N₆O m/z 451 (M+H).

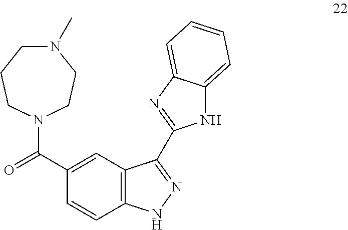

### (3-(1H-benzo[d]imidazol-2-yl)-1H-indazol-5-yl)(4-methyl-1,4-diazepan-1-yl)methanone 22

Off white solid (81% yield). ¹H NMR (DMSO-d₆) δ ppm 2.28 (m, 4H), 2.68 (m, 4H), 7.22 (dd, J=5.93, 3.11 Hz, 2H), 7.47 (d, J=8.48 Hz, 1H), 7.63 (m, 1H), 7.69 (d, J=8.67 Hz, 1H), 8.56 (s, 1H); ESIMS found C₂₁H₂₂N₆O m/z 375 (M+H).

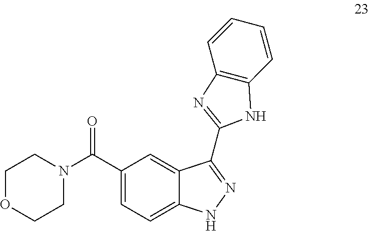

### (3-(1H-benzo[d]imidazol-2-yl)-1H-indazol-5-yl)(morpholino) methanone 23

White solid (44% yield). ¹H NMR (DMSO-d₆) δ ppm 3.63 (m, 4H), 7.23 (m, 4H), 7.50 (d, J=8.67 Hz, 1H), 7.61 (m, 1H), 8.60 (s, 1H); ESIMS found C₁₉H₁₇N₅O₂ m/z 348 (M+H).

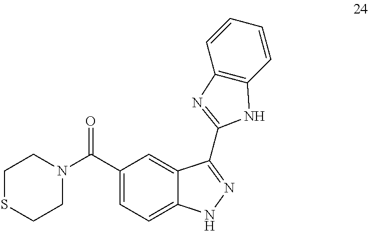

### (3-(1H-benzo[d]imidazol-2-yl)-1H-indazol-5-yl)(thiomorpholino)methanone 24

White solid (42% yield). ¹H NMR (DMSO-d₆) δ ppm 2.73 (m, 4H), 7.22 (m, 4H), 7.49 (d, J=8.48 Hz, 1H), 7.70 (d, J=8.67 Hz, 1H), 8.57 (s, 1H); ESIMS found C₁₉H₁₇N₅OS m/z 364 (M+H).

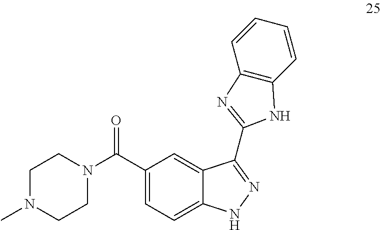

### [3-(1H-benzimidazol-2-yl)-1H-indazole-5-yl]-(4-methyl-piperazin-1-yl)-methanone 25

White solid (43% yield). ¹H NMR (DMSO-d₆) δ ppm 2.21 (s, 3H), 2.35 (m, 4H), 7.23 (dd, J=5.93, 3.11 Hz, 4H), 7.48 (dd, J=8.57, 1.41 Hz, 1H), 7.70 (d, J=8.67 Hz, 1H), 8.58 (s, 1H); ESIMS found C₂₀H₂₀N₆O m/z 361 (M+H).

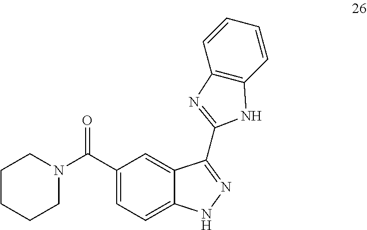

### (3-(1H-benzo[d]imidazol-2-yl)-1H-indazol-5-yl)(piperidin-1-yl)methanone 26

White solid (87% yield). ¹H NMR (DMSO-d₆) δ ppm 1.64 (m, 6H), 7.22 (m, 2H), 7.38 (m, 2H), 7.63 (m, 2H), 8.57 (s, 1H); ESIMS found C₂₀H₁₉N₅O m/z 346 (M+H).

### Example 2

Preparation of 3-(1H-benzo[d]imidazol-2-yl)-1H-indazole-5-carbonitrile (27) and 3-(1H-benzo[d]imidazol-2-yl)-1H-indazole-5-carboxamide (28) is depicted below in Scheme 8.

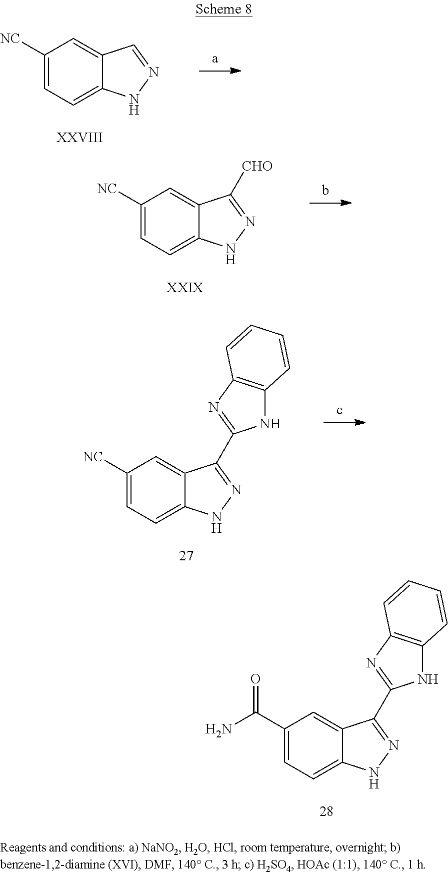

### Step a

Same procedure as in Scheme 3, Step a. 3-formyl-1H-indazole-5-carbonitrile (XXIX) isolated as a orange solid (79% yield). ¹H NMR (DMSO-d₆) δ ppm 7.83-7.93 (m, 2H), 8.59 (s, 1H), 10.23 (s, 1H).

### Step b

A solution of 3-formyl-1H-indazole-5-carbonitrile (XXIX) (2 g, 11.6 mmol), benzene-1,2-diamine (XVI) and sulfur in DMF was heated 3 h at 140° C. The solution was cooled and concentrated under vacuum. The residue was treated with water, sonicated briefly to disperse the solids and filtered. The solids were washed with cold water and dried at room temperature. The crude product was purified by flash chromatography eluting with 0-1% MeOH in CH₂Cl₂ gradient to get 3-(1H-benzo[c/]imidazol-2-yl)-1H-indazole-5-carbonitrile (27) as off white solid (0.72 g, 2.77 mmol, 24% yield). ¹H NMR (DMSO-d₆) δ ppm 7.25 (dd, J=5.93, 3.11 Hz, 2H), 7.78 (m, 2H), 7.84 (m, 2H), 8.96 (s, 1H); ESIMS found C₁₅H₉N₅ m/z 260 (M+H).

### Step c

Sulfuric acid (4 mL) was added carefully to a mixture of 3-(1H-benzo[d]imidazol-2-yl)-1H-indazole-5-carbonitrile (27) (0.178 g, 0.69 mmol) and glacial acetic acid (4 mL) at room temperature. The solution was heated at 140° C. for 1 h. The solution was then poured into ice and basified to pH 9.0 by adding concentrated ammonium hydroxide solution. The solution was stirred for 30 min and the solids formed were filtered, washed by cold water and dried under vacuum at room temperature to get 3-(1H-benzo[d]imidazol-2-yl)-1H-indazole-5-carboxamide (28) as a off white solid (0.172 g, 0.62 mmol, 90% yield). ¹H NMR (DMSO-d₆) δ ppm 7.21 (m, 2H), 7.48 (m, 1H), 7.67 (d, J=8.85 Hz, 1H), 7.74 (m, 1H), 7.97 (dd, J=8.76, 1.41 Hz, 1H), 9.06 (s, 1H); ESIMS found C₁₅H₁₁N₅O m/z 278 (M+H).

### Example 3

Preparation of 3-(1H-benzo[d]imidazol-2-yl)-N,N-dimethyl-1H-indazole-5-carboxamide (30) is depicted below in Scheme 9.

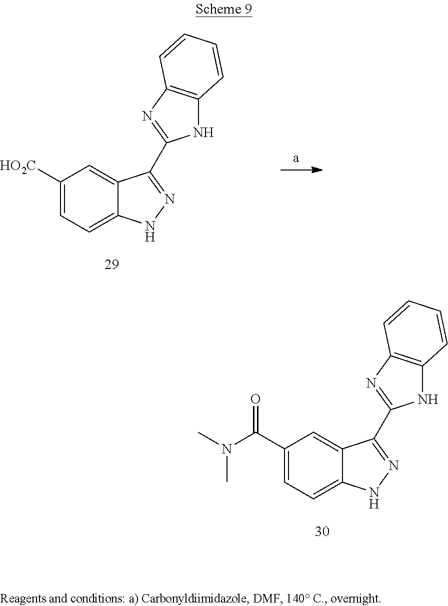

### Step a

Carbonyldiimidazole (0.128 g, 0.79 mmol) was added to a solution of 3-(1H-benzo[d]imidazol-2-yl)-1H-indazole-5-carboxylic acid (29) (0.2 g, 0.72 mmol) in DMF at room temperature and heated at 80° C. for 2 h before raising the temperature to 140° C. The solution was heated overnight at 140° C. The solution was cooled and concentrated under vacuum. The residue was treated with water, sonicated briefly and the solids which formed were filtered. The solids were washed with cold water, dried at room temperature, and purified by flash chromatography eluting with 1-5% MeOH in CH₂Cl₂ gradient to get 3-(1H-benzo[d]imidazol-2-yl)-N,N-dimethyl-1H-indazole-5-carboxamide (30) as a white solid (53 mg, 0.17 mmol, 24% yield). ¹H NMR (DMSO-d₆) δ ppm 3.02 (s, 6H), 7.22 (m, 3H), 7.50 (dd, J=8.57, 1.41 Hz, 1H), 7.65 (m, 2H), 8.57 (s, 1H); ESIMS found C₁₇H₁₅N₅O m/z 306 (M+H).

### Example 4

Preparation of N-((5-(3-(1H-benzo[d]imidazol-2-yl)-1H-indazol-5-yl)-4-methylpyridin-3-yl)methyl)ethanamine (31) is depicted below in Scheme 10.

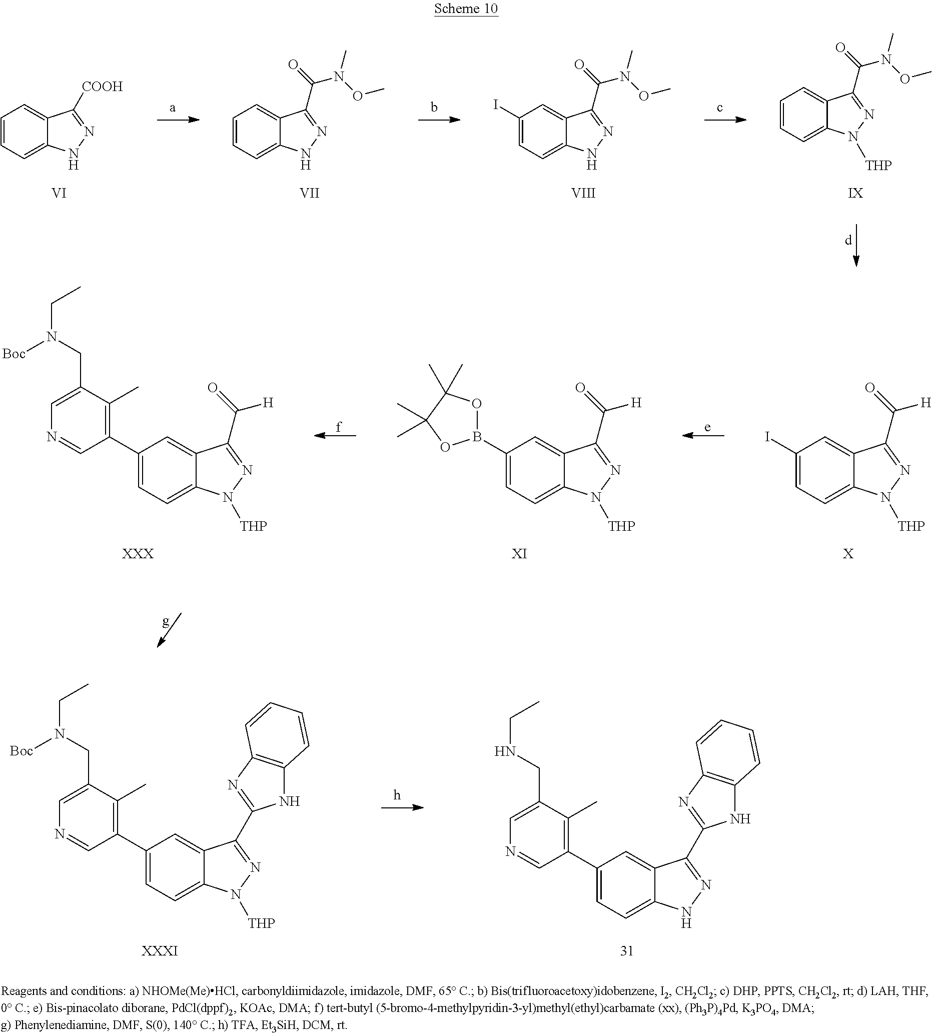

### Step a

1H-indazole-3-carboxylic acid (VI) (100 g, 617 mmol) in DMF was treated with carbonyldiimidazole (110 g, 678 mmol) at 25° C. until the evolution of gas ceased (ca. 15 minutes). The reaction was heated to 60-65° C. for two hours and then allowed to cool to 25° C. N,O-Dimethylhydroxylamine-HCl (66.2 g, 678 mmol) was added as a solid and the mixture was heated to 65° C. for 3 hours. The reaction was concentrated to a paste and taken up in CH₂Cl₂, and washed subsequently with water and 2N HCl. The product was visibly coming out of solution. The solid was filtered and rinsed separately with ethyl acetate. The ethyl acetate and CH₂Cl₂ layers were separately washed with sodium bicarbonate followed by brine, dried over MgSO₄ and concentrated. The resulting solids were combined, triturated with 1:1 mixture of CH₂Cl₂-ether, filtered, and dried to produce N-methoxy-N-methyl-1H-indazole-3-carboxamide (VII) as a white solid (79% yield). ¹H NMR (DMSO-d₆) δ ppm 3.46 (s, 3H), 3.69-3.85 (m, 3H), 7.13-7.31 (m, 1H), 7.41 (t, J=7.25 Hz, 1H), 7.56-7.65 (m, 1H), 7.93-8.08 (m, 1H); ESIMS found for C₁₀H₁₁N₃O₂ m/z 206 (M+H).

### Step b

To the N-methoxy-N-methyl-1H-indazole-3-carboxamide (VII) (29 g, 97.4 mmol) in 1 L CH₂Cl₂ was added bis(trifluoroacetoxy)iodobenzene (46 g, 107 mmol) followed by portionwise addition of iodine (14.84 g, 58.5 mmol) at 25° C. After 1 hour, 600 mL of saturated Na₂HSO₃ was added and a solid began to precipitate which was filtered and rinsed with excess CH₂Cl₂. The filtrate was washed with brine, dried over MgSO₄, concentrated and the remaining solid was triturated with a minimal amount of CH₂Cl₂. The combined solids were dried under vacuum over KOH to produce 5-iodo-N-methoxy-N-methyl-1H-indazole-3-carboxamide (VIII) as a white solid (72% yield). ¹H NMR (DMSO-d₆) δ ppm 3.45 (s, 4H), 3.77 (s, 4H), 7.45-7.54 (m, 1H), 7.66 (dd, J=8.81, 1.51 Hz, 1H), 8.40 (d, J=1.01 Hz, 1H); ESIMS found for C₁₀H₁₀IN₃O₂ m/z 331 (M+H).

### Step c

A mixture of 5-iodo-N-methoxy-N-methyl-1H-indazole-3-carboxamide (VIII) (16.5 g, 50 mmol), 3,4-dihydro-2H-pyran (10.3 mL, 113 mmol) and PPTS (0.12 g, 0.6 mmol) in CH₂Cl₂ was heated to reflux for 5 hours. The solution was poured into a saturated NaHCO₃ solution, the layers were separated, and the aqueous layer was extracted with CH₂Cl₂. The combined organic layers were washed with 5% aqueous citric acid and brine, dried over MgSO₄, and concentrated. The crude product was purified by flash chromatography to provide 5-iodo-N-methoxy-N-methyl-1-(tetrahydro-2H-pyran-2-yl)-1H-indazole-3-carboxamide (IX) as a white viscous oil (92% yield). ¹H NMR (DMSO-d₆) δ ppm 1.28-1.84 (m, 6H), 3.43 (s, 3H), 3.60-4.04 (s, 5H), 5.86-6.08 (m, 1H), 7.45-7.87 (m, 2H), 8.39 (s, 1H); ESIMS found for C₁₅H₁₈IN₃O₃ m/z 416 (M+H).

### Step d

Lithium aluminum hydride (1.2 equiv.) was added portionwise to a cooled (0° C.) solution of 5-iodo-N-methoxy-N-methyl-1-(tetrahydro-2H-pyran-2-yl)-1H-indazole-3-carboxamide (IX) (1.0 equiv.) in THF. Stirring was continued at 0° C. until the reaction was completed, approximately 30 minutes. The reaction was quenched by the slow addition of ethyl acetate at 0° C., and the whole mixture was poured into 0.4 N NaHSO₄. The organic layer was washed with brine, dried over MgSO₄, concentrated, and purified by flash chromatography to get 5-iodo-1-(tetrahydro-2H-pyran-2-yl)-1H-indazole-3-carbaldehyde (X) as a white solid (0.90 g, 3.15 mmol, 72% yield). ¹H NMR (DMSO-d₆) δ ppm 1.50-1.71 (m, 2H), 1.71-1.87 (m, 1H), 1.97-2.15 (m, 2H), 2.31-2.42 (m, 1H), 3.66-3.99 (m, 2H), 5.96-6.17 (m, 1H), 7.78 (d, J=6 Hz, 1H), 7.84 (d, J=6 Hz, 1H), 8.50 (s, 1H), 10.13 (s, 1H); ESIMS found for C₁₃H₁₃IN₂O₂ m/z 357 (M+H).

### Step e-f

A solution of 5-iodo-1-(tetrahydro-2H-pyran-2-yl)-1H-indazole-3-carbaldehyde (X) (0.178 g, 0.5 mmol), bis(pinacolato)diboron (0.152 g, 0.6 mmol) and potassium acetate (0.147 g, 1.5 mmol) in DMF was purged with nitrogen. PdCl₂(dppf)₂ (25 mg) was added to the reaction and purged again with nitrogen. The solution was heated at 80° C. for 2 h. Once TLC showed the disappearance of (X), the solution was cooled to room temperature. To this solution was added K₃PO₄ (0.159 g, 0.75 mmol), tert-butyl (5-bromo-4-methylpyridin-3-yl)methyl(ethyl)carbamate (XX), (0.197 g, 0.6 mmol), Pd(PPh₃)₄ (17 mg, 0.015 mmol) and water (0.5 mL). The solution was purged with nitrogen and heated at 90° C. for 3 h. The reaction was passed through a pad of Celite and then concentrated under reduced pressure. The residue was dissolved in DCM and washed with water and brine, dried over MgSO₄ and then evaporated under vacuum. The crude tert-butyl ethyl((5-(3-formyl-1-(tetrahydro-2H-pyran-2-yl)-1H-indazol-5-yl)-4-methylpyridin-3-yl)methyl)carbamate (XXX) was used without further purification for step g.

### Step g

A solution of tert-butyl ethyl((5-(3-formyl-1-(tetrahydro-2H-pyran-2-yl)-1H-indazol-5-yl)-4-methylpyridin-3-yl)methyl)carbamate (XXX) (0.5 mmol), sulfur (21 mg, 0.66 mmol) and benzene-1,2-diamine (XVI) (71 mg, 0.66 mmol) was heated at 140° C. for 3 h. The solution was cooled and concentrated under vacuum. The residue was treated with water, sonicated briefly and the solids were filtered. The solids were washed with cold water, dried at room temperature and purified by flash chromatography eluting with 0-0.5% MeOH in CH₂Cl₂ gradient to get tert-butyl (5-(3-(1H-benzo[c/]imidazol-2-yl)-1-(tetrahydro-2H-pyran-2-yl)-1H-indazol-5-yl)-4-methylpyridin-3-yl)methyl(ethyl)carbamate (XXXI) as a white solid (72 mg, 0.13 mmol, 25% yield). ¹H NMR (DMSO-d₆) δ ppm 0.92 (m, 3H), 1.42 (s, 9H), 1.67 (m, 2H), 1.80 (m, 2H), 2.20 (s, 3H), 2.73 (m, 2H), 3.23 (m, 2H), 3.84 (m, 2H), 4.54 (s, 2H), 6.06 (m, 1H), 7.19 (m, 2H), 7.51 (m, 2H), 7.72 (m, 1H), 7.96 (m, 1H), 8.33 (s, 1H), 8.41 (s, 1H), 8.46 (s, 1H), 13.01 (s, 1H); ESIMS found for C₃₃H₃₈N₆O₃ m/z 567 (M+H).

### Step h

Trifluoro acetic acid (0.39 mL) was added to a solution of tert-butyl (5-(3-(1H-benzo[d]imidazol-5-yl)-4-methylpyridin-3-yl)methyl(ethyl)carbamate methylpyridin-3-yl)methyl(ethyl)carbamate (XXXI) (60 mg, 0.105 mmol) and triethylsilane (31 mg, 0.26 mmol) in dichloromethane. The solution was stirred for 3 h at room temperature. The solution was concentrated under vacuum and the residue was treated with water and basified by using aqueous 2N NH₄OH solution. The solids formed were filtered, washed by cold water and dried under vacuum at room temperature to get N-((5-(3-(1H-benzo[d]imidazol-2-yl)-1H-indazol-5-yl)-4-methylpyridin-3-yl)methyl)ethanamine (31) as a white solid (32 mg, 0.08 mmol, 80% yield). ¹H NMR (DMSO-d₆) δ ppm 1.09 (t, J=7.06 Hz, 3H), 2.27 (s, 3H), 2.63 (m, 2H), 3.79 (s, 2H), 7.12-7.20 (m, 2H), 7.45 (d, J=8.57 Hz, 1H), 7.52 (d, J=7.35 Hz, 1H), 7.70 (d, J=7.54 Hz, 1H), 7.76 (d, J=8.67 Hz, 1H), 8.36 (s, 1H), 8.43 (s, 1H), 8.48 (s, 1H), ESIMS found for C₂₃H₂₂N₆ m/z 383 (M+H).

The following compounds was prepared in accordance with the procedure described in the above Example 4. Some of the substituted benzene-1,2-diamine intermediates were prepared in accordance with the procedures described in Schemes 5 and 6.

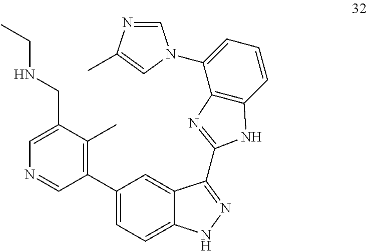

### N-((4-methyl-5-(3-(4-(4-methyl-1H-imidazol-1-yl)-1H-benzo[d]imidazol-2-yl)-1H-indazol-5-yl)pyridin-3-yl)methyl)ethanamine 32

¹H NMR (DMSO-d₆) δ ppm 1.10 (t, 3H), 2.18 (s, 3H), 2.39 (s, 3H), 2.63 (m, 2H), 3.82 (s, 2H), 7.30 (m, 1H), 7.40 (m, 1H), 7.52 (m, 1H), 7.82 (m, 1H), 7.93 (s, 1H), 8.38 (s, 1H), 8.45 (s, 1H), 8.74 (s, 1H), ESIMS found for C₂₇H₂₆N₈ m/z 463 (M+H).

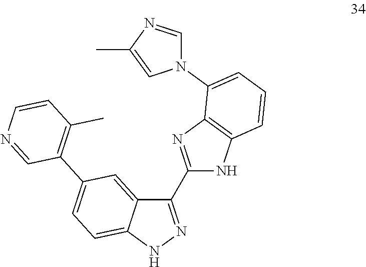

### 3-(4-(4-methyl-1H-imidazol-1-yl)-1H-benzo[d]imidazol-2-yl)-5-(4-methylpyridin-3-yl)-1H-indazole 34

¹H NMR (DMSO-d₆) δ ppm 2.17 (s, 3H), 2.38 (s, 3H), 7.30 (m, 1H), 7.20-7.60 (m, 4H), 7.80-7.95 (m, 2H), 8.40-8.60 (m, 2H), 8.75 (s, 1H), ESIMS found for C₂₄H₁₉N₇ m/z 406 (M+H).

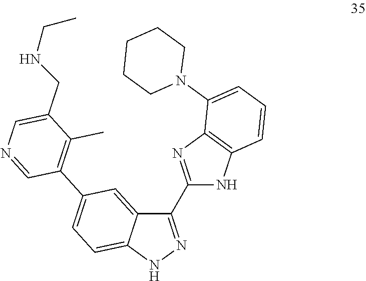

### N-((4-methyl-5-(3-(4-(piperidin-1-yl)-1H-benzo[d]imidazol-2-yl)-1H-indazol-5-yl)pyridin-3-yl)methyl)ethanamine 35

¹H NMR (DMSO-d₆) δ ppm 1.09 (t, 3H), 2.15-1.25 (m, 4H), 2.18 (s, 3H), 2.80 (m, 3H), 3.60 (m, 4H), 4.00 (s, 2H), 6.45 (s, 1H), 7.00-7.10 (m, 2H), 7.51 (m, 1H), 7.80 (m, 1H), 8.40-8.60 (m, 3H), ESIMS found for C₂₈H₃₁N₇ m/z 466 (M+H).

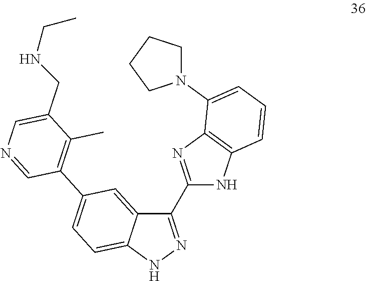

### N-((4-methyl-5-(3-(4-(pyrrolidin-1-yl)-1H-benzo[d]imidazol-2-yl)-1H-indazol-5-yl)pyridin-3-yl)methyl)ethanamine 36

¹H NMR (DMSO-d₆) δ ppm 1.15 (t, 3H), 2.10 (m, 4H), 2.38 (s, 3H), 2.80 (m, 2H), 3.78 (m, 4H), 3.99 (s, 2H), 6.20 (m, 1H), 6.78 (m, 1H), 7.09 (m, 1H), 7.30 (m, 1H), 7.78 (m, 1H), 8.40-8.60 (m, 3H), ESIMS found for C₂₇H₂₉N₇ m/z 452 (M+H).

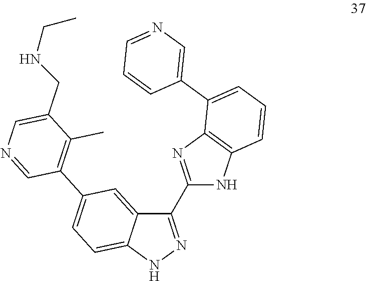

### N-((4-methyl-5-(3-(4-(pyridin-3-yl)-1H-benzo[d]imidazol-2-yl)-1H-indazol-5-yl)pyridin-3-yl)methyl)ethanamine 37

¹H NMR (MeOD) δ ppm 1.13 (t, 3H), 2.20 (s, 3H), 3.10 (m, 2H), 4.22 (m, 2H), 7.30-7.80 (m, 6H), 8.40-8.60 (m, 5H), 9.42 (s, 1H), ESIMS found for C₂₈H₂₅N₇ m/z 460 (M+H).

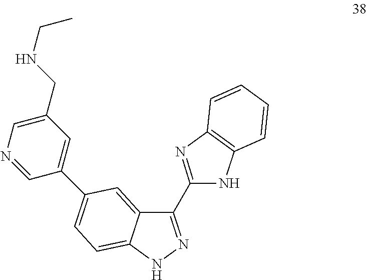

### N-((5-(3-(1H-benzo[d]imidazol-2-yl)-1H-indazol-5-yl)pyridin-3-yl)methyl)ethanamine 38

¹H NMR (DMSO-d₆) δ ppm 1.10 (t, 3H), 3.00 (m, 2H), 4.21 (s, 2H), 7.20 (m, 2H), 7.60 (m, 1H), 7.75-7.85 (m, 3H), 8.31 (m, 1H), 8.80-9.01 (m, 3H), ESIMS found for C₂₂H₂₀N₆ m/z 369 (M+H).

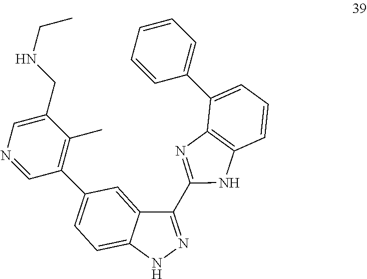

### N-((4-methyl-5-(3-(4-phenyl-1H-benzo[d]imidazol-2-yl)-1H-indazol-5-yl)pyridin-3-yl)methyl)ethanamine 39

¹H NMR (DMSO-d₆) δ ppm 1.15 (t, 3H), 2.36 (s, 3H), 3.00 (m, 2H), 4.20 (s, 2H), 7.20-7.60 (m, 6H), 7.82 (m, 1H), 8.31 (m, 2H), 8.50-8.70 (m, 3H), ESIMS found for C₂₉H₂₆N₆ m/z 459 (M+H).

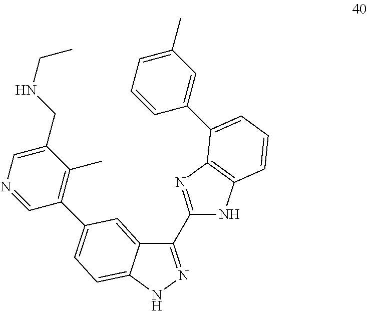

### N-((4-methyl-5-(3-(4-m-tolyl-1H-benzo[d]imidazol-2-yl)-1H-indazol-5-yl)pyridin-3-yl)methyl)ethanamine 40

¹H NMR (DMSO-d₆) δ ppm 1.09 (t, 3H), 2.18 (s, 3H), 2.36 (s, 3H), 2.70 (m, 2H), 3.84 (s, 2H), 7.00-7.60 (m, 6H), 7.75-7.95 (m, 2H), 8.23 (s, 1H), 8.60-8.85 (m, 3H), ESIMS found for C₃₀H₂₈N₆ m/z 473 (M+H).

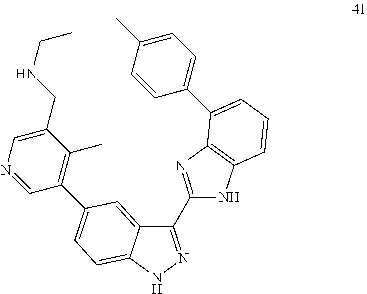

### N-((4-methyl-5-(3-(4-p-tolyl-1H-benzo[d]imidazol-2-yl)-1H-indazol-5-yl)pyridin-3-yl)methyl)ethanamine 41

¹H NMR (CD₃OD) δ ppm 1.40 (t, 3H), 2.40 (s, 3H), 2.44 (s, 3H), 2.70 (m, 2H), 4.44 (s, 2H), 7.30-7.42 (m, 4H), 7.48 (m, 1H), 7.66 (m, 1H), 7.76-7.90 (m, 23H), 8.27 (s, 1H), 8.61-8.72 (m, 2H), ESIMS found for C₃₀H₂₈N₆ m/z 473 (M+H).

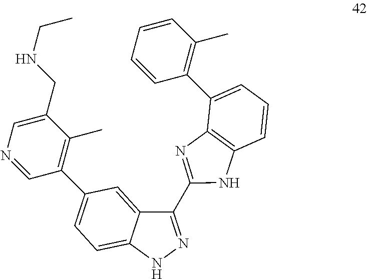

### N-((4-methyl-5-(3-(4-o-tolyl-1H-benzo[d]imidazol-2-yl)-1H-indazol-5-yl)pyridin-3-yl)methyl)ethanamine 42

¹H NMR (CD₃OD) δ ppm 1.31 (t, 3H), 2.26 (s, 3H), 2.40 (s, 3H), 2.81 (m, 2H), 3.98 (s, 2H), 7.17 (m, 1H), 7.30-7.45 (m, 5H), 7.60-7.80 (m, 2H), 8.10 (m, 1H), 8.38-8.60 (m, 3H), ESIMS found for C₃₀H₂₈N₆ m/z 473 (M+H).

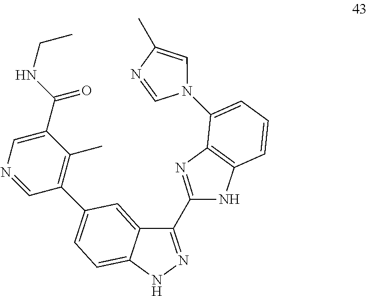

### N-ethyl-4-methyl-5-(3-(4-(4-methyl-1H-imidazol-1-yl)-1H-benzo[d]imidazol-2-yl)-1H-indazol-5-yl)nicotinamide 43

¹H NMR (DMSO-d₆) δ ppm 1.09 (t, 3H), 2.20 (s, 3H), 2.29 (s, 3H), 6.90-7.20 (m, 2H), 7.35 (m, 1H), 7.42-7.60 (m, 2H), 7.80-8.00 (m, 2H), 8.40-8.66 (m, 3H), 8.83 (m, 1H), ESIMS found for C₂₇H₂₄N₈O m/z 477 (M+H).

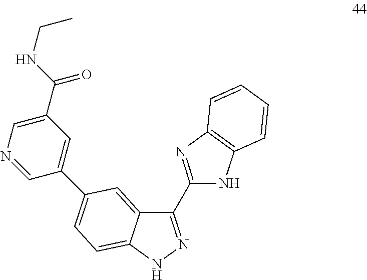

### 5-(3-(1H-benzo[d]imidazol-2-yl)-1H-indazol-5-yl)-N-ethyl nicotinamide 44

¹H NMR (DMSO-d₆) δ ppm 1.13 (t, 3H), 3.00 (m, 2H), 7.15 (s, 2H), 7.65 (m, 2H), 8.01 (m, 2H), 8.55 (m, 1H), 8.80 (m, 2H), 9.00-9.20 (m, 2H), ESIMS found for C₂₂H₁₈N₆O m/z 383 (M+H).

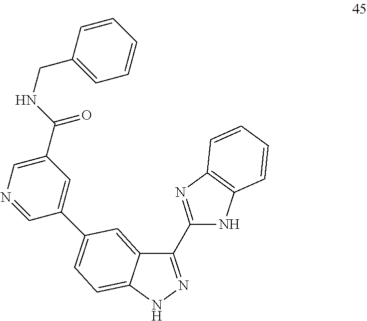

### 5-(3-(1H-benzo[d]imidazol-2-yl)-1H-indazol-5-yl)-N-benzylnicotinamide 45

¹H NMR (DMSO-d₆) δ ppm 4.60 (s, 2H), 6.95-7.15 (m, 1H), 7.20-7.40 (m, 6H) 7.60-8.00 (m, 3H), 8.60 (s, 1H), 8.80 (s, 1H), 9.10 (m, 2H), 9.43 (m, 1H), ESIMS found for C₂₇H₂₀N₆O m/z 445 (M+H).

### Administration and Pharmaceutical Compositions

Some embodiments include pharmaceutical compositions comprising: (a) a safe and therapeutically effective amount of a compound described herein, or its corresponding enantiomer, diastereoisomer or tautomer, or pharmaceutically acceptable salt; and (b) a pharmaceutically acceptable carrier.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarily, vaginally, rectally, or intraocularly. Oral and parenteral administrations are customary in treating the indications.

Compounds of the invention intended for pharmaceutical use may be administered as crystalline or amorphous products. Pharmaceutically acceptable compositions include solid, semi-solid, liquid and aerosol dosage forms, such as, e.g., tablets, capsules, powders, liquids, suspensions, suppositories, aerosols or the like. They may be obtained, for example, as films by methods such as precipitation, crystallization, freeze drying, spray drying, or evaporative drying. Microwave or radio frequency drying may be used for this purpose. The compounds can also be administered in sustained or controlled release dosage forms, including depot injections, osmotic pumps, pills, transdermal (including electrotransport) patches, and the like, for prolonged and/or timed, pulsed administration at a predetermined rate. Preferably, the compositions are provided in unit dosage forms suitable for single administration of a precise dose.

The compounds can be administered either alone or more typically in combination with a conventional pharmaceutical carrier, excipient or the like. The term “excipient” is used herein to describe any ingredient other than the compound(s) of the invention. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-b-cyclodextrins, or other solubilized derivatives can also be advantageously used to enhance delivery of compounds of the formulae described herein. Dosage forms or compositions containing a compound as described herein in the range of 0.005% to 100% with the balance made up from non-toxic carrier may be prepared. The contemplated compositions may contain 0.001%-100% active ingredient, in one embodiment 0.1-95%, in another embodiment 75-85%. Actual methods of preparing such dosage forms are known, or will be apparent, to those skilled in this art; for example, see Remington: The Science and Practice of Pharmacy, 21st Edition (Lippincott Williams & Wilkins. 2005).

In one preferred embodiment, the compositions will take the form of a unit dosage form such as a pill or tablet and thus the composition may contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule. Unit dosage forms in which the two active ingredients are physically separated are also contemplated; e.g., capsules with granules of each drug; two-layer tablets; two-compartment gel caps, etc.

Liquid pharmaceutically administrable compositions can, for example, be prepared by dissolving, dispersing, etc. an active compound as defined above and optional pharmaceutical adjuvants in a carrier (e.g., water, saline, aqueous dextrose, glycerol, glycols, ethanol or the like) to form a solution or suspension. If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Injectables can be prepared in conventional forms, either as liquid solutions or suspensions, as emulsions, or in solid forms suitable for dissolution or suspension in liquid prior to injection. The percentage of active compound contained in such parenteral compositions is highly dependent on the specific nature thereof, as well as the activity of the compound and the needs of the subject. However, percentages of active ingredient of 0.01% to 10% in solution are employable, and will be higher if the composition is a solid, which will be subsequently diluted to the above percentages. In some embodiments, the composition will comprise 0.2-2% of the active agent in solution.

It is to be noted that concentrations and dosage values may also vary with the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Solid compositions can be provided in various different types of dosage forms, depending on the physicochemical properties of the drug, the desired dissolution rate, cost considerations, and other criteria. In one of the embodiments, the solid composition is a single unit. This implies that one unit dose of the drug is comprised in a single, physically shaped solid form or article. In other words, the solid composition is coherent, which is in contrast to a multiple unit dosage form, in which the units are incoherent.

Examples of single units which may be used as dosage forms for the solid composition include tablets, such as compressed tablets, film-like units, foil-like units, wafers, lyophilized matrix units, and the like. In a preferred embodiment, the solid composition is a highly porous lyophilized form. Such lyophilizates, sometimes also called wafers or lyophilized tablets, are particularly useful for their rapid disintegration, which also enables the rapid dissolution of the active compound.

On the other hand, for some applications the solid composition may also be formed as a multiple unit dosage form as defined above. Examples of multiple units are powders, granules, microparticles, pellets, beads, lyophilized powders, and the like. In one embodiment, the solid composition is a lyophilized powder. Such a dispersed lyophilized system comprises a multitude of powder particles, and due to the lyophilization process used in the formation of the powder, each particle has an irregular, porous microstructure through which the powder is capable of absorbing water very rapidly, resulting in quick dissolution.

Another type of multiparticulate system which is also capable of achieving rapid drug dissolution is that of powders, granules, or pellets from water-soluble excipients which are coated with the drug, so that the drug is located at the outer surface of the individual particles. In this type of system, the water-soluble low molecular weight excipient is useful for preparing the cores of such coated particles, which can be subsequently coated with a coating composition comprising the drug and, preferably, one or more additional excipients, such as a binder, a pore former, a saccharide, a sugar alcohol, a film-forming polymer, a plasticizer, or other excipients used in pharmaceutical coating compositions.

Also provided herein are kits. Typically, a kit includes one or more compounds or compositions as described herein. In certain embodiments, a kit can include one or more delivery systems, e.g., for delivering or administering a compound as provided above, and directions for use of the kit (e.g., instructions for treating a patient). In another embodiment, the kit can include a compound or composition as described herein and a label that indicates that the contents are to be administered to a patient with cancer. In another embodiment, the kit can include a compound or composition as described herein and a label that indicates that the contents are to be administered to a patient with one or more of hepatocellular carcinoma, colon cancer, leukemia, lymphoma, sarcoma, ovarian cancer, diabetic retinopathy, neovascular glaucoma, rheumatoid arthritis, psoriasis, mycotic and viral infections, osteochondrodysplasia, Alzheimer's disease, osteoarthritis, polyposis coli, osteoporosis-pseudoglioma syndrome, familial exudative vitreoretinopathy, retinal angiogenesis, early coronary disease, tetra-amelia syndrome, Müllerian-duct regression and virilization, SERKAL syndrome, diabetes mellitus type 2, Fuhrmann syndrome, Al-Awadi/Raas-Rothschild/Schinzel phocomelia syndrome, odonto-onycho-dermal dysplasia, obesity, split-hand/foot malformation, caudal duplication syndrome, tooth agenesis, Wilms tumor, skeletal dysplasia, focal dermal hypoplasia, autosomal recessive anonychia, neural tube defects, alpha-thalassemia (ATRX) syndrome, fragile X syndrome, ICF syndrome, Angelman syndrome, Prader-Willi syndrome, Beckwith-Wiedemann Syndrome and Rett syndrome.

The actual dose of the active compounds of the present invention depends on the specific compound, and on the condition to be treated; the selection of the appropriate dose is well within the knowledge of the skilled artisan.

### Methods of Treatment

The compounds and compositions provided herein can be used as inhibitors of one or more members of the Wnt pathway, including one or more Wnt proteins, and thus can be used to treat a variety of disorders and diseases in which aberrant Wnt signaling is implicated, such as cancer and other diseases associated with abnormal angiogenesis, cellular proliferation, and cell cycling. Accordingly, the compounds and compositions provided herein can be used to treat cancer, to reduce or inhibit angiogenesis, to reduce or inhibit cellular proliferation and correct an genetic disorder due to mutations in Wnt signaling components. Non-limiting examples of diseases which can be treated with the compounds and compositions provided herein include a variety of cancers, diabetic retinopathy, neovascular glaucoma, rheumatoid arthritis, psoriasis, mycotic and viral infections, osteochondrodysplasia, Alzheimer's disease, osteoarthritis, polyposis coli, osteoporosis-pseudoglioma syndrome, familial exudative vitreoretinopathy, retinal angiogenesis, early coronary disease, tetra-amelia syndrome, Müllerian-duct regression and virilization, SERKAL syndrome, diabetes mellitus type 2, Fuhrmann syndrome, Al-Awadi/Raas-Rothschild/Schinzel phocomelia syndrome, odonto-onycho-dermal dysplasia, obesity, split-hand/foot malformation, caudal duplication syndrome, tooth agenesis, Wilms tumor, skeletal dysplasia, focal dermal hypoplasia, autosomal recessive anonychia, neural tube defects, alpha-thalassemia (ATRX) syndrome, fragile X syndrome, ICF syndrome, Angelman syndrome, Prader-Willi syndrome, Beckwith-Wiedemann Syndrome and Rett syndrome.

With respect to cancer, the Wnt pathway is known to be constitutively activated in a variety of cancers including, for example, colon cancer, hepatocellular carcinoma, lung cancer, ovarian cancer, prostate cancer, pancreatic cancer and leukemias such as CML, CLL and T-ALL. The constitutive activation is due to constitutively active β-catenin, perhaps due to its stabilization by interacting factors or inhibition of the degradation pathway. Accordingly, the compounds and compositions described herein may be used to treat these cancers in which the Wnt pathway is constitutively activated. In certain embodiments, the cancer is chosen from hepatocellular carcinoma, colon cancer, leukemia, lymphoma, sarcoma and ovarian cancer.

Other cancers can also be treated with the compounds and compositions described herein.

More particularly, cancers that may be treated by the compound, compositions and methods described herein include, but are not limited to, the following:

1) Breast cancers, including, for example ER⁺ breast cancer, ER⁻ breast cancer, her2⁻ breast cancer, her2⁺ breast cancer, stromal tumors such as fibroadenomas, phyllodes tumors, and sarcomas, and epithelial tumors such as large duct papillomas; carcinomas of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma; and miscellaneous malignant neoplasms. Further examples of breast cancers can include luminal A, luminal B, basal A, basal B, and triple negative breast cancer, which is estrogen receptor negative (ER⁻), progesterone receptor negative, and her2 negative (her2⁻). In some embodiments, the breast cancer may have a high risk Oncotype score.

2) Cardiac cancers, including, for example sarcoma, e.g., angiosarcoma, fibrosarcoma, rhabdomyosarcoma, and liposarcoma; myxoma; rhabdomyoma; fibroma; lipoma and teratoma.

3) Lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; and mesothelioma.

4) Gastrointestinal cancer, including, for example, cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma.

Genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma.

6) Liver cancers, including, for example, hepatoma, e.g., hepatocellular carcinoma; cholangiocarcinoma; hepatoblastoma; angiosarcoma; hepatocellular adenoma; and hemangioma.

7) Bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors.

8) Nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, schwannoma, retinoblastoma, and congenital tumors; and cancers of the spinal cord, e.g., neurofibroma, meningioma, glioma, and sarcoma.

9) Gynecological cancers, including, for example, cancers of the uterus, e.g., endometrial carcinoma; cancers of the cervix, e.g., cervical carcinoma, and pre tumor cervical dysplasia; cancers of the ovaries, e.g., ovarian carcinoma, including serous cystadenocarcinoma, mucinous cystadenocarcinoma, unclassified carcinoma, granulosa theca cell tumors, Sertoli Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma.

10) Hematologic cancers, including, for example, cancers of the blood, e.g., acute myeloid leukemia, chronic myeloid leukemia, acute lymphoblastic leukemia, chronic lymphocytic leukemia, myeloproliferative diseases, multiple myeloma, and myelodysplastic syndrome, Hodgkin's lymphoma, non Hodgkin's lymphoma (malignant lymphoma) and Waldenstrom's macroglobulinemia.

11) Skin cancers and skin disorders, including, for example, malignant melanoma, basal cell carcinoma, squamous cell carcinoma, Kaposi's sarcoma, moles dysplastic nevi, lipoma, angioma, dermatofibroma, keloids, and psoriasis.

12) Adrenal gland cancers, including, for example, neuroblastoma.

Cancers may be solid tumors that may or may not be metastatic. Cancers may also occur, as in leukemia, as a diffuse tissue. Thus, the term “tumor cell,” as provided herein, includes a cell afflicted by any one of the above identified disorders.

A method of treating cancer using a compound or composition as described herein may be combined with existing methods of treating cancers, for example by chemotherapy, irradiation, or surgery (e.g., oophorectomy). In some embodiments, a compound or composition can be administered before, during, or after another anticancer agent or treatment.

The compounds and compositions described herein can be used as anti-angiogenesis agents and as agents for modulating and/or inhibiting the activity of protein kinases, thus providing treatments for cancer and other diseases associated with cellular proliferation mediated by protein kinases. For example, the compounds described herein can inhibit the activity of one or more kinases, such as CDKs, VEGF, CLK, JAK, HIPK, Abl and CHK-1, or cyclin complexes thereof. Accordingly, provided herein is a method of treating cancer or preventing or reducing angiogenesis through kinase inhibition, such as through inhibition of VEGF, CLK, HIPK, Abl CHK-1, CDK4 or CDK4/D-type cyclin complexes and/or CDK2 or CDK2/E-type cyclin complexes.

In addition, and including treatment of cancer, the compounds and compositions described herein can function as cell-cycle control agents for treating proliferative disorders in a patient. Disorders associated with excessive proliferation include, for example, cancers, psoriasis, immunological disorders involving undesired proliferation of leukocytes, and restenosis and other smooth muscle disorders. Furthermore, such compounds may be used to prevent de-differentiation of post-mitotic tissue and/or cells.

Diseases or disorders associated with uncontrolled or abnormal cellular proliferation include, but are not limited to, the following:
a variety of cancers, including, but not limited to, carcinoma, hematopoietic tumors of lymphoid lineage, hematopoietic tumors of myeloid lineage, tumors of mesenchymal origin, tumors of the central and peripheral nervous system and other tumors including melanoma, seminoma and Kaposi's sarcoma.
a disease process which features abnormal cellular proliferation, e.g., benign prostatic hyperplasia, familial adenomatosis polyposis, neuro-fibromatosis, atherosclerosis, pulmonary fibrosis, arthritis, psoriasis, glomerulonephritis, restenosis following angioplasty or vascular surgery, hypertrophic scar formation, inflammatory bowel disease, transplantation rejection, endotoxic shock, and fungal infections.
defective apoptosis-associated conditions, such as cancers (including but not limited to those types mentioned hereinabove), viral infections (including but not limited to herpesvirus, poxvirus, Epstein-Barr virus, Sindbis virus and adenovirus), prevention of AIDS development in HIV-infected individuals, autoimmune diseases (including but not limited to systemic lupus erythematosus, rheumatoid arthritis, psoriasis, autoimmune mediated glomerulonephritis, inflammatory bowel disease and autoimmune diabetes mellitus), neurodegenerative disorders (including but not limited to Alzheimer's disease, amyotrophic lateral sclerosis, retinitis pigmentosa, Parkinson's disease, AIDS-related dementia, spinal muscular atrophy and cerebellar degeneration), myelodysplastic syndromes, aplastic anemia, ischemic injury associated with myocardial infarctions, stroke and reperfusion injury, arrhythmia, atherosclerosis, toxin-induced or alcohol related liver diseases, hematological diseases (including but not limited to chronic anemia and aplastic anemia), degenerative diseases of the musculoskeletal system (including but not limited to osteoporosis and arthritis), aspirin-sensitive rhinosinusitis, cystic fibrosis, multiple sclerosis, kidney diseases and cancer pain.
genetic diseases due to mutations in Wnt signaling components, such as polyposis coli, osteoporosis-pseudoglioma syndrome, familial exudative vitreoretinopathy, retinal angiogenesis, early coronary disease, tetra-amelia syndrome, Müllerian-duct regression and virilization, SERKAL syndrome, diabetes mellitus type 2, Fuhrmann syndrome, Al-Awadi/Raas-Rothschild/Schinzel phocomelia syndrome, odonto-onycho-dermal dysplasia, obesity, split-hand/foot malformation, caudal duplication syndrome, tooth agenesis, Wilms tumor, skeletal dysplasia, focal dermal hypoplasia, autosomal recessive anonychia, neural tube defects, alpha-thalassemia (ATRX) syndrome, fragile X syndrome, ICF syndrome, Angelman syndrome, Prader-Willi syndrome, Beckwith-Wiedemann Syndrome and Rett syndrome.

The compounds and compositions may also be useful in the inhibition of the development of invasive cancer, tumor angiogenesis and metastasis.

Moreover, the compounds and compositions, for example, as inhibitors of the CDKs, can modulate the level of cellular RNA and DNA synthesis and therefore are expected to be useful in the treatment of viral infections such as HIV, human papilloma virus, herpes virus, Epstein-Barr virus, adenovirus, Sindbis virus, pox virus and the like.

Compounds and compositions described herein can inhibit the kinase activity of, for example, CDK/cyclin complexes, such as those active in the G₀. or G.₁ stage of the cell cycle, e.g., CDK2, CDK4, and/or CDK6 complexes.

### Evaluation of Biological Activity

The biological activity of the compounds described herein can be tested using any suitable assay known to those of skill in the art, e.g., WO 2001/053268 or WO 2005/009997. For example, the activity of a compound may be tested using one or more of the test methods outlined below.

In one example, tumor cells may be screened for Wnt independent growth. In such a method, tumor cells of interest are contacted with a compound (i.e. inhibitor) of interest, and the proliferation of the cells, e.g. by uptake of tritiated thymidine, is monitored. In some embodiments, tumor cells may be isolated from a candidate patient who has been screened for the presence of a cancer that is associated with a mutation in the Wnt signaling pathway. Candidate cancers include, without limitation, those listed above.

In another example, one may utilize in vitro assays for Wnt biological activity, e.g. stabilization of β-catenin and promoting growth of stem cells. Assays for biological activity of Wnt include stabilization of β-catenin, which can be measured, for example, by serial dilutions of a candidate inhibitor composition. An exemplary assay for Wnt biological activity contacts a Wnt composition in the presence of a candidate inhibitor with cells, e.g. mouse L cells. The cells are cultured for a period of time sufficient to stabilize β-catenin, usually at least about 1 hour, and lysed. The cell lysate is resolved by SDS PAGE, then transferred to nitrocellulose and probed with antibodies specific for β-catenin.

In a further example, the activity of a candidate compound can be measured in a Xenopus secondary axis bioassay (Leyns, L. et al. Cell (1997), 88(6), 747-756).

### Example 5

Another screening assay for Wnt activity is described as follows. Reporter cell lines can be generated by stably transducing cells of cancer cell lines (e.g., colon cancer) with a lentiviral construct that include a wnt-responsive promoter driving expression of the firefly luciferase gene.

Lentiviral constructs can be made in which the SP5 promoter, a promoter having eight TCF/LEF binding sites derived from the SP5 promoter, is linked upstream of the firefly luciferase gene. The lentiviral constructs can also include a hygromycin resistance gene as a selectable marker. The SP5 promoter construct can be used to transduce SW480 cells, a colon cancer cell line having a mutated APC gene that generates a truncated APC protein, leading to de-regulated accumulation of β-catenin. A control cell line can be generated using another lentiviral construct containing the luciferase gene under the control of the SV40 promoter which does not require β-catenin for activation.

Cultured SW480 cells bearing a reporter construct can be distributed at approximately 10,000 cells per well into 384 well multiwell plates. Compounds from a small molecule compound library can then be added to the wells in half-log dilutions using a three micromolar top concentration. A series of control wells for each cell type receive only buffer and compound solvent. Twenty-four hours after the addition of compound, reporter activity for luciferases can be assayed, for example, by addition of the BrightGlo luminescence reagent (Promega) and the Victor3 plate reader (Perkin Elmer). Readings can be normalized to DMSO only treated cells, and normalized activities can then be used in the IC₅₀ calculations. Table 5 shows the activity of selected compounds described herein.

TABLE 5

| Compound | Wnt inhibition, IC₅₀ | |
|---|---|---|
| 1 | 200-310 | nM |
| 2 | 500 | nM |
| 3 | 580 | nM |
| 4 | 600 | nM |
| 5 | 630 | nM |
| 7 | 800 | nM |
| 8 | 930 | nM |
| 9 | 1 | μM |
| 10 | 1 | μM |
| 11 | 1.3 | μM |
| 12 | 1.6 | μM |
| 13 | 1.8 | μM |
| 14 | 1.9 | μM |
| 15 | 3 | μM |
| 16 | 3.1 | μM |
| 17 | 750 | nM |
| 18 | 1.9 | μM |
| 19 | 330-400 | nM |
| 20 | 606 | nM |
| 21 | 5.3 | μM |
| 22 | 1.85 | μM |
| 23 | >10 | μM |
| 24 | 8 | μM |
| 25 | >10 | μM |
| 26 | >10 | μM |
| 27 | >10 | μM |
| 28 | 1 | μM |
| 29 | >10 | μM |
| 30 | 2 | μM |
| 31 | 243 | nM |
| 32 | 40-63 | nM |
| 33 | 680 | nM |
| 34 | 10-46 | nM |
| 35 | 147-243 | nM |
| 36 | 750 | nM |
| 37 | 200-240 | nM |
| 38 | 124-235 | nM |
| 39 | 690-730 | nM |
| 40 | 670-860 | nM |
| 41 | 330-470 | nM |
| 42 | 2.5-3.6 | μM |
| 43 | 77-96 | nM |
| 44 | 0.96-1.56 | μM |
| 45 | 1.23-2.28 | μM |
| 48 | 390 | nM |
| 123 | 1.22 | μM |

The term “comprising” as used herein is synonymous with “including,” “containing,” or “characterized by,” and is inclusive or open-ended and does not exclude additional, unrecited elements or method steps.

What is claimed is:

1. A method of treating a disorder or disease in which aberrant Wnt signaling is implicated in a patient, the method comprising administering to the patient a therapeutically effective amount of a compound having a structure of Formula I, or a pharmaceutically acceptable salt or pro-drug thereof: 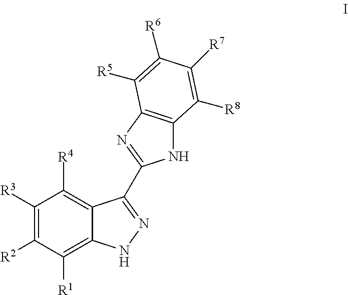
wherein R¹, R², R⁴, R⁵, R⁶, R⁷ and R⁸ are independently selected from the group consisting of H, C₁₋₉ alkyl, halide, —CF₃, —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)S(═O)R¹⁰, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹;
R³ is selected from the group consisting of —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)S(═O)R¹⁰, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹;
alternatively, one of each R¹ and R², R² and R³, R³ and R⁴, R⁵ and R⁶, R⁶ and R⁷ or R⁷ and R⁸ are taken together to form a ring which is selected from the group consisting of aryl, heteroaryl, 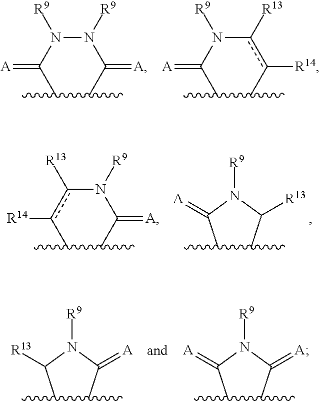
wherein each bond represented by a dashed and solid line represents a bond selected from the group consisting of a single bond and a double bond;
each R⁹ is independently selected from the group consisting of H, —C₁₋₉ alkyl, —CF₃, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;
each R¹⁰ is independently selected from the group consisting of —C₁₋₉ alkyl, —CF₃, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;
each R¹¹ is independently selected from the group consisting of CN, —OR⁹ and R⁹;
each R¹² is 1-5 substituents each selected from the group consisting of H, —C₁₋₉ alkyl, -alkylaminoalkyl, halide, —CF₃, carbocyclylR¹², heterocyclylR¹², arylR¹², heteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙNR⁹C(═O)OR⁹, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹;
R¹³ and R¹⁴ are independently selected from the group consisting of H, C₁₋₉ alkyl, halide, —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙN(R⁹)C(=A)R⁹, —NO₂, —CN, —(C₁₋₉ alkyl)ₙCO₂R⁹ and —(C₁₋₉ alkyl)ₙC(=A)R⁹;
alternatively, R¹³ and R¹⁴ are taken together to form a ring which is selected from the group consisting of benzene and pyridine;
each A is independently selected from O, S and NR¹¹; and
each n is 0 or 1.

2. The method of claim 1 wherein n is 0.

3. The method of claim 1 wherein n is 1.

4. The method of claim 1 wherein A is O.

5. The method of claim 1 wherein R¹, R² and R⁴ are H and R³ is independently selected from the group consisting of —(C₁₋₉ alkyl)ₙarylR¹², —(C₁₋₉ alkyl)ₙheteroarylR¹², —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, —(C₁₋₉ alkyl)ₙOC(═O)N(R⁹)₂, —CN, n is 0 and A is O.

6. The method of claim 5 wherein R³ is arylR¹².

7. The method of claim 5 wherein R³ is heteroarylR¹².

8. The method of claim 5 wherein R³ is —N(R⁹)C(═O)N(R⁹)₂.

9. The method of claim 5 wherein R³ is —NR⁹C(═O)OR⁹.

10. The method of claim 5 wherein R³ is —C(═O)N(R⁹)₂.

11. The method of claim 5 wherein R³ is —N(R⁹)C(═O)R⁹.

12. The method of claim 5 wherein R³ is —OC(═O)N(R⁹)₂.

13. The method of claim 5 wherein R³ is —CN.

14. The method of claim 7 wherein the heteroarylR¹² is a pyridine and R¹² is selected from the group consisting of -alkylaminoalkyl, —C(═O)NHR⁹ and —NHC(═O)R⁹.

15. The method of claim 10 wherein R³ is —C(═O)NHR⁹ and R⁹ is selected from the group consisting of —C₁₋₉ alkyl and —(C₁₋₉ alkyl)ₙheterocyclylR¹².

16. The method of claim 1, wherein the compound has a structure selected from the group consisting of: 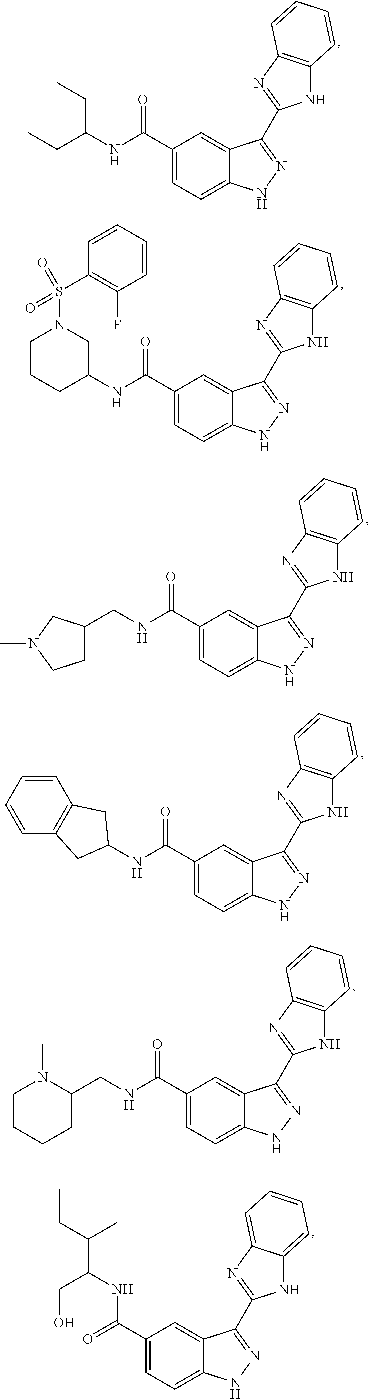 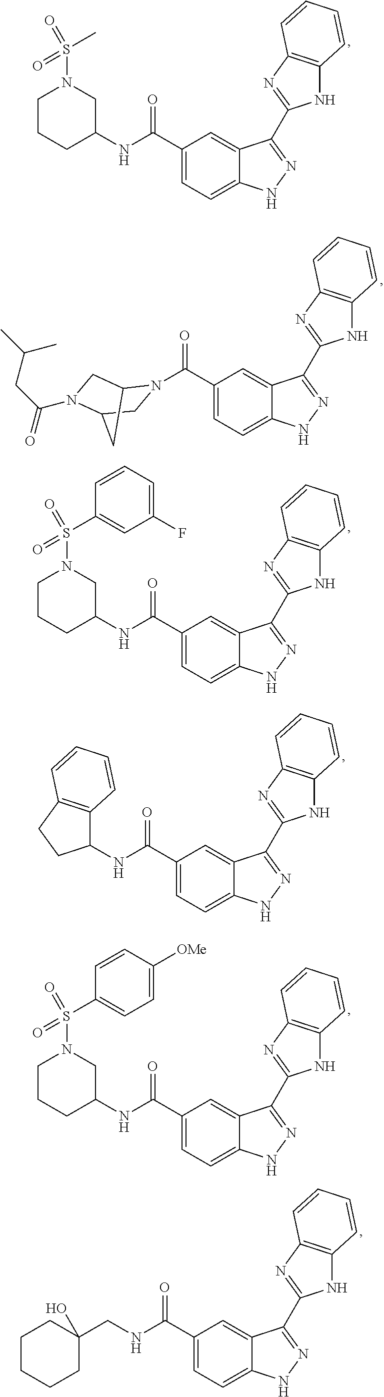 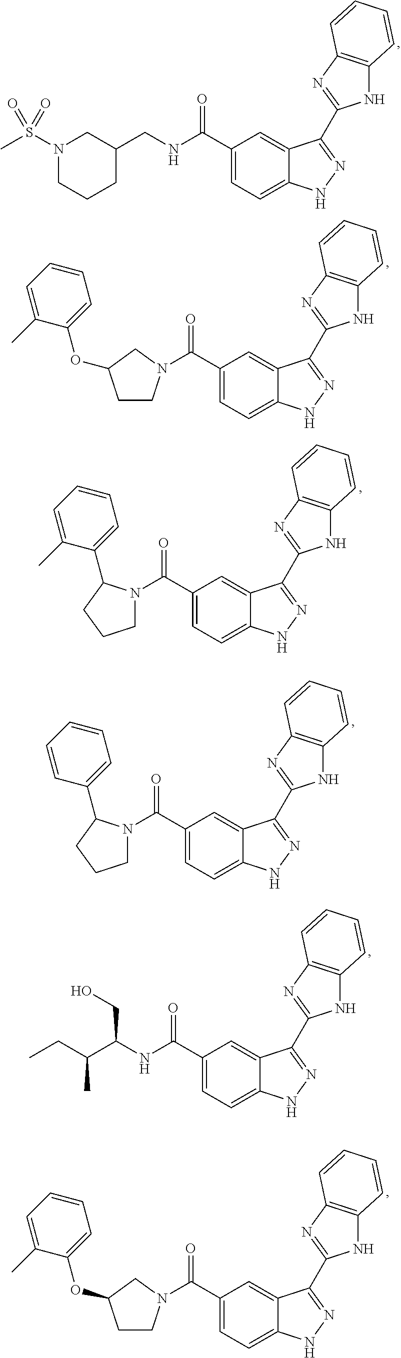 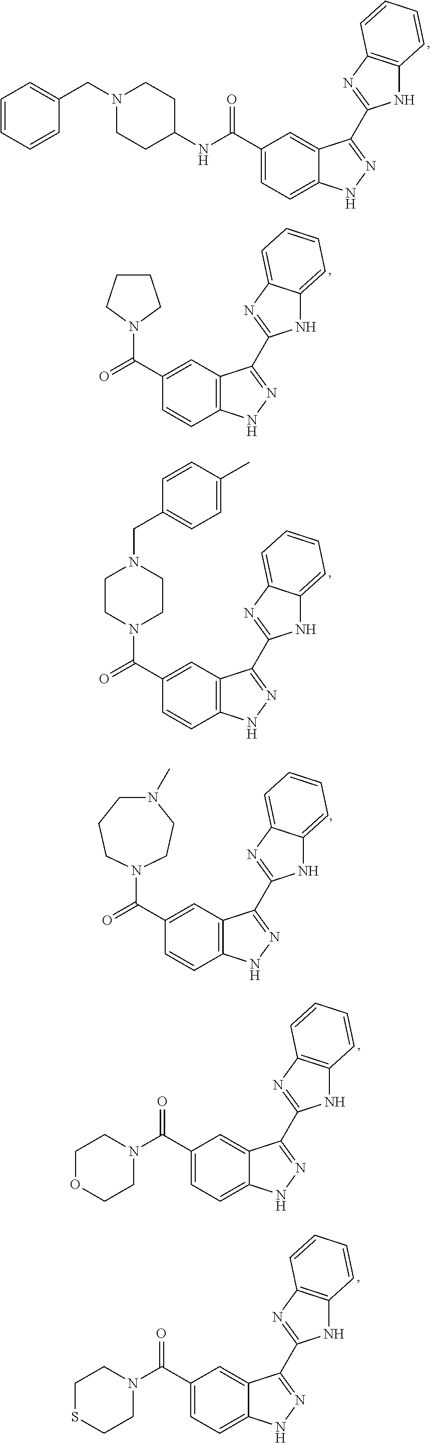 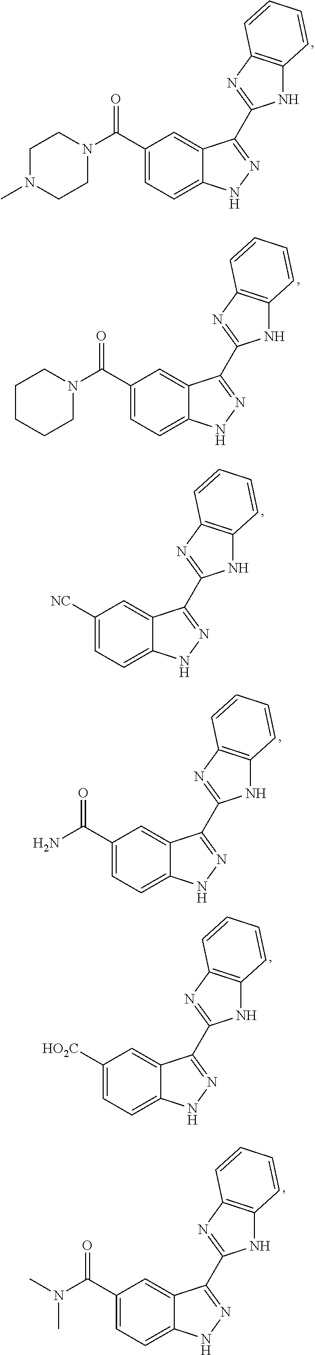 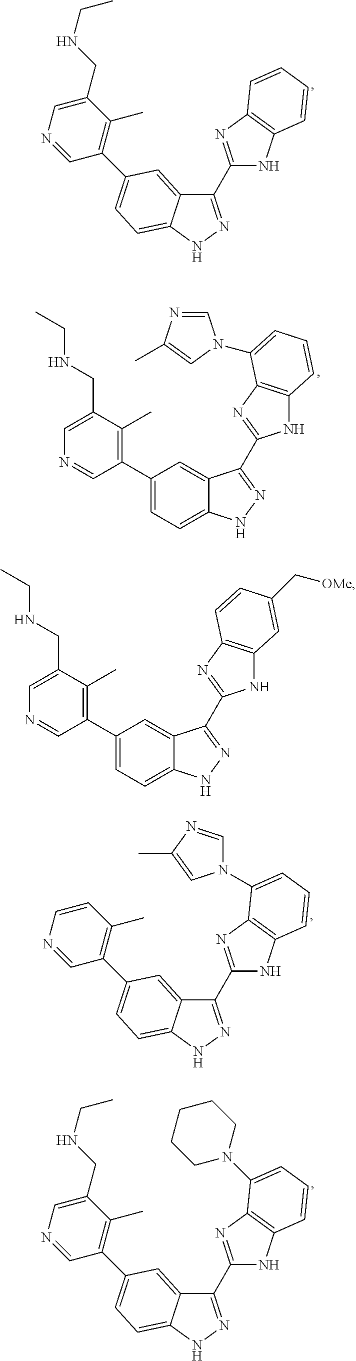 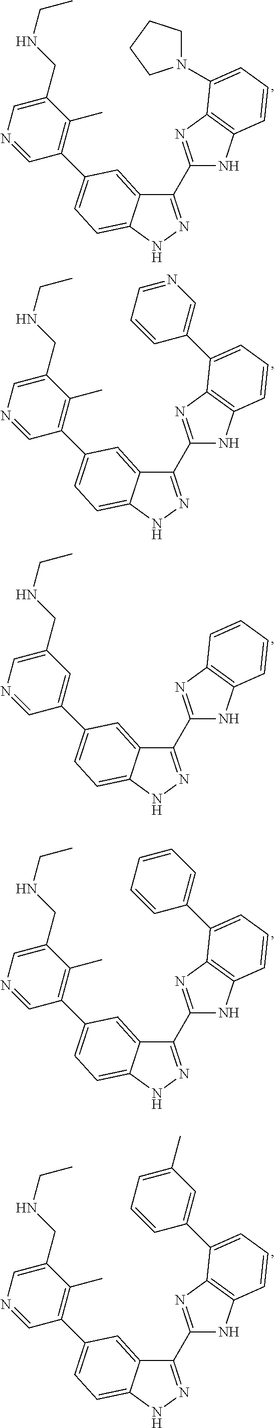 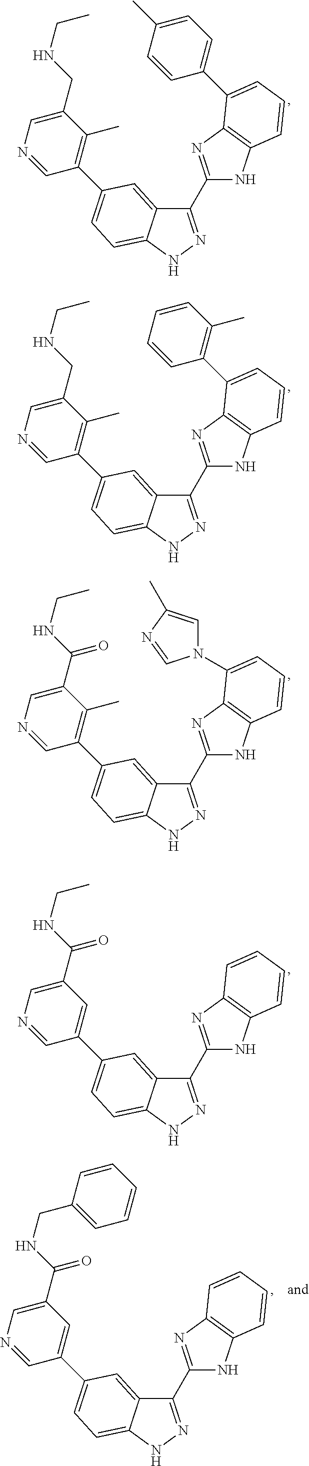 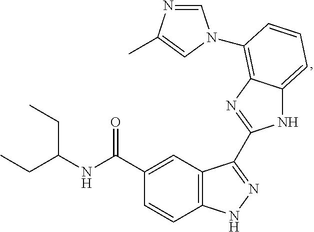
or a pharmaceutically acceptable salt or prodrug thereof.

17. A method of treating a disorder or disease in which aberrant Wnt signaling is implicated in a patient, the method comprising administering to the patient a therapeutically effective amount of a compound having a structure of Formula II, or a pharmaceutically acceptable salt or pro-drug thereof: 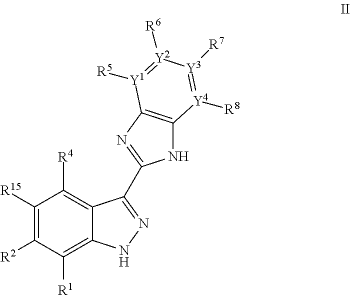
wherein R¹, R², R⁴, R⁵, R⁶, R⁷ and R⁸ are independently selected from the group consisting of H, C₁₋₉ alkyl, halide, —CF₃, —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)S(═O)R¹⁰, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹;
alternatively, one of each R¹ and R², R² and R¹⁵, R¹⁵ and R⁴, R⁵ and R⁶, R⁶ and R⁷ or R⁷ and R⁸ are taken together to form a ring which is selected from the group consisting of aryl, heteroaryl, 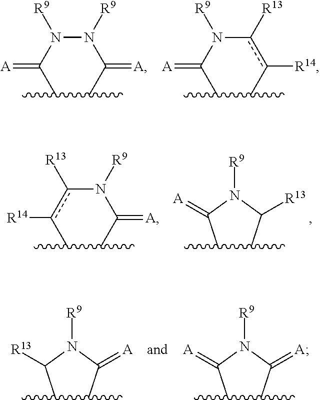
wherein each bond represented by a dashed and solid line represents a bond selected from the group consisting of a single bond and a double bond;
each R⁹ is independently selected from the group consisting of H, —C₁₋₉ alkyl, —CF₃, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;
each R¹⁰ is independently selected from the group consisting of —C₁₋₉ alkyl, —CF₃, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;
each R¹¹ is independently selected from the group consisting of CN, —OR⁹ and R⁹;
each R¹² is 1-5 substituents each selected from the group consisting of H, C₁₋₉ alkyl, -alkylaminoalkyl, halide, —CF₃, carbocyclylR¹², heterocyclylR¹², arylR¹², heteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙNR⁹C(═O)OR⁹, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹;
R¹³ and R¹⁴ are independently selected from the group consisting of H, C₁₋₉ alkyl, halide, —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙN(R⁹)C(=A)R⁹, —NO₂, —CN, —(C₁₋₉ alkyl)ₙCO₂R⁹ and —(C₁₋₉ alkyl)ₙC(=A)R⁹;
alternatively, R¹³ and R¹⁴ are taken together to form a ring which is selected from the group consisting of benzene and pyridine;
each R¹⁵ is selected from the group consisting of -carbocyclylR¹⁶, —(C₁₋₉ alkyl)carbocyclylR¹², -heterocyclylR¹⁶, —(C₁₋₉ alkyl)heterocyclylR¹², -arylR¹⁶, —(C₁₋₉ alkyl)arylR¹², -heteroarylR¹⁶, —(C₁₋₉ alkyl)heteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)N(R⁹)S(═O)R¹⁰, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, —NO₂, —CN, —(C₁₋₉ alkyl)ₙCO₂R⁹ and —(C₁₋₉ alkyl)ₙC(=A)R⁹;
each R¹⁶ is 1-5 substituents each selected from the group consisting of H, C₁₋₉ alkyl, halide, —CF₃, carbocyclylR¹², heterocyclylR¹², arylR¹², heteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)N(R⁹)SO₂R⁹, —(C₁₋₉ alkyl)ₙSO₂N(R⁹)₂, —N(R¹⁷)₂, —(C₁₋₉ alkyl)N(R⁹)₂, —NR⁹C(═O)N(R¹⁷)₂, —(C₁₋₉ alkyl)N(R⁹)C(=A)N(R⁹)₂, —C(═O)NR⁹R¹⁸, —(C₁₋₉ alkyl)C(=A)N(R⁹)₂, —(C₁₋₉ alkyl)NR⁹C(═O)OR⁹, —(C₁₋₉ alkyl)N(R⁹)C(=A)R⁹, —(C₁₋₉ alkyl)OC(═O)N(R⁹)₂, —NO₂, —CN, —(C₁₋₉ alkyl)ₙCO₂R⁹ and —(C₁₋₉ alkyl)C(=A)R⁹;
each R¹⁷ is H, —(C₁₋₉ alkyl)ₙcarbocyclyl, —(C₁₋₉ alkyl)ₙheterocyclyl, —(C₁₋₉ alkyl)ₙaryl and —(C₁₋₉ alkyl)ₙheteroaryl;
each R¹⁸ is independently selected from the group consisting of H, —C₁₋₉ alkyl, —CF₃, -carbocyclylR¹², —(C₁₋₉ alkyl)ₙarylR¹² and —(C₁₋₉ alkyl)ₙheteroarylR¹²;
each A is independently selected from O, S and NR¹¹;
Y¹, Y², Y³ and Y⁴ are independently selected from the group consisting of carbon and nitrogen with the proviso that at least one of Y¹, Y², Y³ and Y⁴ are nitrogen; wherein
if Y¹ is nitrogen then R⁵ is absent;
if Y² is nitrogen then R⁶ is absent;
if Y³ is nitrogen then R⁷ is absent;
if Y⁴ is nitrogen then R⁸ is absent; and
each n is 0 or 1.

18. The method of claim 17 wherein n is 0.

19. The method of claim 17 wherein n is 1.

20. The method of claim 17 wherein A is O.

21. The method of claim 17 wherein R¹, R² and R⁴ are H and R¹⁵ is independently selected from the group consisting of -heteroarylR¹⁶, -arylR¹⁶, —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, n is 0 and A is O.

22. The method of claim 21 wherein R¹⁵ is arylR¹⁶.

23. The method of claim 21 wherein R¹⁵ is heteroarylR¹⁶.

24. The method of claim 21 wherein R¹⁵ is —N(R⁹)C(═O)N(R⁹)₂.

25. The method of claim 21 wherein R¹⁵ is —NR⁹C(═O)OR⁹.

26. The method of claim 21 wherein R¹⁵ is —C(═O)N(R⁹)₂.

27. The method of claim 21 wherein R¹⁵ is —N(R⁹)C(═O)R⁹.

28. The method of claim 23 wherein the heteroarylR¹⁶ is a pyridine and R¹⁶ is selected from the group consisting of —(C₁₋₉ alkyl)NHR⁹ and —C(═O)NHR¹⁸.

29. The method of claim 26 wherein R¹⁵ is —C(═O)NHR⁹ and R⁹ is selected from the group consisting of —C₁₋₉ alkyl and —(C₁₋₉ alkyl)ₙheterocyclylR¹².

30. The method of claim 17, wherein the compound has the structure set forth below, or a pharmaceutically acceptable salt or prodrug thereof:

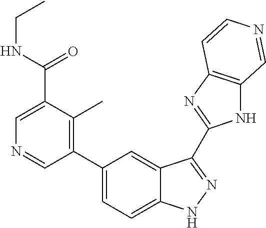

31. A method of treating a disorder or disease in which aberrant Wnt signaling is implicated in a patient, the method comprising administering to the patient a therapeutically effective amount of a compound having a structure of Formula III, or a pharmaceutically acceptable salt or pro-drug thereof: 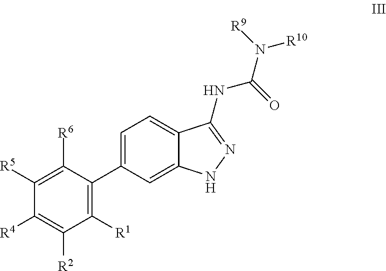
wherein R¹, R², R⁴, R⁵ and R⁶ are independently selected from the group consisting of H, C₁₋₉ alkyl, halide, —CF₃, —(C₁₋₉ alkyl)ₙcarbocyclylR¹², —(C₁₋₉ alkyl)ₙheterocyclylR¹², —(C₁₋₉ alkyl)ₙarylR¹², —(C₁₋₉ alkyl)ₙheteroarylR¹², —(C₁₋₉ alkyl)ₙOR⁹, —(C₁₋₉ alkyl)ₙSR⁹, —(C₁₋₉ alkyl)ₙS(═O)R¹⁰, —(C₁₋₉ alkyl)ₙSO₂R⁹, —(C₁₋₉ alkyl)ₙN(R⁹)S(═O)R¹⁰, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙNR⁹C(═O)OR⁹, —(C₁₋₉ alkyl)ₙC(=A)N(R⁹)₂, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹;
each R⁹ is independently selected from the group consisting of H, —C₁₋₉ alkyl, —(C₁₋₉ alkyl)OR⁹, —(C₁₋₉ alkyl)N(R⁹)₂, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;
each R¹⁰ is independently selected from the group consisting of —C₁₋₉ alkyl, —(C₁₋₉ alkyl)OR⁹, —(C₁₋₉ alkyl)N(R⁹)₂, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;
alternatively, R⁹ and R¹⁰ are taken together to form a 3-10 membered heterocyclyl ring;
each R¹¹ is independently selected from the group consisting of CN, —OR⁹ and R⁹;
each R¹² is 1-5 substituents each selected from the group consisting of H, C₁₋₉alkyl, -alkylaminoalkyl, halide, —CF₃, carbocyclylR¹², heterocyclylR¹², arylR¹², heteroarylR¹², —(C₁₋₉ alkyl)ₙOR⁹, —(C₁₋₉alkyl)ₙSR⁹, —(C₁₋₉alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙNR⁹C(═O)OR⁹, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹;
each A is independently selected from O, S and NR¹¹; and
each n is 0 or 1.

32. The method of claim 31 wherein n is 0.

33. The method of claim 31 wherein n is 1.

34. The method of claim 31 wherein A is O.

35. The method of claim 31 wherein R¹, R², R⁴, R⁵ and R⁶ are independently selected from the group consisting of independently selected from the group consisting of H, C₁₋₉ alkyl, halide, —CF₃, —(C₁₋₉ alkyl)ₙOR⁹, —(C₁₋₉ alkyl)ₙSR⁹, —(C₁₋₉ alkyl)ₙN(R⁹)₂, —CN and —(C₁₋₉ alkyl)ₙC(=A)R⁹.

36. The method of claim 31 wherein R⁹ is H.

37. The method of claim 31 wherein R¹⁰ is —C₁₋₉ alkyl.

38. The method of claim 31 wherein R¹⁰ is —(C₁₋₉ alkyl)OR⁹.

39. The method of claim 31 wherein R¹⁰ is —(C₁₋₉ alkyl)N(R⁹)₂.

40. The method of claim 31 wherein R¹⁰ is —(C₁₋₉ alkyl)carbocyclylR¹².

41. The method of claim 31 wherein R¹⁰ is —(C₁₋₉ alkyl)heterocyclylR¹².

42. The method of claim 31 wherein R¹⁰ is —(C₁₋₉ alkyl)arylR¹².

43. The method of claim 31 wherein R¹⁰ is —(C₁₋₉ alkyl)heteroarylR¹².

44. The method of claim 31 wherein R⁹ and R¹⁰ are taken together to form a 6-membered heterocyclyl ring.

45. The method of claim 31 wherein R⁹ and R¹⁰ are taken together to form a 5-membered heterocyclyl ring.

46. The method of claim 31, wherein the compound has a structure selected from the group consisting of: 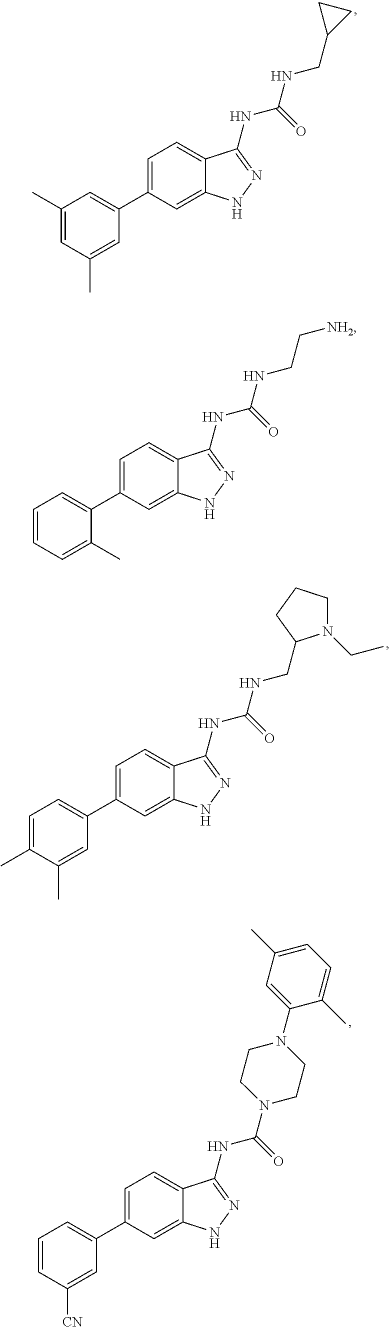 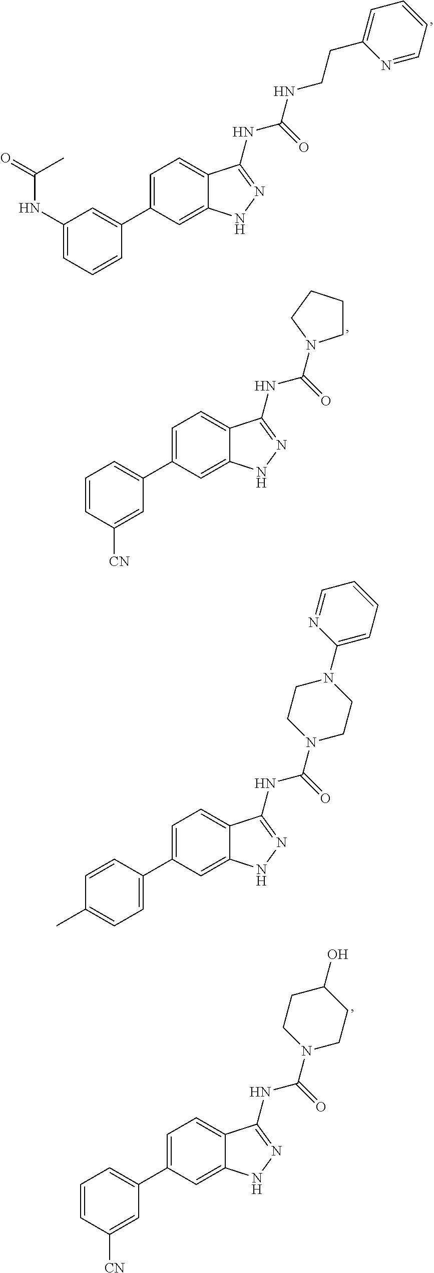 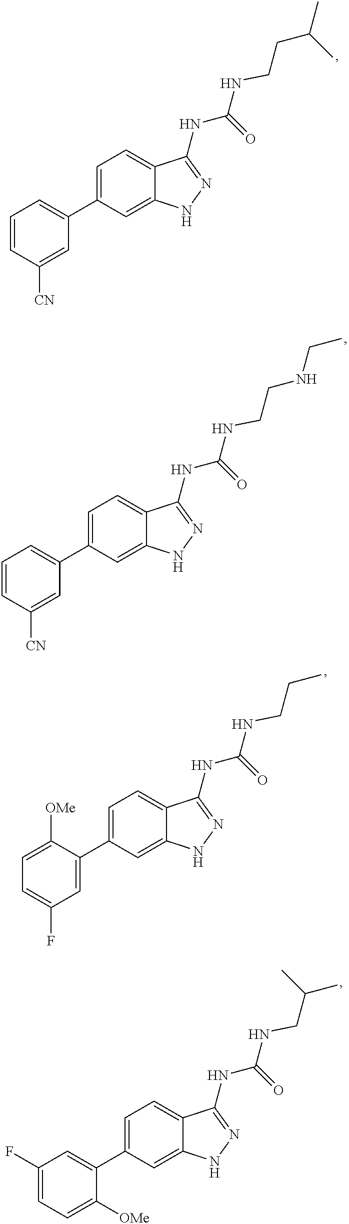 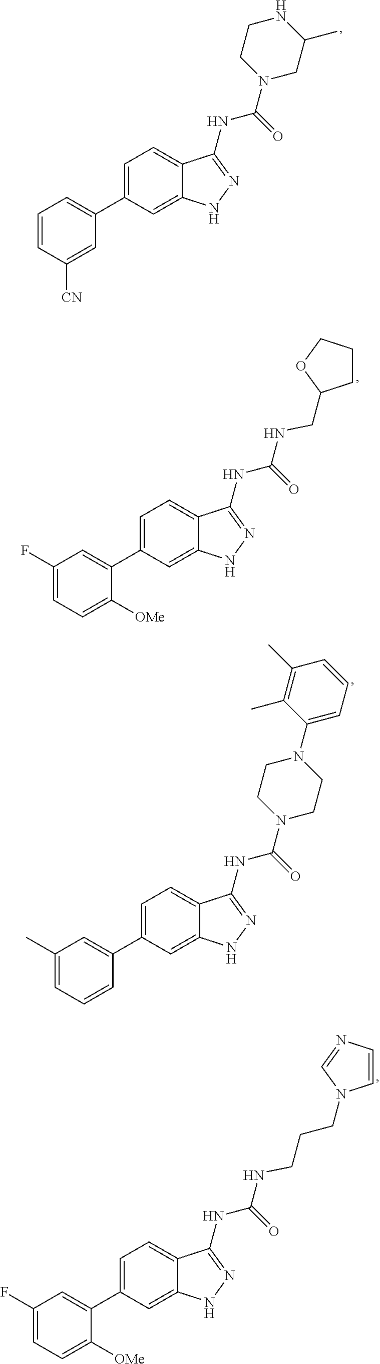 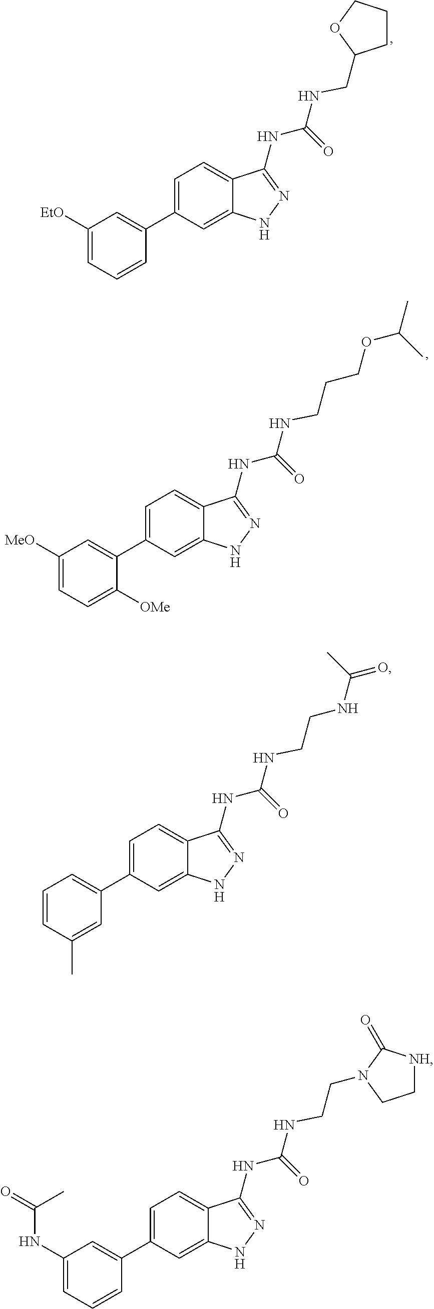 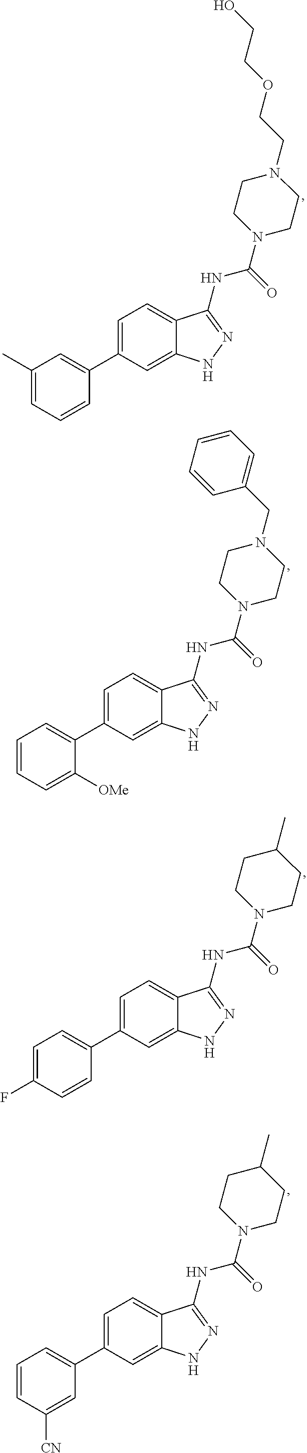 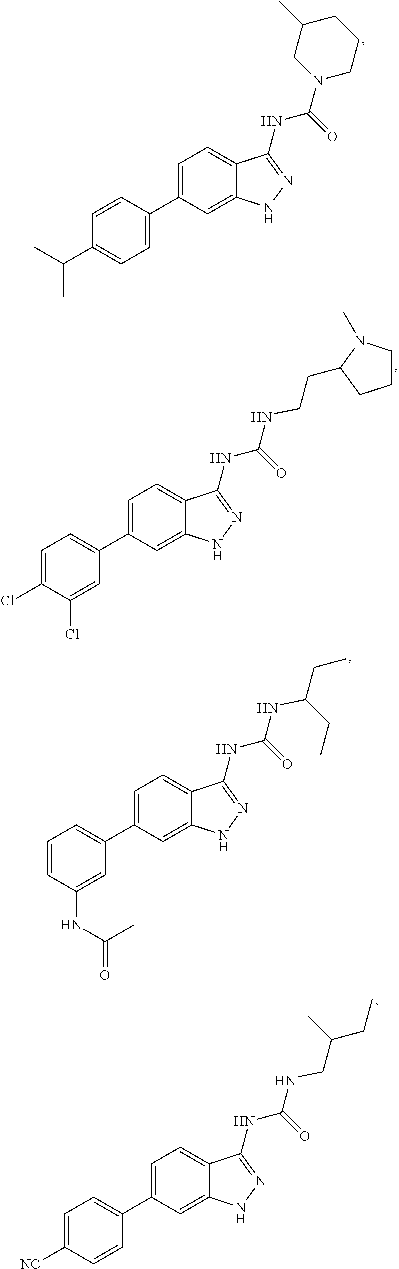 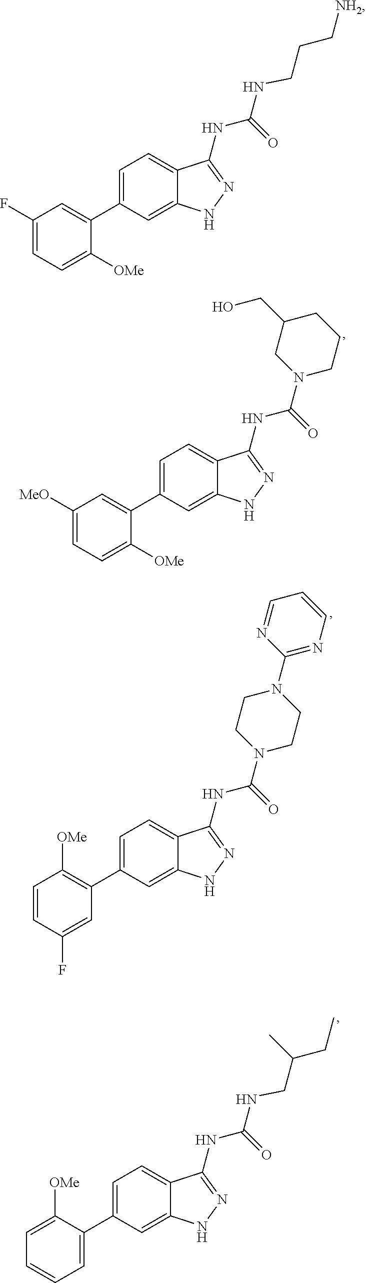 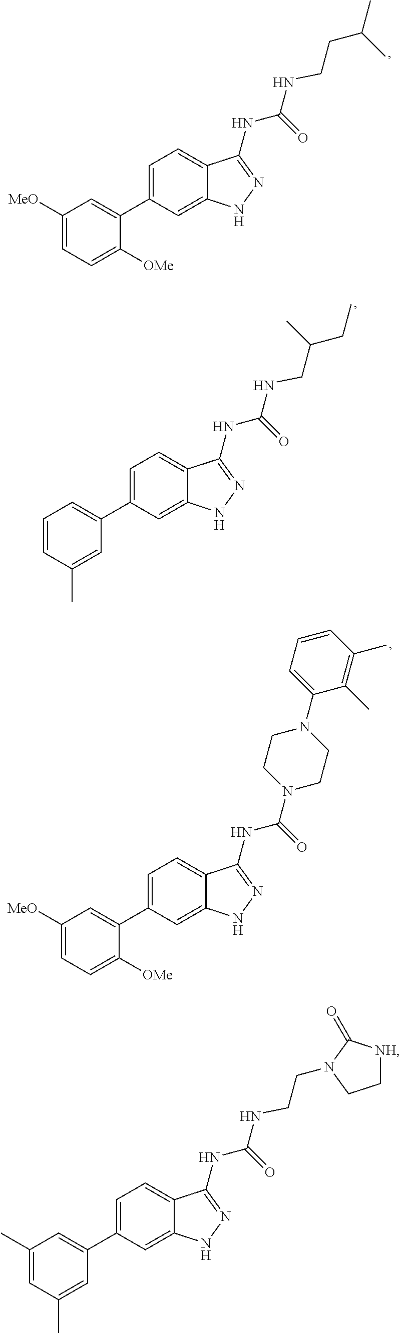 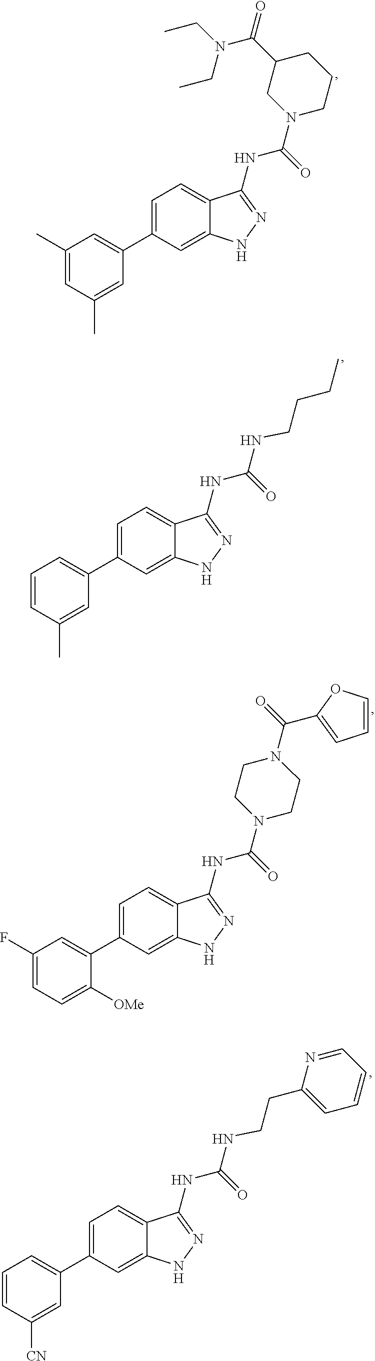 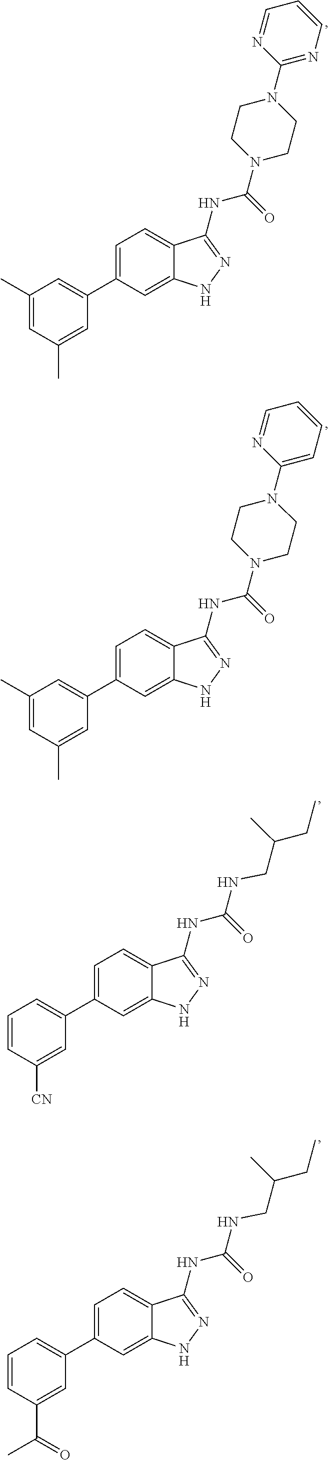 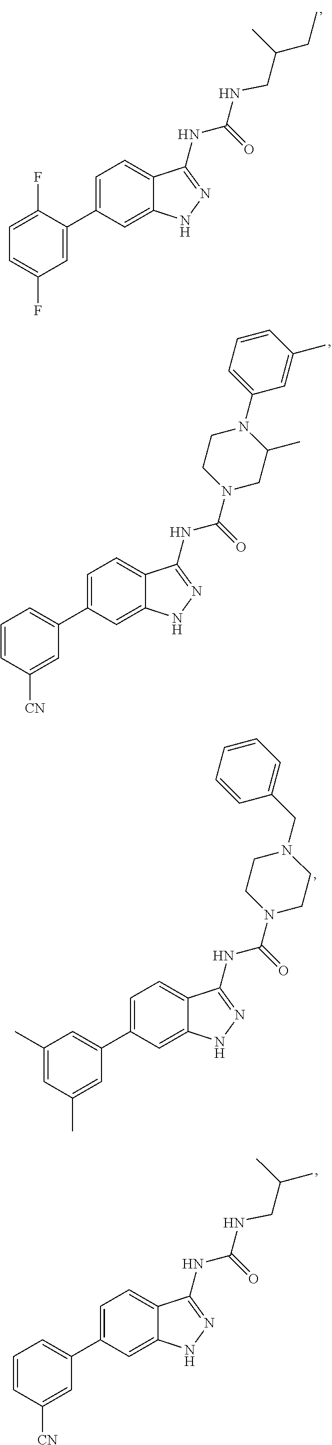 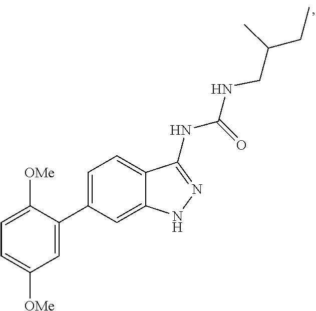 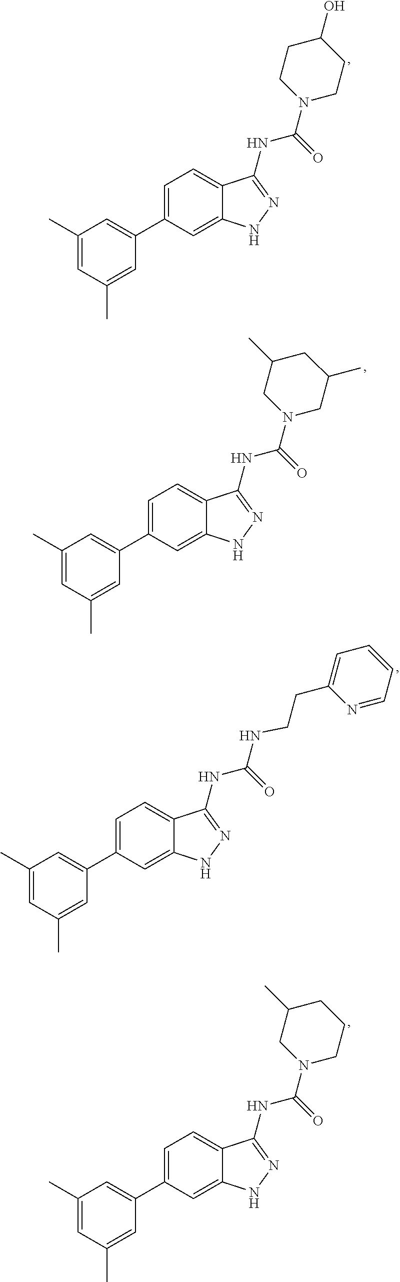 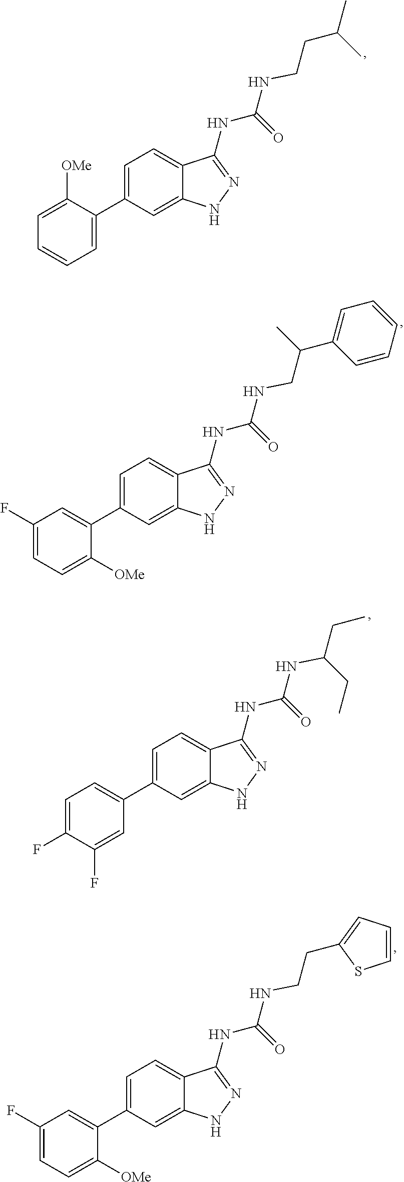 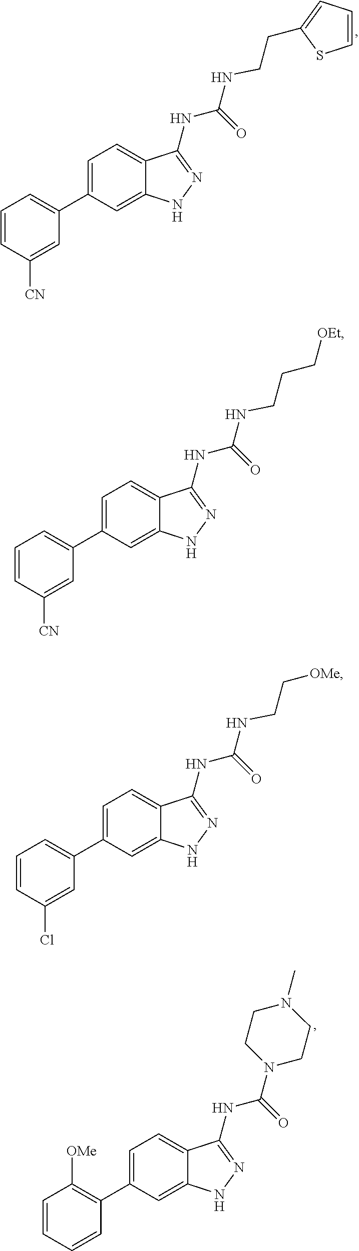 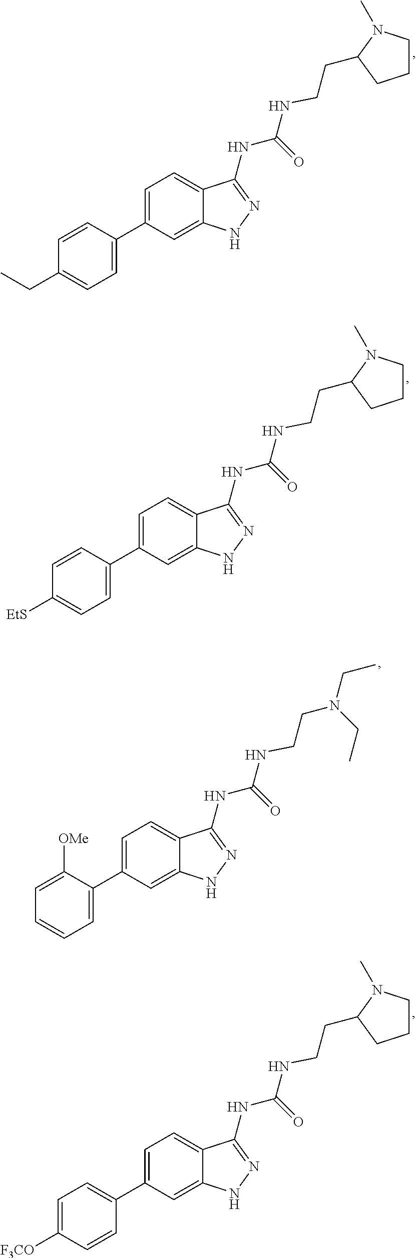 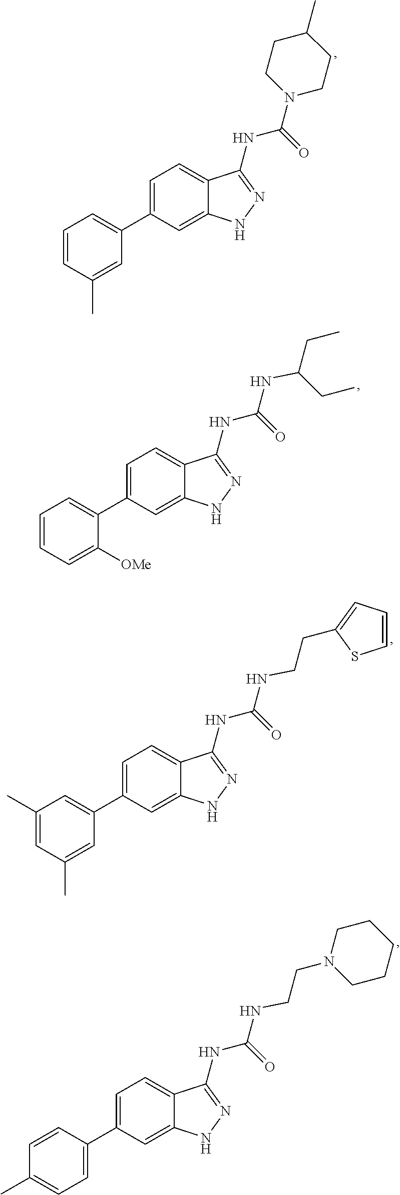 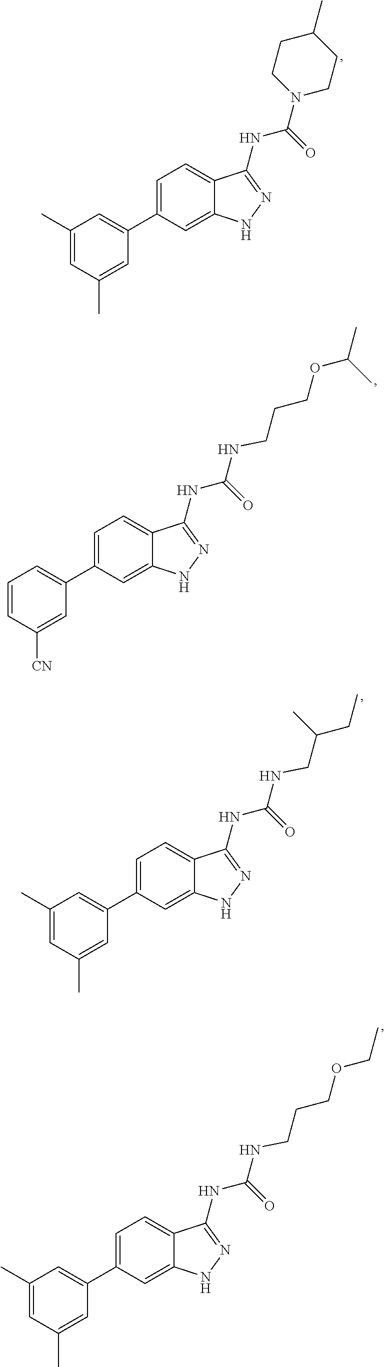 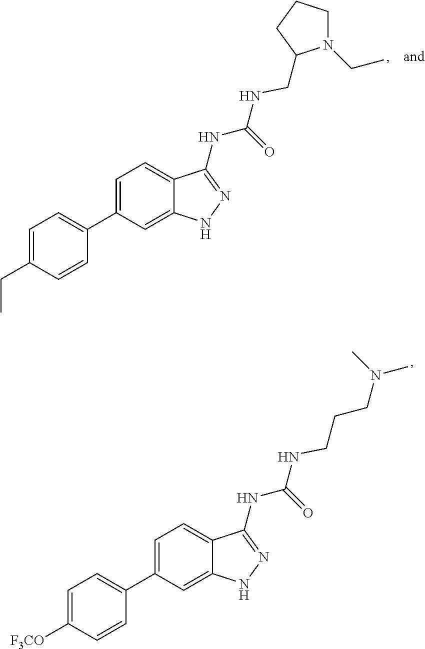
or a pharmaceutically acceptable salt or prodrug thereof.

47. A method of treating a disorder or disease in which aberrant Wnt signaling is implicated in a patient, the method comprising administering to the patient a therapeutically effective amount of a compound having a structure of Formula IV, or a pharmaceutically acceptable salt or pro-drug thereof: 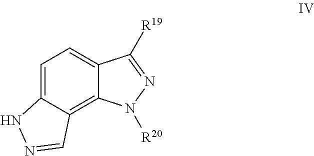
wherein each R⁹ is independently selected from the group consisting of H, —C₁₋₉ alkyl, —CF₃, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;
each R¹⁰ is independently selected from the group consisting of —C₁₋₉ alkyl, —CF₃, —(C₁₋₉ alkyl)ₙcarbocyclylR¹², —(C₁₋₉ alkyl)ₙheterocyclylR¹², —(C₁₋₉ alkyl)ₙarylR¹² and —(C₁₋₉ alkyl)ₙheteroarylR¹²;
each R¹¹ is independently selected from the group consisting of CN, —OR⁹ and R⁹;
each R¹² is 1-5 substituents each selected from the group consisting of H, C₁₋₉alkyl, -alkylaminoalkyl, halide, —CF₃, carbocyclylR¹², heterocyclylR¹², arylR¹², heteroarylR¹², —(C₁₋₉ alkyl)ₙOR⁹, —(C₁₋₉alkyl)ₙSR⁹, —(C₁₋₉alkyl)ₙS(═O)R¹⁰, —(C₁₋₉ alkyl)ₙSO₂R⁹, —(C₁₋₉ alkyl)ₙN(R⁹)SO₂R⁹, —(C₁₋₉ alkyl)ₙSO₂N(R⁹)₂, —(C₁₋₉ alkyl)ₙN(R⁹)₂, —(C₁₋₉ alkyl)ₙN(R⁹)C(=A)N(R⁹)₂, —(C₁₋₉ alkyl)ₙC(=A)N(R⁹)₂, —(C₁₋₉ alkyl)ₙNR⁹C(═O)OR⁹, —(C₁₋₉ alkyl)ₙN(R⁹)C(=A)R⁹, —(C₁₋₉ alkyl)ₙOC(═O)N(R⁹)₂, —NO₂, —CN, —(C₁₋₉ alkyl)ₙCO₂R⁹ and —(C₁₋₉ alkyl)ₙC(=A)R⁹;
R¹⁹ is independently selected from the group consisting of C₁₋₉alkyl, —OR⁹, amino, —S(═O)R¹⁰, —SO₂R⁹, —N(R⁹)S(═O)R¹⁰, —N(R⁹)SO₂R⁹, —SO₂N(R⁹)₂, —N(R⁹)₂, —N(R⁹)C(═O)N(R⁹)₂, —NR⁹C(═O)OR⁹, —C(═O)N(R⁹)₂, —N(R⁹)C(═O)R⁹, —OC(═O)N(R⁹)₂, —CO₂R⁹ and —C(═O)R⁹;
R²⁰ is independently selected from the group consisting of arylR¹² and heteroarylR¹²;
each A is independently selected from O, S and NR¹¹; and
each n is 0 or 1.

48. The method of claim 47 wherein n is 0.

49. The method of claim 47 wherein n is 1.

50. The method of claim 47 wherein A is O.

51. The method of claim 47 wherein R¹⁹ is —C(═O)N(R⁹)₂.

52. The method of claim 47 wherein R¹⁹ is —C(═O)NH₂.

53. The method of claim 47 wherein R¹⁹ is —C(═O)NHR⁹.

54. The method of claim 47 wherein R²⁰ is arylR¹².

55. The method of claim 47 wherein R²⁰ is heterocyclylR¹².

56. The method of claim 47, wherein the compound has a structure selected from the group consisting of: 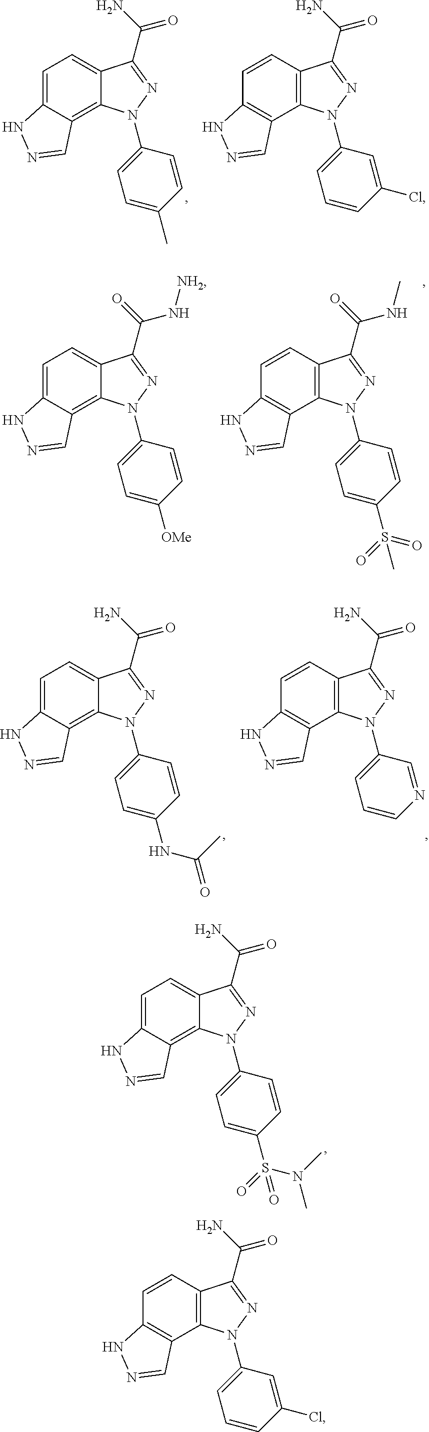 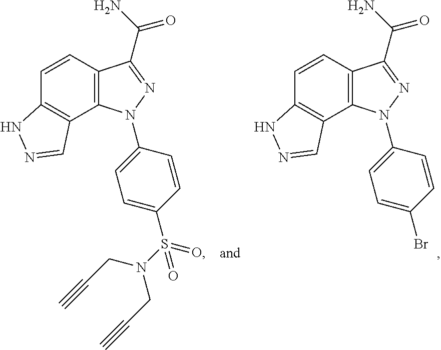
or a pharmaceutically acceptable salt or prodrug thereof.

57. A pharmaceutical composition comprising a therapeutically effective amount of a compound according to any of the formulas I, II, III, or IV, as set forth herein, or a pharmaceutically acceptable salt thereof, and a pharmaceutically acceptable excipient:

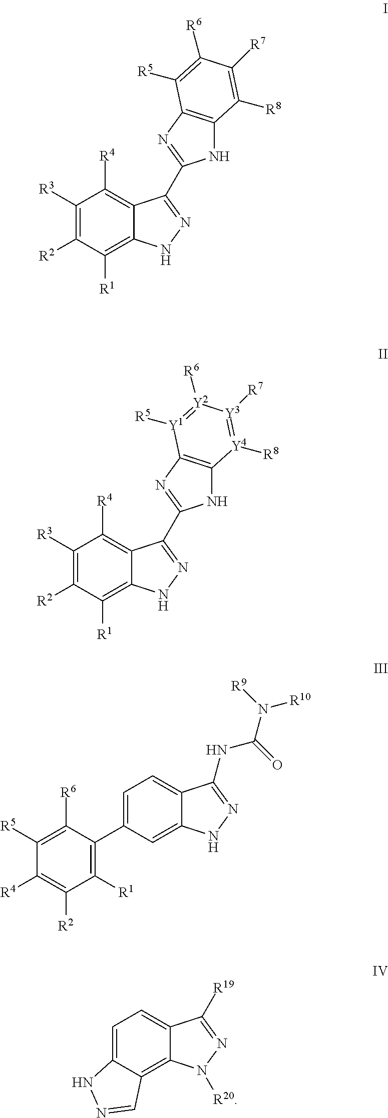

58. A method according to any of the claim 1, 17, 31 or 47, wherein the disorder or disease is cancer.

59. A method according to any of the claim 1, 17, 31 or 47, wherein the disorder or disease is diabetic retinopathy.

60. A method according to any of the claim 1, 17, 31 or 47, wherein the disorder or disease is neovascular glaucoma.

61. A method according to any of the claim 1, 17, 31 or 47, wherein the disorder or disease is rheumatoid arthritis.

62. A method according to any of the claim 1, 17, 31 or 47, wherein the disorder or disease is psoriasis.

63. A method according to any of the claim 1, 17, 31 or 47, wherein the disorder or disease is a mycotic or viral infection.

64. A method according to any of the claim 1, 17, 31 or 47, wherein the disorder or disease is osteochondrodysplasia.

65. A method according to any of the claim 1, 17, 31 or 47, wherein the disorder or disease is Alzheimer's disease.

66. A method according to any of the claim 1, 17, 31 or 47, wherein the disorder or disease is osteoarthritis.

67. A method according to any of the claim 1, 17, 31 or 47, wherein the disorder or disease is a genetic disease caused by mutations in Wnt signaling components, wherein the genetic disease is chosen from: polyposis coli, osteoporosis-pseudoglioma syndrome, familial exudative vitreoretinopathy, retinal angiogenesis, early coronary disease, tetra-amelia syndrome, Müllerian-duct regression and virilization, SERKAL syndrome, diabetes mellitus type 2, Fuhrmann syndrome, Al-Awadi/Raas-Rothschild/Schinzel phocomelia syndrome, odonto-onycho-dermal dysplasia, obesity, split-hand/foot malformation, caudal duplication syndrome, tooth agenesis, Wilms tumor, skeletal dysplasia, focal dermal hypoplasia, autosomal recessive anonychia, neural tube defects, alpha-thalassemia (ATRX) syndrome, fragile X syndrome, ICF syndrome, Angelman syndrome, Prader-Willi syndrome, Beckwith-Wiedemann Syndrome and Rett syndrome.

68. The method according to any of the claim 1, 17, 31 or 47, wherein the patient is a human.

69. The method of claim 58, wherein the cancer is chosen from: hepatocellular carcinoma, colon cancer, breast cancer, pancreatic cancer leukemia, lymphoma, sarcoma and ovarian cancer.

70. The method according to any of the claim 1, 17, 31 or 47, wherein the compound inhibits one or more proteins in the Wnt pathway.

71. The method according to any of the claim 1, 17, 31 or 47, wherein the compound inhibits signaling induced by one or more Wnt proteins.

72. The method of claim 71, wherein the Wnt proteins are chosen from: WNT1, WNT2, WNT2B, WNT3, WNT3A, WNT4. WNT5A, WNT5B, WNT6, WNT7A, WNT7B, WNT8A, WNT8B, WNT9A, WNT9B, WNT10A, WNT10B, WNT11, and WNT16.

73. The method according to any of the claim 1, 17, 31 or 47, wherein the compound inhibits a kinase activity.

74. The method of claim 73, wherein the kinase activity mediates a disease or disorder comprising tumor growth, cell proliferation, or angiogenesis.

75. The method of claim 74, wherein the protein kinase is from the CDK, VEGF, CLK, HIPK, Abl, JAK or CHK families of kinases.

76. A method according to any of the claim 1, 17, 31 or 47, wherein the disorder or disease is associated with aberrant cellular proliferation.

77. A method according to any of the claim 1, 17, 31 or 47, wherein the method prevents or reduces angiogenesis in a patient.

78. A method according to any of the claim 1, 17, 31 or 47, wherein the method prevents or reduces abnormal cellular proliferation in a patient.
